(19) United States
(12) Patent Application Publication
(10) Pub. No.: US 2013/0345079 A1
(43) Pub. Date: Dec. 26, 2013

(54) MYCOBACTERIUM TUBERCULOSIS ANTIGENS AND COMBINATIONS THEREOF HAVING HIGH SEROREACTIVITY

(71) Applicants: Gregory C. Ireton, Seattle, WA (US); Steven G. Reed, Bellevue, WA (US)

(72) Inventors: Gregory C. Ireton, Seattle, WA (US); Steven G. Reed, Bellevue, WA (US)

(73) Assignee: INFECTIOUS DISEASE RESEARCH INSTITUTE, Seattle, WA (US)

(21) Appl. No.: 13/880,025

(22) PCT Filed: Aug. 16, 2011
(86) PCT No.: PCT/US2011/047859

§ 371 Date: Sep. 13, 2013

Related U.S. Application Data
(60) Provisional application No. 61/407,308, filed on Oct. 27, 2010.

(51) Int. Cl. G01N 33/569

(52) U.S. Cl. CPC G01N 33/5695
USPC 506/9; 435/7.1; 530/350; 536/23.4; 435/7.92; 506/18

(57) ABSTRACT

The present invention relates to compositions and fusion proteins containing comprising Mycobacterium sp. antigens, and polynucleotides encoding such compositions and fusion proteins. The invention also relates to methods for their use in the diagnosis, treatment and/or prevention of tuberculosis infection.

### CROSS-REFERENCE TO RELATED APPLICATION

The present application claims the benefit under 35 U.S.C. §119(e) of U.S. Provisional Patent Application No. 61/407,308 filed on Oct. 27, 2010.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480239_{—}428PC_SEQUENCE_LISTING.txt. The text file is 229 KB, was created on Aug. 16, 2011, and is being submitted electronically via EFS-Web, concurrent with the filing of the specification.

### BACKGROUND OF THE INVENTION

1. Technical Field

The present invention relates generally to compositions comprising antigenic and/or immunogenic combinations of Mycobacterium tuberculosis antigens and their use in the diagnosis, treatment and/or prevention of tuberculosis.

2. Description of the Related Art

Tuberculosis (TB) is a chronic infectious disease caused by Mycobacterium tuberculosis (Mtb) and is one of the leading causes of mortality due to infectious disease worldwide (Arch Intern Med 163:1009-21, 2003). Nearly one-third of the world's population is believed to be infected, with approximately 8.8 million new cases detected each year (Journal of Infectious Diseases 196 Suppl 1:S15-27, 2007). The World Health Organization (WHO) cites TB as the single most important fatal infection, with over 1.6 million deaths per year, the majority (95%) in developing countries (Global Tuberculosis Control: Surveilance, Planning, Financing., Vol. 376. World Health Organization, 2007).

Because of logistical and technical shortcomings, human TB testing in most countries is limited to clinical evaluation of symptomatic individual and screening high-risk populations. Compounding the severity of TB is the realization that a leading cause of death among HIV-positive people is concomitant TB, accounting for about one-third of AIDS-related deaths. It is estimated that a rapid and widely available diagnostic with 85% sensitivity and 95% specificity would result in 400,000 fewer deaths each year and would greatly reduce the global health cost of TB (Nature 444 Suppl 1:49-57, 2006).

The existing TB diagnostic methods are either time-consuming, or complex and labor-intensive, or inaccurate, or too expensive for routine use in resource limited settings (Am J Respir Crit. Care Med 162:1323-9, 2000; Arch Pathol Lab Med 123:1101-3, 1999). For active pulmonary disease, sputum smear microscopy, culture, and/or PCR-based probes can be used to support X-ray findings and/or clinical observations suggestive of TB. Of these, microscopic examination of sputum is the only rapid, relatively simple, and inexpensive test for TB. The reported sensitivity of the Ziehl-Neelsen staining of unprocessed sputum smears in immunocompetent adults is only 40-70% (Am Rev Respir Dis 129:264-8, 1984; Chest 95:1193-7, 1989), and it may be significantly lower in children and/or HIV-infected patients (Tuber Lung Dis 74:191-4, 1993). The delayed or missed TB diagnosis certainly contributes to Mtb transmission and increased mortality (Int J Tuberc Lung Dis 5:233-9, 2001; Clin Infect Dis 21:1170-4, 1995).

Mycobacterial culture is the gold standard method of TB diagnosis. However, it requires up to 8 weeks for the isolation of Mtb from a clinical specimen and, importantly, in 10-20% of positive cases the bacillus is not successfully cultured (Lancet 356:1099-104, 2000). Culture is more expensive than microscopy and requires a high standard of technical expertise. Therefore, a sensitive and specific point-of-care test for the rapid diagnosis of patients with active TB would facilitate early treatment and reduce Mtb transmission.

An antibody test for TB has long been sought. Serologic assays remain attractive for use in resource-limited settings, because they generally are simple, rapid, and relatively inexpensive, compared to other methods. In TB, serological tests may also offer the possibility of detecting cases that are usually missed by routine sputum smear microscopy, such as extra-pulmonary disease and pediatric TB. Numerous serological assays for TB have been developed over the years using a variety of antigens to detect circulating antibodies, including complement fixation tests, haemagglutination tests, radioimmunoassay, and enzyme-linked immunosorbent assays (ELISAs) (Health Technol Assess 11:1-196, 2007; PLoS Med 4:e202, 2007; Thorax 62:911-8, 2007; Future Microbiol 2:355-9, 2007). Both lateral flow and enzyme immunoassay formats have been developed and are currently available commercially, but none so far has demonstrated adequate sensitivity and specificity (Tuber Lung Dis 80:131-40, 2000; J Clin Microbiol 40:1989-93, 2002; J Clin Microbiol 38:2227-31, 2000; PLoS Med 4:e202, 2007).

Accordingly, there remains a need for improved reagents and methods for effectively and reproducibly diagnosing, preventing and/or treating tuberculosis. The present invention fulfills these needs and offers other related advantages.

### BRIEF SUMMARY OF THE INVENTION

The present invention relates generally to compositions comprising combinations of seroreactive antigens, fusion polypeptides comprising the antigens and polynucleotides encoding the antigens and fusion polypeptides, where the antigens are from a Mycobacterium species, particularly Mycobacterium tuberculosis. The present invention also relates methods of using the polypeptides and polynucleotides of the invention, particularly in the serological-based diagnosis of Mycobacterium infection. For example, the antigens of the invention, when employed in combination and/or as fusion polypeptides or polynucleotides as described herein, represent improved diagnostic markers for tuberculosis based on the seroreactive patterns identified for the antigens.

For example, in one aspect of the invention, there are provided diagnostic compositions comprising a combination of Mycobacterium tuberculosis seroreactive antigens (e.g., a combination of two or more, or three or more, seroreactive antigens), or immunogenic fragments thereof, wherein the antigens are selected from the group consisting of Rv0054 (SEQ ID NO: 1), Rv0164 (SEQ ID NO: 3), Rv0410 (SEQ ID NO: 5), Rv0455c (SEQ ID NO: 7), Rv0632 (SEQ ID NO: 9) Rv0655 (SEQ ID NO: 11), Rv0831c (SEQ ID NO: 13), Rv0934 (SEQ ID NO: 15), Rv0952 (SEQ ID NO: 17), Rv1009 (SEQ ID NO: 19), Rv1099 (SEQ ID NO: 21), Rv1240 (SEQ ID NO: 23), Rv1288 (SEQ ID NO: 25), Rv1410c (SEQ ID NO: 27), ), Rv1411 (SEQ ID NO: 29) Rv1569 (SEQ ID NO: 31), Rv1789 (SEQ ID NO: 33), Rv1813c (SEQ ID NO: 35), Rv1827 (SEQ ID NO: 37), Rv1837 (SEQ ID NO: 39), Rv1860 (SEQ ID NO: 41), Rv1886c (SEQ ID NO: 43), Rv1908 (SEQ ID NO: 45), Rv1980 (SEQ ID NO:47), Rv1984c (SEQ ID NO: 49), Rv2031 (SEQ ID NO: 51), Rv2032 (SEQ ID NO: 53), Rv2220 (SEQ ID NO: 55), Rv2450 (SEQ ID NO: 57), Rv2608 (SEQ ID NO: 59), Rv2623 (SEQ ID NO: 61), Rv2866 (SEQ ID NO: 63), Rv2873 (SEQ ID NO: 65), Rv2875 (SEQ ID NO: 67), Rv3020 (SEQ ID NO: 69), Rv3044 (SEQ ID NO: 71), Rv3310 (SEQ ID NO: 73), Rv3407 (SEQ ID NO: 75), Rv3611 (SEQ ID NO: 77), Rv3614 (SEQ ID NO: 79), Rv3616 (SEQ ID NO: 81) Rv3619 (SEQ ID NO: 83), Rv3628 (SEQ ID NO: 85), Rv3804 (SEQ ID NO:87), Rv3841 (SEQ ID NO: 89), Rv3864 (SEQ ID NO:91), Rv3874 (SEQ ID NO: 93) and Rv3881 (SEQ ID NO: 95), and antigens having at least 90% identity to any of the foregoing sequences.

In a more specific embodiment, the seroreactive antigens are selected from the group consisting of Rv0455 (SEQ ID NO: 7), Rv0632 (SEQ ID NO:9), Rv0831 (SEQ ID NO: 13), Rv0934 (SEQ ID NO: 15), ), Rv1240 (SEQ ID NO: 23), Rv1410 (SEQ ID NO: 27), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3310 (SEQ ID NO: 73), ), Rv3619 (SEQ ID NO: 83), Rv3864 (SEQ ID NO:91), Rv3874 (SEQ ID NO: 93), Rv3881 (SEQ ID NO: 95), and antigens having at least 90% identity to any of the foregoing sequences.

In another more specific embodiment, the seroreactive antigens are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences.

The combination of antigens described herein can include a combination of separate recombinant antigens, or immunogenic fragments thereof. Alternatively, the combination of antigens, or immunogenic fragments thereof, may be covalently linked in the form of a fusion polypeptide.

Therefore, according to another aspect of the invention, there are also provided isolated fusion polypeptides comprising a combination of, for example, two or more, or three or more, covalently linked Mycobacterium tuberculosis antigens, or immunogenic fragments thereof, wherein the antigens are selected from the group consisting of Rv0054 (SEQ ID NO: 1), Rv0164 (SEQ ID NO: 3), Rv0410 (SEQ ID NO: 5), Rv0455c (SEQ ID NO: 7), Rv0632 (SEQ ID NO: 9) Rv0655 (SEQ ID NO: 11), Rv0831c (SEQ ID NO: 13), Rv0934 (SEQ ID NO: 15), Rv0952 (SEQ ID NO: 17), Rv1009 (SEQ ID NO: 19), Rv1099 (SEQ ID NO: 21), Rv1240 (SEQ ID NO: 23), Rv1288 (SEQ ID NO: 25), Rv1410c (SEQ ID NO: 27), ), Rv1411 (SEQ ID NO: 29) Rv1569 (SEQ ID NO: 31), Rv1789 (SEQ ID NO: 33), Rv1813c (SEQ ID NO: 35), Rv1827 (SEQ ID NO: 37), Rv1837 (SEQ ID NO: 39), Rv1860 (SEQ ID NO: 41), Rv1886c (SEQ ID NO: 43), Rv1908 (SEQ ID NO: 45), Rv1980 (SEQ ID NO:47), Rv1984c (SEQ ID NO: 49), Rv2031 (SEQ ID NO: 51), Rv2032 (SEQ ID NO: 53), Rv2220 (SEQ ID NO: 55), Rv2450 (SEQ ID NO: 57), Rv2608 (SEQ ID NO: 59), Rv2623 (SEQ ID NO: 61), Rv2866 (SEQ ID NO: 63), Rv2873 (SEQ ID NO: 65), Rv2875 (SEQ ID NO: 67), Rv3020 (SEQ ID NO: 69), Rv3044 (SEQ ID NO: 71), Rv3310 (SEQ ID NO: 73), Rv3407 (SEQ ID NO: 75), Rv3611 (SEQ ID NO: 77), Rv3614 (SEQ ID NO: 79), Rv3616 (SEQ ID NO: 81) Rv3619 (SEQ ID NO: 83), Rv3628 (SEQ ID NO: 85), Rv3804 (SEQ ID NO:87), Rv3841 (SEQ ID NO: 89), Rv3864 (SEQ ID NO:91), Rv3874 (SEQ ID NO: 93) and Rv3881 (SEQ ID NO: 95), and antigens having at least 90% identity to any of the foregoing sequences.

In certain more specific embodiments, the fusion polypeptide comprises a combination of three or more covalently linked Mycobacterium tuberculosis seroreactive antigens, or immunogenic fragments thereof, wherein the antigens are selected from the group consisting of Rv0455 (SEQ ID NO: 7), Rv0632 (SEQ ID NO:9), Rv0831 (SEQ ID NO: 13), Rv0934 (SEQ ID NO: 15), ), Rv1240 (SEQ ID NO: 23), Rv1410 (SEQ ID NO: 27), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3310 (SEQ ID NO: 73), ), Rv3619 (SEQ ID NO: 83), Rv3864 (SEQ ID NO:91), Rv3874 (SEQ ID NO: 93), Rv3881 (SEQ ID NO: 95), and antigens having at least 90% identity to any of the foregoing sequences.

In other more specific embodiments, the fusion polypeptide comprises a combination of three or more covalently linked Mycobacterium tuberculosis seroreactive antigens, or immunogenic fragments thereof, wherein the antigens are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences.

Certain specific fusion polypeptides of the invention comprise seroreactive sequences from Rv2031 (SEQ ID NO: 51), Rv0934 (SEQ ID NO: 15) and Rv2032 (SEQ ID NO: 53). One such preferred fusion polypeptide is referred to as DID90A, having a sequence set forth in SEQ ID NO: 97. Other specific fusion polypeptides of the invention comprise seroreactive sequences from Rv2875 (SEQ ID NO: 67), Rv0934 (SEQ ID NO: 15) and Rv2032 (SEQ ID NO: 53), such as the fusion polypeptide referred to as DID90B, having a sequence set forth in SEQ ID NO: 98. Still other specific fusion polypeptides of the invention comprise seroreactive sequences from Rv0831 (SEQ ID NO: 13), Rv0934 (SEQ ID NO: 15) and Rv2032 (SEQ ID NO: 53), such as the fusion polypeptide referred to as DID104, having a sequence set forth in SEQ ID NO: 99. Still other specific fusion polypeptides of the invention comprise seroreactive sequences from Rv2031 (SEQ ID NO: 51), Rv0934 (SEQ ID NO: 15) and Rv3874 (SEQ ID NO: 93), such as the fusion polypeptide referred to as DID64, having a sequence set forth in SEQ ID NO: 100. Still other specific fusion polypeptides of the invention comprise seroreactive sequences from Rv2875 (SEQ ID NO: 67), Rv0934 (SEQ ID NO: 15) and Rv3874 (SEQ ID NO: 93), such as the fusion polypeptide referred to as DID65, having a sequence set forth in SEQ ID NO: 101. Still other specific fusion polypeptides of the invention comprise seroreactive sequences from Rv2875 (SEQ ID NO: 67), Rv1860 (SEQ ID NO: 41) and Rv2032 (SEQ ID NO: 53), such as the fusion polypeptide referred to as DID82, having a sequence set forth in SEQ ID NO: 102. Still other specific fusion polypeptides of the invention comprise seroreactive sequences from Rv0632 (SEQ ID NO: 9), Rv1980 (SEQ ID NO: 47) and Rv3881 (SEQ ID NO: 95), such as the fusion polypeptide referred to as DID96, having a sequence set forth in SEQ ID NO:103. Still other specific fusion polypeptides of the invention comprise seroreactive sequences from Rv1860 (SEQ ID NO: 41), Rv1980 (SEQ ID NO: 47) and Rv3864 (SEQ ID NO: 91), such as the fusion polypeptide referred to as DID94, having a sequence set forth in SEQ ID NO: 104.

In many diagnostic embodiments of the invention, the seroreactive antigens of the invention, whether present as separate antigens or covalently linked in the form of one or more fusion polypeptides, are preferably immobilized on a solid support. For example, in certain preferred embodiments, the seroreactive antigens are immobilized on a solid support in an assay format selected from an ELISA assay, a lateral flow test strip assay, a dual path platform assay, or other rapid diagnostic test format. In other preferred embodiments, the seroreactive antigens are used in a test-of-cure method, kit or composition, as described herein, for monitoring the status of infection in an infected individual over time and/or in response to treatment.

The present invention also provides, according to another aspect, isolated polynucleotides encoding the antigen combinations and/or fusion polypeptides described herein.

According to yet another aspect of the present invention, there are provide methods for detecting Mycobacterium tuberculosis in a biological sample, comprising (a) contacting the biological sample with a combination of Mycobacterium tuberculosis seroreactive antigens (e.g., a combination of two or more, or three or more, seroreactive antigens), or immunogenic fragments thereof, or fusion polypeptides thereof, wherein the antigens are selected from the group consisting of Rv0054 (SEQ ID NO: 1), Rv0164 (SEQ ID NO: 3), Rv0410 (SEQ ID NO: 5), Rv0455c (SEQ ID NO: 7), Rv0632 (SEQ ID NO: 9) Rv0655 (SEQ ID NO: 11), Rv0831c (SEQ ID NO: 13), Rv0934 (SEQ ID NO: 15), Rv0952 (SEQ ID NO: 17), Rv1009 (SEQ ID NO: 19), Rv1099 (SEQ ID NO: 21), Rv1240 (SEQ ID NO: 23), Rv1288 (SEQ ID NO: 25), Rv1410c (SEQ ID NO: 27), ), Rv1411 (SEQ ID NO: 29) Rv1569 (SEQ ID NO: 31), Rv1789 (SEQ ID NO: 33), Rv1813c (SEQ ID NO: 35), Rv1827 (SEQ ID NO: 37), Rv1837 (SEQ ID NO: 39), Rv1860 (SEQ ID NO: 41), Rv1886c (SEQ ID NO: 43), Rv1908 (SEQ ID NO: 45), Rv1980 (SEQ ID NO:47), Rv1984c (SEQ ID NO: 49), Rv2031 (SEQ ID NO: 51), Rv2032 (SEQ ID NO: 53), Rv2220 (SEQ ID NO: 55), Rv2450 (SEQ ID NO: 57), Rv2608 (SEQ ID NO: 59), Rv2623 (SEQ ID NO: 61), Rv2866 (SEQ ID NO: 63), Rv2873 (SEQ ID NO: 65), Rv2875 (SEQ ID NO: 67), Rv3020 (SEQ ID NO: 69), Rv3044 (SEQ ID NO: 71), Rv3310 (SEQ ID NO: 73), Rv3407 (SEQ ID NO: 75), Rv3611 (SEQ ID NO: 77), Rv3614 (SEQ ID NO: 79), Rv3616 (SEQ ID NO: 81) Rv3619 (SEQ ID NO: 83), Rv3628 (SEQ ID NO: 85), Rv3804 (SEQ ID NO:87), Rv3841 (SEQ ID NO: 89), Rv3864 (SEQ ID NO:91), Rv3874 (SEQ ID NO: 93) and Rv3881 (SEQ ID NO: 95), and antigens having at least 90% identity to any of the foregoing sequences; and (b) detecting in the biological sample the presence of antibodies that bind thereto.

In certain embodiments of the diagnostic methods of the invention, the seroreactive antigens are selected from the group consisting of Rv0455 (SEQ ID NO: 7), Rv0632 (SEQ ID NO:9), Rv0831 (SEQ ID NO: 13), Rv0934 (SEQ ID NO: 15), ), Rv1240 (SEQ ID NO: 23), Rv1410 (SEQ ID NO: 27), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3310 (SEQ ID NO: 73), ), Rv3619 (SEQ ID NO: 83), Rv3864 (SEQ ID NO:91), Rv3874 (SEQ ID NO: 93), Rv3881 (SEQ ID NO: 95), and antigens having at least 90% identity to any of the foregoing sequences.

In certain other embodiments of the diagnostic methods of the invention, the seroreactive antigens are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences.

Certain specific fusion polypeptides for use in the methods of the invention comprise seroreactive sequences from Rv2031 (SEQ ID NO: 51), Rv0934 (SEQ ID NO: 15) and Rv2032 (SEQ ID NO: 53), such as the fusion polypeptide referred to as DID90A, having a sequence set forth in SEQ ID NO: 97. Other specific fusion polypeptides for use in the methods of the invention comprise seroreactive sequences from Rv2875 (SEQ ID NO: 67), Rv0934 (SEQ ID NO: 15) and Rv2032 (SEQ ID NO: 53), such as the fusion polypeptide referred to as DID90B, having a sequence set forth in SEQ ID NO: 98. Still other specific fusion polypeptides for use in the methods of the invention comprise seroreactive sequences from Rv0831 (SEQ ID NO: 13), Rv0934 (SEQ ID NO: 15) and Rv2032 (SEQ ID NO: 53), such as the fusion polypeptide referred to as DID104, having a sequence set forth in SEQ ID NO: 99. Still other specific fusion polypeptides for use in the methods of the invention comprise seroreactive sequences from Rv2031 (SEQ ID NO: 51), Rv0934 (SEQ ID NO: 15) and Rv3874 (SEQ ID NO: 93), such as the fusion polypeptide referred to as DID64, having a sequence set forth in SEQ ID NO: 100. Still other specific fusion polypeptides of the invention comprise seroreactive sequences from Rv2875 (SEQ ID NO: 67), Rv0934 (SEQ ID NO: 15) and Rv3874 (SEQ ID NO: 93), such as the fusion polypeptide referred to as DID65, having a sequence set forth in SEQ ID NO: 101. Still other specific fusion polypeptides of the invention comprise seroreactive sequences from Rv2875 (SEQ ID NO: 67), Rv1860 (SEQ ID NO: 41) and Rv2032 (SEQ ID NO: 53), such as the fusion polypeptide referred to as DID82, having a sequence set forth in SEQ ID NO: 102. Still other specific fusion polypeptides of the invention comprise seroreactive sequences from Rv0632 (SEQ ID NO: 9), Rv1980 (SEQ ID NO: 47) and Rv3881 (SEQ ID NO: 95), such as the fusion polypeptide referred to as DID96, having a sequence set forth in SEQ ID NO: 103. Still other specific fusion polypeptides of the invention comprise seroreactive sequences from Rv1860 (SEQ ID NO: 41), Rv1980 (SEQ ID NO: 47) and Rv3864 (SEQ ID NO: 91), such as the fusion polypeptide referred to as DID94, having a sequence set forth in SEQ ID NO: 104

In certain preferred embodiments, the methods and/or kits of the invention take the form of a rapid diagnostic test, such as a lateral flow test strip device or dual path platform device, wherein the seroreactive antigens, or fusions thereof, are immobilized on a solid support. Therefore, according to another aspect, the present invention provides a lateral flow diagnostic test strip for detecting Mycobacterium tuberculosis infection in a biological sample, comprising a combination of Mycobacterium tuberculosis seroreactive antigens, or immunogenic portions or fusions thereof, as described herein, immobilized on a solid support material. In certain more specific embodiments, the seroreactive antigens immobilized on the test strip in a lateral flow or dual path platform assay are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences. In other more specific embodiments, at least some of the seroreactive antigens immobilized on the test strip are covalently linked in the form of a fusion polypeptide, such as a fusion polypeptide selected from the group consisting of DID90A (SEQ ID NO: 97), DID90B (SEQ ID NO: 98), DID104 (SEQ ID NO: 99), DID64 (SEQ ID NO: 100), DID65 (SEQ ID NO: 101), DID82 (SEQ ID NO: 102), DID96 (SEQ ID NO: 103), and DID94 (SEQ ID NO: 104), or a sequence having at least 90% identity thereto.

### BRIEF DESCRIPTION OF THE DRAWINGS

FIG. 1A shows SDS-PAGE analysis of purified recombinant M. tuberculosis proteins. Individual Mtb proteins are listed by their H37Rv gene number. 2 to 5 ug of each antigen was run on a 4-20% SDS-PAGE and stained by Coomassie to determine relative purity. M=molecular weight size standards (160, 120, 80, 60, 40, 25, 20, 15, 10 kDa).

FIG. 1B shows serum reactivity in recombinant M. tuberculosis protein arrays. 79 Mtb proteins were printed in duplicate, incubated with TB+ or NEC sera, developed and scanned. Representative images of 3 control sera (A) and 3 TB+ sera (B) from protein arrays are shown.

FIG. 2 shows ELISA responses to recombinant Mtb proteins. TB+, confirmed sputum positive pulmonary TB samples (N=92) from Brazil; NEC=negative, non-endemic (US) control sera (N=46). Representative data for 24 recombinant Mtb antigens is shown. The median OD is represented by the crossing line of within the samples. Individual antigens are listed below, with positive reactivity determined as those samples giving ELISA optical density readings 2-fold above the mean of the negative controls and greater than an OD₄₅₀ₙₘ=0.2.

FIG. 3 shows fusion protein Serum ELISA results. Results for DID90A, DID90B, DID104, TBF10 and the individual component antigens displayed as a box plot. TB+=confirmed pulmonary TB samples (N=36) from India; NEC=negative, non-endemic (US) control sera (N=30); EC=negative, Indian endemic control sera (N=20). Each box represents 20 data from the 25th to the 75th percentile, the median is represented by the crossing line and the whiskers extend to the lowest and highest values.

FIG. 4 shows Mtb antigen reactivity in the MAPIA. The 4 fusion polyproteins and 6 single antigens were printed on nitrocellulose membranes (listed on the right) and the assay was performed as described in Methods. Each strip represents one serum sample and displays antigen reactivity pattern. Results are shown for 10 negative control sera (on the left) and 30 sera from TB patients including 6 from India and 24 from Brazil (on the right).

### BRIEF DESCRIPTION OF SEQUENCE IDENTIFIERS

SEQ ID NO: 1 represents an amino acid sequence of the Mtb antigen referred to as Rv0054.

SEQ ID NO: 2 represents a polynucleotide sequence encoding the polypeptide of SEQ ID NO: 1.

SEQ ID NO: 3 represents an amino acid sequence of the Mtb antigen referred to as Rv0164.

SEQ ID NO: 4 represents a polynucleotide sequence encoding the polypeptide of SEQ ID NO: 3.

SEQ ID NO: 5 represents an amino acid sequence of the Mtb antigen referred to as Rv0410.

SEQ ID NO: 6 represents a polynucleotide sequence encoding the polypeptide of SEQ ID NO: 5.

SEQ ID NO: 7 represents an amino acid sequence of the Mtb antigen referred to as Rv0455c.

SEQ ID NO: 8 represents a polynucleotide sequence encoding the polypeptide of SEQ ID NO: 7.

SEQ ID NO: 9 represents an amino acid sequence of the Mtb antigen referred to as Rv0632.

SEQ ID NO: 10 represents a polynucleotide sequence encoding the polypeptide of SEQ ID NO: 9.

SEQ ID NO: 11 represents an amino acid sequence of the Mtb antigen referred to as Rv0655.

SEQ ID NO: 12 represents a polynucleotide sequence encoding the polypeptide of SEQ ID NO: 11.

SEQ ID NO: 13 represents an amino acid sequence of the Mtb antigen referred to as Rv0831 c.

SEQ ID NO: 14 represents a polynucleotide sequence encoding the polypeptide of SEQ ID NO: 13.

SEQ ID NO: 15 represents an amino acid sequence of the Mtb antigen referred to as Rv0934.

SEQ ID NO: 16 represents a polynucleotide sequence encoding the polypeptide of SEQ ID NO: 15.

SEQ ID NO: 17 represents an amino acid sequence of the Mtb antigen referred to as Rv0952.

SEQ ID NO: 18 represents a polynucleotide sequence encoding the polypeptide of SEQ ID NO: 17.

SEQ ID NO: 19 represents an amino acid sequence of the Mtb antigen referred to as Rv1009.

SEQ ID NO: 20 represents a polynucleotide sequence encoding the polypeptide of SEQ ID NO: 19.

SEQ ID NO: 21 represents an amino acid sequence of the Mtb antigen referred to as Rv1099.

SEQ ID NO: 22 represents a polynucleotide sequence encoding the polypeptide of SEQ ID NO: 21.

SEQ ID NO: 23 represents an amino acid sequence of the Mtb antigen referred to as Rv1240.

SEQ ID NO: 24 represents a polynucleotide sequence encoding the polypeptide of SEQ ID NO: 23.

SEQ ID NO: 25 represents an amino acid sequence of the Mtb antigen referred to as Rv1288.

SEQ ID NO: 26 represents a polynucleotide sequence encoding the polypeptide of SEQ ID NO: 25.

SEQ ID NO: 27 represents an amino acid sequence of the Mtb antigen referred to as Rv1410c.

SEQ ID NO: 28 represents a polynucleotide sequence encoding the polypeptide of SEQ ID NO: 27.

SEQ ID NO: 29 represents an amino acid sequence of the Mtb antigen referred to as Rv1411.

SEQ ID NO: 30 represents a polynucleotide sequence encoding the polypeptide of SEQ ID NO: 29.

SEQ ID NO: 31 represents an amino acid sequence of the Mtb antigen referred to as Rv1569.

SEQ ID NO: 32 represents a polynucleotide sequence encoding the polypeptide of SEQ ID NO: 31.

SEQ ID NO: 33 represents an amino acid sequence of the Mtb antigen referred to as Rv1789.

SEQ ID NO: 34 represents a polynucleotide sequence encoding the polypeptide of SEQ ID NO: 33.

SEQ ID NO: 35 represents an amino acid sequence of the Mtb antigen referred to as Rv1813c.

SEQ ID NO: 36 represents a polynucleotide sequence encoding the polypeptide of SEQ ID NO: 35.

SEQ ID NO: 37 represents an amino acid sequence of the Mtb antigen referred to as Rv1827.

SEQ ID NO: 38 represents a polynucleotide sequence encoding the polypeptide of SEQ ID NO: 37.

SEQ ID NO: 39 represents an amino acid sequence of the Mtb antigen referred to as Rv1837.

SEQ ID NO: 40 represents a polynucleotide sequence encoding the polypeptide of SEQ ID NO: 39.

SEQ ID NO: 41 represents an amino acid sequence of the Mtb antigen referred to as Rv1860.

SEQ ID NO: 42 represents a polynucleotide sequence encoding the polypeptide of SEQ ID NO: 41.

SEQ ID NO: 43 represents an amino acid sequence of the Mtb antigen referred to as Rv1886c.

SEQ ID NO: 44 represents a polynucleotide sequence encoding the polypeptide of SEQ ID NO: 43.

SEQ ID NO: 45 represents an amino acid sequence of the Mtb antigen referred to as Rv1908.

SEQ ID NO: 46 represents a polynucleotide sequence encoding the polypeptide of SEQ ID NO: 45.

SEQ ID NO: 47 represents an amino acid sequence of the Mtb antigen referred to as Rv1980.

SEQ ID NO: 48 represents a polynucleotide sequence encoding the polypeptide of SEQ ID NO: 47.

SEQ ID NO: 49 represents an amino acid sequence of the Mtb antigen referred to as Rv1984c.

SEQ ID NO: 50 represents a polynucleotide sequence encoding the polypeptide of SEQ ID NO: 49.

SEQ ID NO: 51 represents an amino acid sequence of the Mtb antigen referred to as Rv2031.

SEQ ID NO: 52 represents a polynucleotide sequence encoding the polypeptide of SEQ ID NO: 51.

SEQ ID NO: 53 represents an amino acid sequence of the Mtb antigen referred to as Rv2032.

SEQ ID NO: 54 represents a polynucleotide sequence encoding the polypeptide of SEQ ID NO: 53.

SEQ ID NO: 55 represents an amino acid sequence of the Mtb antigen referred to as Rv2220.

SEQ ID NO: 56 represents a polynucleotide sequence encoding the polypeptide of SEQ ID NO: 55.

SEQ ID NO: 57 represents an amino acid sequence of the Mtb antigen referred to as Rv2450.

SEQ ID NO: 58 represents a polynucleotide sequence encoding the polypeptide of SEQ ID NO: 57.

SEQ ID NO: 59 represents an amino acid sequence of the Mtb antigen referred to as Rv2608.

SEQ ID NO: 60 represents a polynucleotide sequence encoding the polypeptide of SEQ ID NO: 59.

SEQ ID NO: 61 represents an amino acid sequence of the Mtb antigen referred to as Rv2623.

SEQ ID NO: 62 represents a polynucleotide sequence encoding the polypeptide of SEQ ID NO: 61.

SEQ ID NO: 63 represents an amino acid sequence of the Mtb antigen referred to as Rv2866.

SEQ ID NO: 64 represents a polynucleotide sequence encoding the polypeptide of SEQ ID NO: 63.

SEQ ID NO: 65 represents an amino acid sequence of the Mtb antigen referred to as Rv2873.

SEQ ID NO: 66 represents a polynucleotide sequence encoding the polypeptide of SEQ ID NO: 65.

SEQ ID NO: 67 represents an amino acid sequence of the Mtb antigen referred to as Rv2875.

SEQ ID NO: 68 represents a polynucleotide sequence encoding the polypeptide of SEQ ID NO: 67.

SEQ ID NO: 69 represents an amino acid sequence of the Mtb antigen referred to as Rv3020.

SEQ ID NO: 70 represents a polynucleotide sequence encoding the polypeptide of SEQ ID NO: 69.

SEQ ID NO: 71 represents an amino acid sequence of the Mtb antigen referred to as Rv3044.

SEQ ID NO: 72 represents a polynucleotide sequence encoding the polypeptide of SEQ ID NO: 71.

SEQ ID NO: 73 represents an amino acid sequence of the Mtb antigen referred to as Rv3310.

SEQ ID NO: 74 represents a polynucleotide sequence encoding the polypeptide of SEQ ID NO: 73.

SEQ ID NO: 75 represents an amino acid sequence of the Mtb antigen referred to as Rv3407.

SEQ ID NO: 76 represents a polynucleotide sequence encoding the polypeptide of SEQ ID NO: 75.

SEQ ID NO: 77 represents an amino acid sequence of the Mtb antigen referred to as Rv3611.

SEQ ID NO: 78 represents a polynucleotide sequence encoding the polypeptide of SEQ ID NO: 77.

SEQ ID NO: 79 represents an amino acid sequence of the Mtb antigen referred to as Rv3614.

SEQ ID NO: 80 represents a polynucleotide sequence encoding the polypeptide of SEQ ID NO: 79.

SEQ ID NO: 81 represents an amino acid sequence of the Mtb antigen referred to as Rv3616.

SEQ ID NO: 82 represents a polynucleotide sequence encoding the polypeptide of SEQ ID NO: 81.

SEQ ID NO: 83 represents an amino acid sequence of the Mtb antigen referred to as Rv3619.

SEQ ID NO: 84 represents a polynucleotide sequence encoding the polypeptide of SEQ ID NO: 83.

SEQ ID NO: 85 represents an amino acid sequence of the Mtb antigen referred to as Rv3628.

SEQ ID NO: 86 represents a polynucleotide sequence encoding the polypeptide of SEQ ID NO: 85.

SEQ ID NO: 87 represents an amino acid sequence of the Mtb antigen referred to as Rv3804.

SEQ ID NO: 88 represents a polynucleotide sequence encoding the polypeptide of SEQ ID NO: 87.

SEQ ID NO: 89 represents an amino acid sequence of the Mtb antigen referred to as Rv3841.

SEQ ID NO: 90 represents a polynucleotide sequence encoding the polypeptide of SEQ ID NO: 89.

SEQ ID NO: 91 represents an amino acid sequence of the Mtb antigen referred to as Rv3864.

SEQ ID NO: 92 represents a polynucleotide sequence encoding the polypeptide of SEQ ID NO: 91.

SEQ ID NO: 93 represents an amino acid sequence of the Mtb antigen referred to as Rv3874.

SEQ ID NO: 94 represents a polynucleotide sequence encoding the polypeptide of SEQ ID NO: 93.

SEQ ID NO: 95 represents an amino acid sequence of the Mtb antigen referred to as Rv3881.

SEQ ID NO: 96 represents a polynucleotide sequence encoding the polypeptide of SEQ ID NO: 95.

SEQ ID NO: 97 represents an amino acid sequence of the fusion polypeptide referred to as DID90A, which contains sequences from Rv2031, Rv0934 and Rv2032.

SEQ ID NO: 98 represents an amino acid sequence of the fusion polypeptide referred to as DID90B, which contains sequences from Rv2875, Rv0934 and Rv2032.

SEQ ID NO: 99 represents an amino acid sequence of the fusion polypeptide referred to as DID104, which contains sequences from Rv0831, Rv0934 and Rv2032.

SEQ ID NO: 100 represents an amino acid sequence of the fusion polypeptide referred to as DID64, which contains sequences from Rv2031, Rv0934 and Rv3874.

SEQ ID NO: 101 represents an amino acid sequence of the fusion polypeptide referred to as DID65, which contains sequences from Rv2875, Rv0934, and Rv3874.

SEQ ID NO: 102 represents an amino acid sequence of the fusion polypeptide referred to as DID82, which contains sequences from Rv2875, Rv1860, and Rv2032.

SEQ ID NO: 103 represents an amino acid sequence of the fusion polypeptide referred to as DID96, which contains sequences from Rv0632, Rv1980, and Rv3881.

SEQ ID NO: 104 represents an amino acid sequence of the fusion polypeptide referred to as DID94, which contains sequences from Rv1860, Rv1980, and Rv3864.

### DETAILED DESCRIPTION OF THE INVENTION

The present invention relates generally to highly seroreactive compositions comprising Mycobacterium antigens. The compositions of the present invention generally comprise a combination of heterologous polypeptides of a Mycobacterium species of the tuberculosis complex. A Mycobacterium species of the tuberculosis complex includes those species traditionally considered as causing the disease tuberculosis, as well as Mycobacterium environmental and opportunistic species that cause tuberculosis and lung disease in immune compromised patients, such as patients with AIDS, e.g., Mycobacterium tuberculosis (Mtb), Mycobacterium bovis, or Mycobacterium africanum, BCG, Mycobacterium avium, Mycobacterium intracellulare, Mycobacterium celaturn, Mycobacterium genavense, Mycobacterium haemophilum, Mycobacterium kansasii, Mycobacterium simiae, Mycobacterium vaccae, Mycobacterium fortuitum, and Mycobacterium scrofulaceum (see, e.g., Harrison's Principles of Internal Medicine, volume 1, pp. 1004-1014 and 1019-1020). In a preferred embodiment, the Mycobacterium species to be diagnosed, prevented or treated according to the invention is Mycobacterium tuberculosis (Mtb). The sequences of proteins from Mycobacterium species are readily available. For example, Mycobacterium tuberculosis sequences can be found in Cole et al., Nature 393:537 (1998) and can be found at websites such as those maintained by the Wellcome Trust Sanger Institute and Institut Pasteur.

### A. Seroreactive Mycobacterium Antigens and Fusions Thereof

The present invention, in one aspect, provides combinations of isolated Mycobacterium polypeptides, as described herein, as well as fusion polypeptides comprising such antigens and compositions containing the same. As described herein, the polypeptides of the invention have been demonstrated to be highly reactive with antibodies from the sera of patients infected with Mycobacterium tuberculosis. Moreover, the present invention has defined various subsets of seroreactive antigens which, when used in combination, or as fusion polypeptides, provide improved sensitivity and specificity in the detection of tuberculosis infection in a patient. As described herein, the seroreactive antigen combinations, fusions, compositions, methods and kits of the invention are particularly advantageous when used in the context of rapid point-of-care diagnostic testing formats, such as lateral flow, dual path platform and ELISA formats.

Generally, a polypeptide of the invention will be an isolated polypeptide and may be a fragment (e.g., an antigenic/immunogenic portion) from an amino acid sequence disclosed herein, or may comprise an entire amino acid sequence disclosed herein. Polypeptides of the invention, antigenic/immunogenic fragments thereof, and other variants may be prepared using conventional recombinant and/or synthetic techniques.

In certain embodiments, the polypeptides of the invention are immunogenic, i.e., they react detectably within an immunoassay (such as an ELISA or T cell stimulation assay) with sera, antisera and/or T cells from an infected subject. In certain preferred embodiments, the polypeptides of the invention react detectably within an immunoassay with sera from an infected subject, i.e., they are seroreactive. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, sera, antisera and/or T cell lines or clones. As used herein, sera, antisera and antibodies are “antigen-specific” if they specifically bind to an antigen (i.e., they react with the protein in an immunoassay, and do not react detectably with unrelated proteins). Such sera, antisera and antibodies may be prepared as described herein, and using well-known techniques.

In a particular embodiment, an immunogenic portion of a polypeptide of the present invention is a portion that reacts with sera, antisera and/or T cells at a level that is not substantially less than the reactivity of the full-length polypeptide (e.g., in an ELISA and/or T cell reactivity assay). Preferably, the level of immunogenic activity (e.g., seroreactivity) of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

A polypeptide composition of the invention may also comprise one or more polypeptides that are immunologically reactive with antibodies and/or T-cells generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting antibodies and/or T-cells that are immunologically reactive with one or more polypeptides described herein, or one or more polypeptides encoded by contiguous polynucleotide sequences contained in the polynucleotide sequences disclosed herein, or immunogenic fragments or variants thereof, or to one or more polynucleotide sequences which hybridize to one or more of these sequences under conditions of moderate to high stringency.

The present invention also provides, in other embodiments, polypeptide fragments, including immunogenic fragments (e.g., seroreactive fragments), comprising at least about 5, 10, 15, 20, 25, 50, or 100 contiguous amino acids, or more, including all intermediate lengths, of a polypeptide composition set forth herein, or those encoded by a polynucleotide sequence set forth herein.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequence set forth herein. In certain preferred embodiments, the variants retain the same or substantially the same level of seroreactivity as observed for a wild-type or other reference polypeptide.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., about 1-30 amino acids) has been removed from the N- and/or C-terminal of a mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Nat'l Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Nat'l Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

In certain embodiments of the invention, there are provided Mycobacterium tuberculosis fusion polypeptides comprising a selected combination of seroreactive antigens, as described herein, linked together in the form of a single molecule. More specifically, a fusion polypeptide will typically contain at least two, at least three, at least four, or at least five, or more, heterologous Mycobacterium sp. seroreactive sequences, such as the Mycobacterium tuberculosis seroreactive antigen sequences described herein, covalently linked, either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

Fusion polypeptides or fusion proteins can also include conservatively modified variants, polymorphic variants, alleles, mutants, subsequences, interspecies homologs, and immunogenic fragments of the antigens that make up the fusion protein. Mycobacterium tuberculosis antigens are described in Cole et al., Nature 393:537 (1998), which discloses the entire Mycobacterium tuberculosis genome. Antigens from other Mycobacterium species that correspond to Mycobacterium tuberculosis antigens can be identified, e.g., using sequence comparison algorithms, as described herein, or other methods known to those of skill in the art, e.g., hybridization assays and antibody binding assays.

The fusion polypeptides of the invention, in addition to comprising sequences derived from the seroreactive antigens described herein, may further comprise other unrelated sequences or chemical moieties, such as a sequence or moiety that assists in, e.g., immobilizing the polypeptide on a solid support, providing T helper epitopes (an immunological fusion partner) and/or that assists in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques. Preferably, a fusion protein is expressed as a recombinant protein. For example, DNA sequences encoding the polypeptide components of a desired fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Within certain embodiments, an immunological fusion partner for use in a fusion polypeptide of the invention is derived from protein D, a surface protein of the gram-negative bacterium Haemophilus influenza B (WO 91/18926). For example, a protein D derivative comprises approximately the first third of the protein (e.g., the first N-terminal 100 110 amino acids), and a protein D derivative may be lipidated. Within certain embodiments, the first 109 residues of a lipoprotein D fusion partner is included on the N-terminus to provide the polypeptide with additional exogenous T cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, an immunological fusion partner comprises an amino acid sequence derived from the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292 (1986)). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798 (1992)). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### B. Polynucleotide Compositions

The present invention also provides isolated polynucleotides, particularly those encoding the seroreactive antigens and fusion polypeptides of the invention, as well as compositions comprising such polynucleotides. As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a Mycobacterium antigen or a portion thereof) or may comprise a variant, or a biological or antigenic functional equivalent of such a sequence. Polynucleotide variants may contain, for example, one or more substitutions, additions, deletions and/or insertions, as further described below, preferably such that the immunogenicity of the encoded polypeptide is not diminished, relative to the native protein. The effect on the immunogenicity of the encoded polypeptide may generally be assessed as described herein. The term “variants” also encompasses homologous genes of xenogenic origin.

In additional embodiments, the present invention provides isolated polynucleotides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200 500; 500 1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, with the total length preferably being limited by the ease of preparation and use in the intended recombinant DNA protocol.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Mycobacterium polynucleotides and fusions thereof may be prepared, manipulated and/or expressed using any of a variety of well established techniques known and available in the art.

For example, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, expression and/or immunogenicity of the gene product.

In order to express a desired polypeptide, a nucleotide sequence encoding the polypeptide, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of—galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk− or aprt− cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol. 55:121-131 (1995)).

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins.

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

### C. Diagnostic Methods and Kits

As noted above, in certain preferred aspects of the invention, the compositions, fusion polypeptides and/or polynucleotides described herein may be used as diagnostic reagents for detecting and/or monitoring Mycobacterium tuberculosis infection in a patient. For example, the compositions, fusion polypeptides and polynucleotides of the invention may be used in any of a number of in vitro and in vivo assays for detecting or evaluating the presence of Mycobacterium tuberculosis for diagnosis of infection, monitoring of disease progression, test-of-cure evaluation, and the like.

As demonstrated herein, the present invention has identified various preferred combinations of seroreactive antigens that offer improved sensitivity and specificity in serological tests for detecting tuberculosis infection. Therefore, in certain embodiments, the invention provides compositions for diagnosing Mycobacterium tuberculosis infection using, for example, a serological-based assay, such as a rapid lateral flow diagnostic assay or a dual path platform diagnostic assay. Generally, the diagnostic compositions will comprise a plurality of seroreactive antigens, i.e., at least two, at least three, at least four, at least five or at least six, or more, seroreactive antigens, such as those selected from the group consisting of Rv0054 (SEQ ID NO: 1), Rv0164 (SEQ ID NO: 3), Rv0410 (SEQ ID NO: 5), Rv0455c (SEQ ID NO: 7), Rv0632 (SEQ ID NO: 9) Rv0655 (SEQ ID NO: 11), Rv0831c (SEQ ID NO: 13), Rv0934 (SEQ ID NO: 15), Rv0952 (SEQ ID NO: 17), Rv1009 (SEQ ID NO: 19), Rv1099 (SEQ ID NO: 21), Rv1240 (SEQ ID NO: 23), Rv1288 (SEQ ID NO: 25), Rv1410c (SEQ ID NO: 27), ), Rv1411 (SEQ ID NO: 29) Rv1569 (SEQ ID NO: 31), Rv1789 (SEQ ID NO: 33), Rv1813c (SEQ ID NO: 35), Rv1827 (SEQ ID NO: 37), Rv1837 (SEQ ID NO: 39), Rv1860 (SEQ ID NO: 41), Rv1886c (SEQ ID NO: 43), Rv1908 (SEQ ID NO: 45), Rv1980 (SEQ ID NO:47), Rv1984c (SEQ ID NO: 49), Rv2031 (SEQ ID NO: 51), Rv2032 (SEQ ID NO: 53), Rv2220 (SEQ ID NO: 55), Rv2450 (SEQ ID NO: 57), Rv2608 (SEQ ID NO: 59), Rv2623 (SEQ ID NO: 61), Rv2866 (SEQ ID NO: 63), Rv2873 (SEQ ID NO: 65), Rv2875 (SEQ ID NO: 67), Rv3020 (SEQ ID NO: 69), Rv3044 (SEQ ID NO: 71), Rv3310 (SEQ ID NO: 73), Rv3407 (SEQ ID NO: 75), Rv3611 (SEQ ID NO: 77), Rv3614 (SEQ ID NO: 79), Rv3616 (SEQ ID NO: 81) Rv3619 (SEQ ID NO: 83), Rv3628 (SEQ ID NO: 85), Rv3804 (SEQ ID NO:87), Rv3841 (SEQ ID NO: 89), Rv3864 (SEQ ID NO:91), Rv3874 (SEQ ID NO: 93) and Rv3881 (SEQ ID NO: 95), and sequences having at least 90% identity to any of the foregoing sequences. Of course, it will be understood that a seroreactive antigen may also comprise an immunogenic fragment or variant of a seroreactive antigen, as described herein.

In more specific embodiments, the seroreactive antigens, or immunogenic fragments or variants thereof, used in a method of the present invention are selected from the group consisting of Rv0455 (SEQ ID NO: 7), Rv0632 (SEQ ID NO:9), Rv0831 (SEQ ID NO: 13), Rv0934 (SEQ ID NO: 15), ), Rv1240 (SEQ ID NO: 23), Rv1410 (SEQ ID NO: 27), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3310 (SEQ ID NO: 73), ), Rv3619 (SEQ ID NO: 83), Rv3864 (SEQ ID NO:91), Rv3874 (SEQ ID NO: 93), Rv3881 (SEQ ID NO: 95), and antigens having at least 90% identity to any of the foregoing sequences.

In other more specific embodiments, the seroreactive antigens are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences.

The seroreactive antigens, or immunogenic fragments or variants thereof, may be used in essentially any diagnostic kit or assay format desired, e.g., as individual antigens assayed separately, as multiple antigens assays simultaneously, as antigens immobilized on a solid support such as an array or membrane, or the like.

In still other embodiments of the invention, there are provided diagnostic kits for detecting Mycobacterium tuberculosis infection in a biological sample, comprising (a) a polypeptide comprising at least an immunogenic portion of an antigen or fusion polypeptide described herein, and (b) a detection reagent. In a preferred embodiment, the kit comprises at least two, at least three, at least four, at least five, or at least six, or more, seroreactive antigens or immunogenic fragments or variants or fusions thereof, as described herein.

In another embodiment, there are provided diagnostic kits for detecting Mycobacterium tuberculosis infection in a biological sample, comprising (a) an antibody or antigen binding fragment thereof that is specific for a polypeptide comprising at least an immunogenic portion of an antigen or fusion polypeptide described herein, and (b) a detection reagent.

In other embodiments, methods are provided for detecting the presence of Mycobacterium tuberculosis infection in a biological sample, comprising (a) contacting a biological sample with an antibody that binds to an antigen or fusion polypeptide described herein; and (b) detecting in the biological sample the presence of Mycobacterium tuberculosis proteins that bind to the monoclonal antibody.

In still other embodiments, methods are provided for detecting Mycobacterium tuberculosis infection in a biological sample, comprising (a) contacting the biological sample with an antigen combination or fusion polypeptide as described herein and (b) detecting in the biological sample the presence of antibodies that bind to the antigens or fusion polypeptide. In a preferred embodiment, the biological sample is patient blood or sera.

There are a variety of assay formats known to those of ordinary skill in the art for using purified antigens or fusion polypeptides to detect antibodies in a sample. See, e.g., Harlow and Lane, Antibodies: A Laboratory Manual, Cold Spring Harbor Laboratory, 1988. In one embodiment, the assay involves the use of polypeptide immobilized on a solid support to bind to and remove the antibody from the sample. The bound antibody may then be detected using a detection reagent that binds to the antibody/peptide complex and contains a detectable reporter group. Suitable detection reagents include, for example, antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be essentially any solid material known to those of ordinary skill in the art to which the seroreactive antigens or fusion polypeptides may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The antigens or fusion polypeptides may be bound to the solid support using any of a variety of techniques known and available in the art. The term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred in some embodiments. In such cases, adsorption may be achieved by contacting the polypeptide, in a suitable buffer, with the solid support for a suitable amount of time.

In certain embodiments, the diagnostic assay employed is an enzyme linked immunosorbent assay (ELISA). This assay may be performed by first contacting antigens that have been immobilized on a solid support, commonly the well of a microtiter plate, with the sample, such as patient sera, such that antibodies to the polypeptide within the sample are allowed to bind to the immobilized polypeptide. Unbound sample may then be removed from the immobilized polypeptide and a detection reagent capable of binding to the immobilized antibody-polypeptide complex may be added. The amount of detection reagent that remains bound to the solid support is then determined using a method appropriate for the specific detection reagent.

Once the polypeptide is immobilized on the support, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized polypeptide is then incubated with the sample, and antibody (if present in the sample) is allowed to bind to the antigen. The sample may be diluted with a suitable diluent, such as phosphate-buffered saline (PBS) prior to incubation. In general, an appropriate contact time (i.e., incubation time) is that period of time that is sufficient to detect the presence of antibody to Mycobacterium tuberculosis within an infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. Detection reagent may then be added to the solid support. An appropriate detection reagent is any compound that binds to the immobilized antibody-polypeptide complex and that can be detected by any of a variety of means known to those in the art. The detection reagent generally contains a binding agent (such as, for example, Protein A, Protein G, immunoglobulin, lectin or free antigen) conjugated to a reporter group. Illustrative reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art.

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of Mycobacterium tuberculosis antibodies in a sample, the signal detected from the reporter group that remains bound to the solid support is generally compared to a signal that corresponds to a predetermined cut-off value. This cut-off value, for example, may be the average mean signal obtained when the immobilized antigen is incubated with samples from an uninfected patient. In certain embodiments, a sample generating a signal that is at least three standard deviations above the mean is considered positive for Mycobacterium tuberculosis antibodies and Mycobacterium tuberculosis infection. In another embodiment, the cut-off value may be determined using a Receiver Operator Curve, according to the method of Sackett et al., Clinical Epidemiology: A Basic Science for Clinical Medicine, p. 106-7 (Little Brown and Co., 1985). Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for Mycobacterium tuberculosis infection.

In other embodiments, an assay is performed in a flow-through assay format, wherein the antigen is immobilized on a membrane such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above.

In other embodiments, an assay if performed in a strip test format, such as a lateral flow assay format. For example, one end of the membrane to which polypeptide is bound is immersed in a solution containing the sample. The sample migrates along the membrane via capillary action through a region containing detection reagent and to the area of immobilized fusion polypeptide. Concentration of detection reagent at the fusion polypeptide indicates the presence of Leishmania antibodies in the sample. Typically, the concentration of detection reagent at that site generates a pattern, such as a line, that can be read visually. The absence of such a pattern indicates a negative result. In general, the amount of fusion polypeptide immobilized on the membrane is selected to generate a visually discernible pattern when the biological sample contains a level of antibodies that would be sufficient to generate a positive signal in an ELISA, as discussed above. Preferably, the amount of fusion polypeptide immobilized on the membrane ranges from about 25 ng to about 1 μg, and more preferably from about 50 ng to about 500 ng. Such tests can typically be performed with a very small amount (e.g., one drop) of patient serum or blood. Lateral flow tests can operate as either competitive or sandwich assays.

In still other embodiments, a fusion polypeptide of the invention is adapted for use in a dual path platform (DPP) assay. Such assays are described, for example, in U.S. Pat. No. 7,189,522, the contents of which are incorporated herein by reference.

In certain more specific embodiments, therefore, the invention provides a lateral flow or dual path platform diagnostic test device comprising at least three Mycobacterium tuberculosis seroreactive antigens, or immunogenic portions thereof, immobilized on a solid support, wherein the seroreactive antigens are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences.

In other more specific embodiments, there is provided a lateral flow or dual path platform diagnostic test device comprising a fusion polypeptide selected from the group consisting of DID90A (SEQ ID NO: 97), DID90B (SEQ ID NO: 98), DID104 (SEQ ID NO: 99), DID64 (SEQ ID NO: 100), DID65 (SEQ ID NO: 101), DID82 (SEQ ID NO: 102), DID96 (SEQ ID NO: 103), and DID94 (SEQ ID NO:104) or a sequence having at least 90% identity thereto, immobilized on a solid support.

Thus, in light of the present disclosure, it will be understood that the methods, kits and diagnostic reagents of the invention can use a fusion polypeptide or polypeptide combination in any of a variety of diagnostic assay formats known in the art, including, for example, a lateral flow test strip assay, a dual path platform (DPP) assay and an ELISA assay. The methods, kits and compositions of the invention can offer valuable point of care diagnostic information. Furthermore, the kits, compositions and methods herein can also be advantageously used in test-of-cure diagnostics for monitoring the status of infection in an infected individual over time and/or in response to treatment. Of course, numerous other assay protocols exist that are also suitable for use with the fusion polypeptides of the present invention. Accordingly, it will be understood that the above descriptions are intended to be exemplary only.

### D. Pharmaceutical and Vaccine Compositions

In another aspect, the present invention provides formulations of one or more of the polynucleotide, polypeptide or other compositions disclosed herein in pharmaceutically-acceptable or physiologically-acceptable solutions for administration to a cell or an animal, either alone, or in combination with one or more other modalities of therapy. Such pharmaceutical compositions are particularly preferred for use as vaccines when formulated with a suitable immunostimulant/adjuvant system. The compositions are also suitable for use in a diagnostic context.

It will also be understood that, if desired, the compositions of the invention may be administered in combination with other agents as well, such as, e.g., other proteins or polypeptides or various pharmaceutically-active agents. There is virtually no limit to other components that may also be included, provided that the additional agents do not cause a significant adverse effect upon the objectives according to the invention.

In certain preferred embodiments the compositions of the invention are used as vaccines and are formulated in combination with one or more immunostimulants. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see, e.g., Fullerton, U.S. Pat. No. 4,235,877). Vaccine preparation is generally described in, for example, Powell & Newman, eds., Vaccine Design (the subunit and adjuvant approach) (1995).

Any of a variety of immunostimulants may be employed in the vaccines of this invention. For example, an adjuvant may be included. Many adjuvants contain a substance designed to protect the antigen from rapid catabolism, such as aluminum hydroxide or mineral oil, and a stimulator of immune responses, such as lipid A (natural or synthetic), Bortadella pertussis or Mycobacterium species or Mycobacterium derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 and derivatives thereof (SmithKline Beecham, Philadelphia, Pa.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In certain preferred embodiments, the adjuvant used in the present invention is a glucopyranosyl lipid A (GLA) adjuvant, as described in pending U.S. patent application Ser. No. 11/862,122, the disclosure of which is incorporated herein by reference in its entirety. For example, certain GLA compounds of interest are represented by the following formula:

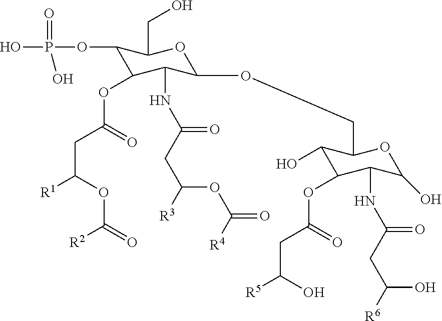

where: R¹, R³, R⁵ and R⁶ are C₁₁-C₂₀ alkyl; and R² and R⁴ are C₁₂-C₂₀ alkyl. In a more particular embodiment, R¹, R², R³, R⁴, R⁵ and R⁶ are C₁₄.

Other illustrative adjuvants useful in the context of the invention include Toll-like receptor agonists, such as TLR7 agonists, TLR7/8 agonists, and the like. Still other illustrative adjuvants include imiquimod (IMQ), gardiquimod (GDQ), resiquimod (RSQ), and related compounds.

Certain preferred vaccines employ adjuvant systems designed to induce an immune response predominantly of the Th1 type. High levels of Th1-type cytokines (e.g., IFN-γ, TNF, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mossman & Coffman, Ann. Rev. Immunol. 7:145-173 (1989).

Certain adjuvants for use in eliciting a predominantly Th1-type response include, for example, a combination of monophosphoryl lipid A, preferably 3-de-O-acylated monophosphoryl lipid A (3D-MPL™), together with an aluminum salt (U.S. Pat. Nos. 4,436,727; 4,877,611; 4,866,034; and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another illustrative adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other illustrative formulations include more than one saponin in the adjuvant combinations of the present invention, for example combinations of at least two of the following group comprising QS21, QS7, Quil A, escin, or digitonin.

In a particular embodiment, the adjuvant system includes the combination of a monophosphoryl lipid A and a saponin derivative, such as the combination of QS21 and 3D-MPL™ adjuvant, as described in WO 94/00153, or a less reactogenic composition where the QS21 is quenched with cholesterol, as described in WO 96/33739. Other formulations comprise an oil-in-water emulsion and tocopherol. Another adjuvant formulation employing QS21, 3D-MPL™ adjuvant and tocopherol in an oil-in-water emulsion is described in WO 95/17210.

Another enhanced adjuvant system involves the combination of a CpG-containing oligonucleotide and a saponin derivative as disclosed in WO 00/09159.

Other illustrative adjuvants include Montanide ISA 720 (Seppic, France), SAF (Novartis, Calif., United States), ISCOMS (CSL), MF-59 (Chiron), the SBAS series of adjuvants (e.g., SBAS-2, AS2′, AS2″, SBAS-4, or SBAS6, available from GlaxoSmithKline, Rixensart, Belgium), Detox, RC-529 (GlaxoSmithKline, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Compositions of the invention may also, or alternatively, comprise T cells specific for a Mycobacterium antigen. Such cells may generally be prepared in vitro or ex vivo, using standard procedures. For example, T cells may be isolated from bone marrow, peripheral blood, or a fraction of bone marrow or peripheral blood of a patient. Alternatively, T cells may be derived from related or unrelated humans, non-human mammals, cell lines or cultures.

T cells may be stimulated with a polypeptide of the invention, polynucleotide encoding such a polypeptide, and/or an antigen presenting cell (APC) that expresses such a polypeptide. Such stimulation is performed under conditions and for a time sufficient to permit the generation of T cells that are specific for the polypeptide. Preferably, the polypeptide or polynucleotide is present within a delivery vehicle, such as a microsphere, to facilitate the generation of specific T cells.

T cells are considered to be specific for a polypeptide of the invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070 (1994)). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a polypeptide of the invention (100 ng/ml-100 μg/ml, preferably 200 ng/ml-25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1 (1998)). T cells that have been activated in response to a polypeptide, polynucleotide or polypeptide-expressing APC may be CD4+ and/or CD8+. Protein-specific T cells may be expanded using standard techniques. Within preferred embodiments, the T cells are derived from a patient, a related donor or an unrelated donor, and are administered to the patient following stimulation and expansion.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intradermal, subcutaneous, and intramuscular administration and formulation.

In certain applications, the pharmaceutical compositions disclosed herein may be delivered via oral administration to a subject. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the pharmaceutical compositions disclosed herein parenterally, intravenously, intramuscularly, or even intraperitoneally as described, for example, in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the pharmaceutical compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

### EXAMPLES

### Example 1

### Identification of Mycobacterium Tuberculosis Antigens of High Serodiagnostic Value

This example describes the identification and characterization of combinations of Mycobacterium tuberculosis antigens that provide improved sensitivity and specificity in the diagnosis of tuberculosis.

### A. Materials and Methods

Study Populations.

Serum samples were obtained from individuals who had pulmonary tuberculosis prior to treatment (culture and/or acid fast bacteria (AFB) smear positive) previously obtained from Brazil (n=92) (Roberto Badaro, Federal University of Bahia, Salvador, Brazil) (Houghton et al., Clinical and diagnostic laboratory immunology 9:883-91, 2002). Serum samples obtained from India (sputum smear positive (n=36); sputum smear and culture negative endemic control (n=20)) were obtained from the World Health Organization TB Specimen Bank. Samples from healthy blood donors (n=46) were obtained from Boston Biomedica (West Bridgewater, Mass.). In all cases, drawing of blood was carried out with informed consent and the approval of the local ethics committee in the relevant country.

Antigen Identification, Cloning, and Purification.

Mtb genes were selected as previously described (J Immunol 181:7948-57, 2008). Briefly, Mtb genes included those previously identified by serological expression cloning and T-cell expression cloning methodologies (Methods in Molecular Medicine 94:91-106, 2004), those identified by proteomics as secreted or membrane associated by 2D PAGE and mass spectrometry analysis (www.mpiib-berlin.mpg.de/2D-PAGE/) (Electrophoresis 24:3405-20, 2003) or containing putative secretion signals, genes that were required for growth in macrophages (PNAS 100:12989-94, 2003), those that were up- or down-regulated in response to oxygen and carbon limitation (PNAS 98:7534-9, 2001), and mycobacterial specific genes within known immunogenic classes EsX and PE/PPE as based in Tuberculist (www.genolist. pasteur.fr/TubercuList/index.html). All targets were subjected to N-terminal signal sequence analysis and membrane spanning region using the SignalP (www.cbs.dtu.dk/services/SignalP/) and TMPred (www.ch.embnet.org/software/TMPRED_form.html) programs. Predicted proteins were chosen containing less than three transmembrane regions and a MW between 6-80 kDa.

DNA encoding selected Mtb genes was PCR amplified from HRv37 genomic DNA using Pfx DNA polymerase (Invitrogen, Carlsbad, Calif.). PCR primers were designed to incorporate specific restriction enzyme sites 5′ and 3′ of the gene of interest and excluded in the target gene for directional cloning into the expression vector pET17b or pET28a (Novagen, Madison, Wis.). After PCR amplification, purified PCR products were digested with restriction enzymes, ligated into pET28a using T4 DNA ligase (NEB), and transformed into XL10G cells (Stratagene). Recombinant plasmid DNA was recovered from individual colonies grown on LB agar plates containing appropriate antibiotics and sequenced to confirm the correctly cloned coding sequence. The recombinant clones contained an N-terminal six-histidine tag followed by thrombin cleavage site (pET28a) and the Mtb gene of interest.

Three fusion proteins (DID90A, DID90B, DID104) were designed to incorporate specific restriction enzyme sites 5′ and 3′ of the gene of interest with primer sequences as follows:

Rv0934mat-5′HindIII:
CAATTAAAGCTTT-GTGGCTCGAAACCACCGAGC
Rv0934-3′SacI:
CAATTAGAGCTCGCTGGAAAT-CGTCGCGATCAA
Rv2032-5′SacI:
CAATTAGAGCTCATGCCGGACACCATGGT-GACC
Rv2032-3′XhoI:
CAATTACTCGAGCTACCGGTGATCCTTAGCCCG
Rv2031-5′NdeI-6his:
CAATTACATATGCATCACCATCACCATCACATGGCCACCACCCTTCCCGTTC
Rv2031-3′HindIII:
CAATTAAGCTTGTTGGTGGACCGGATC-TGAATG
Rv2875mat-5′NdeI:
CAATTACATATGCATCACCATCACCATCACGGC-GATCTGGTGGGCCCG
Rv2875-3′HindIII:
CAATTAAAGCTTCGCCGGAGGCAT-TAGCACGCT
Rv0831-5′NdeI-6his:
CAGTTCCATATGCATCACCATCATCACCACATGCTCCCCGAGACAAATCAG
Rv0831-3′HindIII:
CTAGTCAAGCTTCTGGC-GAAGCAGCTCATCTTTC

The Rv0934 and Rv2032 genes were PCR amplified from pET plasmid template DNA (94° C. for 0:30; 58° C. for 0:30; 58° C. for 1:30; 30 cycles). Rv0934 was restriction enzyme digested with HindIII and SacI then cloned into the pET29a vector. The Rv2032PCR product was digested with SacI and XhoI and ligated into the pEt29a-Rv0934 vector to create pET29a-Rv0934-Rv2032. Rv2031 was digested with NdeI and HindIII and cloned into the pET29a-Rv0934-Rv2032 vector. The resulting plasmid was sequence verified as containing the fusion gene construct DID90A (Rv2031-Rv0934-Rv2032). The pET29a-DID90A plasmid encodes a 90 kDa protein containing an N-terminal six-histidine tag followed by the M. tuberculosis genes Rv2031, Rv0934 (C24-S374), and Rv2032 separated by restriction site linkers. The Rv2875mat PCR product was digested with NdeI and HindIII and ligated into digested pET29a-DID90A vector and sequence verified to generate pET29a-DID90B (Rv2875—Rv0934-Rv2032), encoding a 91 kDa protein containing an N-terminal six-histidine tag followed by the M. tuberculosis genes Rv2875 (G31-A193), Rv0934 (C24-S374), and Rv2032 separated by restriction site linkers. Rv0831 was digested with NdeI and HindIII and cloned into the digested pET29a-DID90A vector. The resulting plasmid was sequence verified as containing the fusion gene construct DID104 (Rv0831-Rv0934-Rv2032). The pET29a-DID104 plasmid encodes a 104 kDa protein containing an N-terminal six-histidine tag followed by the M. tuberculosis genes Rv0831, Rv0934 (C24-S374), and Rv2032 separated by restriction site linkers.

Recombinant plasmids were transformed into the E. coli BL21 derivative Rosetta²(DE3)(pLysS) (Novagen). Recombinant strains were cultured overnight at 37° C. in 2× yeast tryptone broth containing appropriate antibiotics, diluted 1/25 into fresh culture medium, grown to mid-log phase (optical density at 600 nm [OD600], 0.5 to 0.7), and induced by the addition of 1 mM IPTG. Cultures were grown for an additional 3 to 4 h, cells were harvested by centrifugation, and the bacterial pellets were stored at −20° C. Bacterial pellets were thawed and disrupted by sonication in 20 mM Tris (pH 8.0), 150 mM NaCl, 1 mM PMSF, followed by centrifugation to fractionate the soluble and insoluble material. Recombinant His-tagged protein products were isolated under native (soluble recombinant proteins) or denaturing (8M urea) conditions using Ni-nitrilotriacetic acid metal ion affinity chromatography according to the manufacturer's instructions (QIAGEN, Valencia, Calif.) followed by ion exchange chromatograghy (Biorad, Hercules, Calif.) when necessary. Protein fractions were eluted with an increasing imidazole gradient and analyzed by sodium dodecyl sulfate-polyacrylamide gel electrophoresis. Affinity-purified protein fractions were combined and dialyzed against 20 mM Tris, pH 8.0, concentrated using Amicon Ultra 10-kDa-molecular-mass cutoff centrifugal filters (Millipore, Billerica, Mass.), and quantified using the BCA protein assay (Pierce, Rockford, Ill.). LPS contamination was evaluated by the Limulus amoebocyte lysate assay (Cambrex Corp., East Rutherford, N.J.). All the recombinant proteins used in this study showed residual endotoxin levels below 100 EU/mg of protein.

Protein Array Serological Screening.

Glass-based chips were fabricated with duplicate sets of a total of 79 recombinant Mtb proteins (Full Moon Biosystems, Sunnyvale, Calif.). Human IgGl and EbaN1 were included as positive control proteins to verify array development, and buffer alone was included as a negative, background control. Sera were diluted 1/100 with blocking buffer and incubated with each slide at room temperature for 2 hours. After washing, slides were incubated with biotin-conjugated mouse anti-human IgG (H+L) (Jackson Immuno Research, West Grove, Pa.), washed and then developed with Cy5-conjugated streptavidin (Martek Biosciences, Columbia, Md.). Slides were scanned at 635 nm using GenePix Pro 6.0 (Molecular Devices, Sunnyvale, Calif.). The signal intensity of binding of each antigen for each individual serum was normalized versus the buffer alone spots for each individual serum to derive a fold-over-control (FOC) value. Data tables were statistically analyzed in MS Excel (Microsoft, Redmond, Wash.).

Antibody Detection by ELISA.

Polysorp 96-well plates (Nunc, Rochester, N.Y.) were coated with 50 ul of 2 μg/ml recombinant antigen in 0.1 M Sodium bicarbonate pH 9.6 overnight at 4° C. and then blocked for 2 hours at room temperature with PBST 1% (w/v) BSA on a plate shaker. Sera were diluted 1:100 in PBST 0.1% BSA in duplicate and added to each well. Plates were incubated at room temperature for 2 hours with shaking, then washed with PBST with 0.1% BSA and then HRP-conjugated IgG (Sigma, St. Louis, Mo.), diluted 1:10000 in PBST and 0.1% BSA, was added to each well and incubated at room temperature for 60 minutes with shaking. After washing, plates were developed with peroxidase color substrate (KPL, Baltimore Md.) with reaction quenched by addition of 1N H₂SO₄ after 15 minutes. The corrected optical density of each well at 450-570 nm was read using a VERSAmax® microplate reader (Molecular Devices, Sunnyvale, Calif.). Positive ELISA responses were defined as optical density readings exceeding 3-fold above the mean of the control sera, with a minimum defined optical density cut-off of 0.2.

Antigen Evaluation by MAPIA.

The assay was performed as previously described (Journal of immunological methods 242:91-100, 2000). Briefly, a semi-automatic micro-aerosolization device (Linomat IV; Camag Scientific Inc., Wilmington, Del.) was used to spray antigens at a range of concentrations between 0.02 mg/ml and 0.1 mg/ml through a syringe needle onto nitrocellulose membranes (Schleicher & Schuell, Inc., Keene, N.H.) to generate parallel bands. After antigen printing, the membrane was cut into strips 3 mm wide perpendicular to the antigen bands. The strips were blocked for 1 h with 1% nonfat milk in PBS containing 0.05% Tween 20 (PBST) and then incubated with individual serum samples diluted 1:50 in blocking solution for 1 h at room temperature. The strips were washed five times with PBST, and incubated for 1 h with alkaline phosphatase-conjugated anti-human IgG diluted 1:5,000 (Sigma, St. Louis, Mo.). The strips were washed with PBST as described above, and the human IgG antibodies bound to immobilized antigens were visualized with 5-bromo-4-chloro-3-indolylphosphate-nitroblue tetrazolium substrate (KPL). MAPIA results were scored by two independent operators who were unaware of the sample status. The appearance of any band of any intensity was read as a positive reaction.

### B. Results

Mtb Protein Array Screening for Seroreactivity.

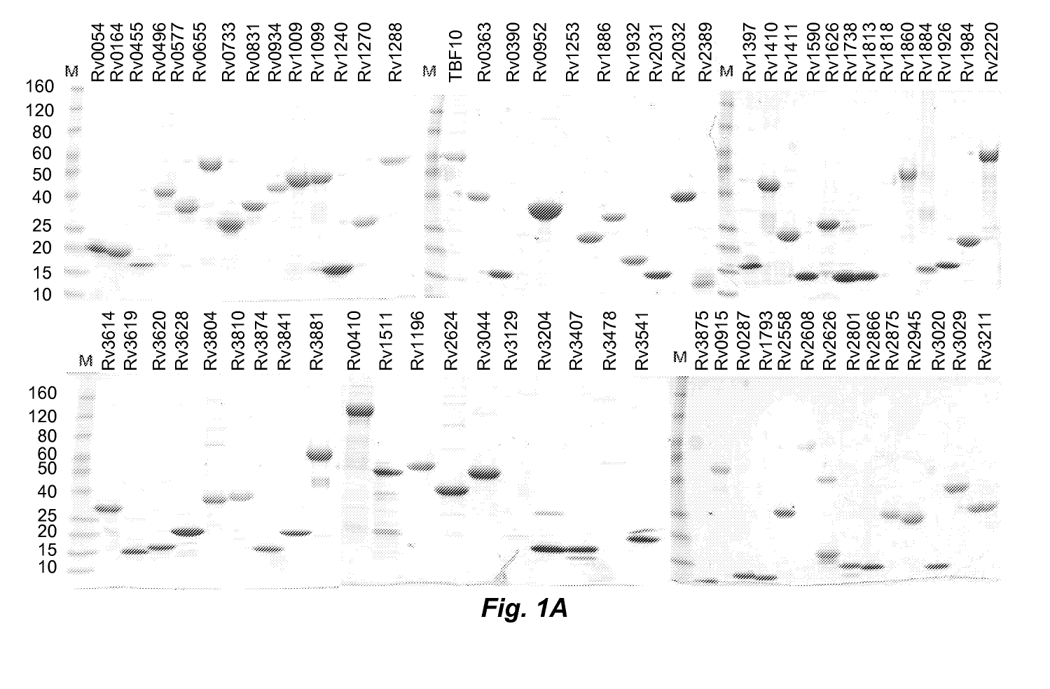

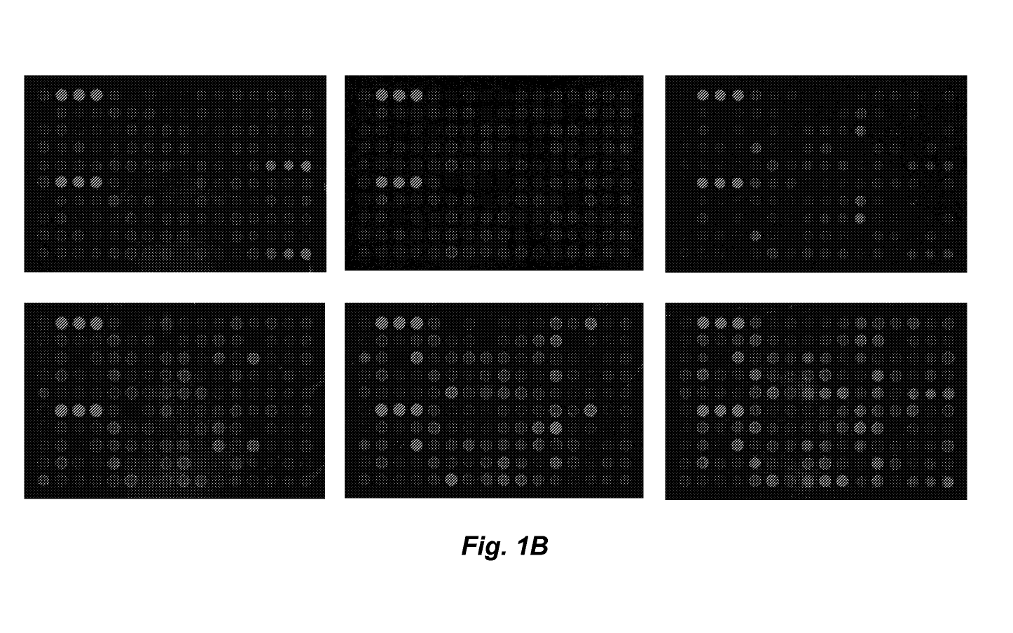

In previous work, we described the selection of a large body of Mtb antigens using data mining techniques to define new antigens with T-cell reactivity and vaccine potential (J Immunol 181:7948-57, 2008). In this study, we examined the humoral immune response to Mtb antigens by protein array and ELISA to identify antigens and antigen combinations with high diagnostic value. A total of 103 Mtb proteins were recombinantly produced in E. coli and with the majority achieving greater than 95% purity (FIG. 1A). Glass-based protein arrays were fabricated to comprehensively analyze the diagnostic potential of all antigens in a consistent and comparable fashion. A total of 79 Mtb proteins were expressed and immobilized in glass-based arrays and tested with 32 sera from sputum positive TB patients and 16 non-endemic (NEC) control sera. Several proteins were recognized and bound by IgG within sera samples, and could be grouped as a) TB-sensitive but lacking specificity (i.e. binding TB patient sera but also binding some NEC sera) and b) TB-specific (i.e. binding specific patient sera but not NEC sera). A total of 28 Mtb proteins displayed TB specific reactivity with a mean signal intensity of at least 3-fold above the controls (FIG. 1B and Table 1).

Mtb Antigen Characterization by ELISA.

To confirm protein array results and to test Mtb recombinant proteins not included on the protein arrays (n=24), ELISA screening was performed using the same serum set used for the protein arrays. 21 of the 28 antigens positive by protein array were also positive by ELISA. Antibody responses were observed for 6 proteins below the FOC=3 cutoff criteria by protein array, as well as with 9 additional proteins not present on the arrays (Table 1). A total of 42 proteins were found to bind antibodies in the sera of TB patients by either protein array or ELISA. These included 17 previously described immunogenic Mtb proteins: Rv0934 (38 Kd) (J Immunol 139:2447-51, 1987), Rv1813 (Vaccine 27:3063-71, 2009), Rv1827 (Cfp17) (FEMS Immunology and Medical Microbiology 23:159-64, 1999), Rv1837 (GlcB) (Journal of Clinical Microbiology 38:2354-61, 2000), Rv1860 (DPEP) (Int J Tuberc Lung Dis 4:377-83, 2000), Rv1886 (Ag85b) (Journal of Clinical Microbiology 29:2348-50, 1991), Rv1908 (katG) (Nature 358:591-3, 1992), Rv1984 (Infection and Immunity 66:3492-500, 1998), Rv2031 (a-crystallin) (Infection and Immunity 60:2066-74, 1992), Rv2220 (gInA1) (Infection and immunity 71:3927-36, 2003), Rv2608 (PPE42) (Journal of Infectious Diseases 190:1237-44, 2004), Rv2873 (mpt83) (Scandinavian Journal of Immunology 43:490-9, 1996), Rv2875 (mpt70) (Journal of Infectious Diseases 170:1326-30, 1994), Rv3407 (Infection and Immunity 72:6471-9, 2004), Rv3841 (Bfrb) (Mol Cell Proteomics 5:2102-13, 2006), Rv3874 (Cfp10) (Journal of Clinical Microbiology 38:3285-90, 2000), and Rv3881 (Mtb48) (Journal of clinical microbiology 39:2485-93, 2001); as well as 25 previously uncharacterized Mtb antigens (Rv0054, Rv0164, Rv0410, Rv0455, Rv0655, Rv0831, Rv0952, Rv1009, Rv1099, Rv1240, Rv1288, Rv1410, Rv1569, Rv1789, Rv2032, Rv2450, Rv2623, Rv2866, Rv3020, Rv3044, Rv3310, Rv3611, Rv3614, Rv3619, and Rv3628). The remainder of the recombinant antigens tested either failed to elicit significant antibody responses in this serum set, or showed non-specific binding with the control serum samples and therefore were excluded from further analysis.

TABLE 1

| | | Mol. | Immune | | Initial | Protein | ELISA |
|---|---|---|---|---|---|---|---|
| H37Rv | Gene | Mass | Function | Functional | Target | Array | Ab |
| Number | Name | (kDA) | (Reference) | Category | Selection | FOC | Response |
| Rv0054 | ssb | 17.3 | — | 2 | S | 4 | + |
| Rv0164 | TB18.5 | 17.7 | — | 10 | S | 3 | ++ |
| Rv0410 | pnkG | 81.6 | — | 9 | S | 3 | + |
| Rv0455c | Hyp | 16.6 | — | 10 | S | 1 | + |
| Rv0655 | mkl | 39.3 | — | 3 | M | 4 | ++ |
| Rv0831c | Hyp | 30.2 | — | 10 | S | 2 | +++ |
| Rv0934 | PstS1 | 38.2 | (18) | 3 | S | 4 | +++ |
| Rv0952 | sucD | 31.2 | — | 7 | B | 3 | − |
| Rv1009 | rpfB | 38 | | 3 | M | 4 | + |
| Rv1099 | Hyp | 34.6 | — | 10 | M | 3 | ++ |
| Rv1240 | mdh | 34.3 | — | 7 | H | n/d | + |
| Rv1288 | Hyp | 49.6 | — | 10 | B | 3 | + |
| Rv1410c | p55 | 54.7 | — | 3 | M | 3 | + |
| Rv1569 | bioF1 | 40 | — | 7 | M | n/d | ++ |
| Rv1789 | PPE26 | 38.6 | — | 6 | P/E | 3 | ++ |
| Rv1813c | Hyp | 15 | (5) | 10 | H | 3 | − |
| Rv1827 | cfp17 | 17.2 | (45) | 10 | EC | n/d | + |
| Rv1837 | Mtb81 | 80.7 | (15) | 10 | M | n/d | ++ |
| Rv1860 | apa | 32.7 | (9) | 3 | S | 3 | + |
| Rv1886c | fpbB | 34.6 | (44) | 1 | S | 3 | +++ |
| Rv1908 | katG | 80.6 | (48) | 0 | M | n/d | + |
| Rv1984c | cfp21 | 21.8 | (46) | 3 | S | 3 | −/+ |
| Rv2031 | acr | 16.2 | (21) | 0 | S | 3 | + |
| Rv2032 | acg | 36.6 | — | 10 | H | 5 | +++ |
| Rv2220 | glnA1 | 53.5 | (43) | 7 | S | 1 | ++ |
| Rv2450 | rpfE | 17.4 | — | 3 | B | 4 | −/+ |
| Rv2608 | PPE42 | 59.7 | (7) | 6 | P/E | 2 | +++ |
| Rv2623 | TB31.7 | 31.7 | — | 10 | H | 4 | + |
| Rv2866 | Hyp | 10.2 | — | 10 | H | 7 | + |
| Rv2873 | mpt83 | 20 | (16) | 3 | S | 3 | ++ |
| Rv2875 | mpt70 | 19.1 | (34) | 3 | S | 4 | +++ |
| Rv3020 | esxS | 9.8 | — | 3 | P/E | 3 | −/+ |
| Rv3044 | fecB | 36.9 | — | 3 | H | 1 | + |
| Rv3310 | SapM | 31.8 | — | 3 | S | n/d | ++ |
| Rv3407 | Hyp | 11 | (30) | 0 | B | 3 | −/+ |
| Rv3611 | Hyp | 23.8 | — | 16 | M | 3 | − |
| Rv3614 | Hyp | 19.8 | — | 10 | M | 5 | ++ |
| Rv3619 | esxI | 9.8 | — | 3 | P/E | 6 | + |
| Rv3628 | ppa | 18.3 | — | 7 | S | 3 | −/+ |
| Rv3841 | bfrB | 20.4 | (35) | 7 | EC | n/d | ++ |
| Rv3874 | Cfp10 | 10.8 | (11) | 3 | P/E | n/d | ++ |
| Rv3881 | Mtb48 | 47.6 | (25) | 10 | S | n/d | ++ |

In Table 1 above, the following designations apply: Functional classes as defined by Tuberculist: 0=virulence, detoxification, adaptation; 1=lipid metabolism; 2=information pathways; 3=cell wall and cell processes; 6=PE/PPE proteins; 7=intermediary metabolism; 8=unknown; 9=regulatory proteins; 10=conserved hypothetical; 16=conserved hypothetical with M. bovis ortholog (http://genolist.pasteur.fr/TubercuList/index.html). Selection Criteria: EC=expression cloning; S=secreted proteins; P/E=PE,PPE and EsX proteins; M=macrophage growth required; H=hypoxic response; B=other database searches. Protein Array FOC: mean fold increase of TB+ sera over normal control sera normalized against buffer controls. ELISA Ab Response: Positive ELISA responses were defined based on optical density readings exceeding 2-fold above the mean of the TB negative, non-endemic control panel sera with a minimum defined optical density cut-off of 0.2.

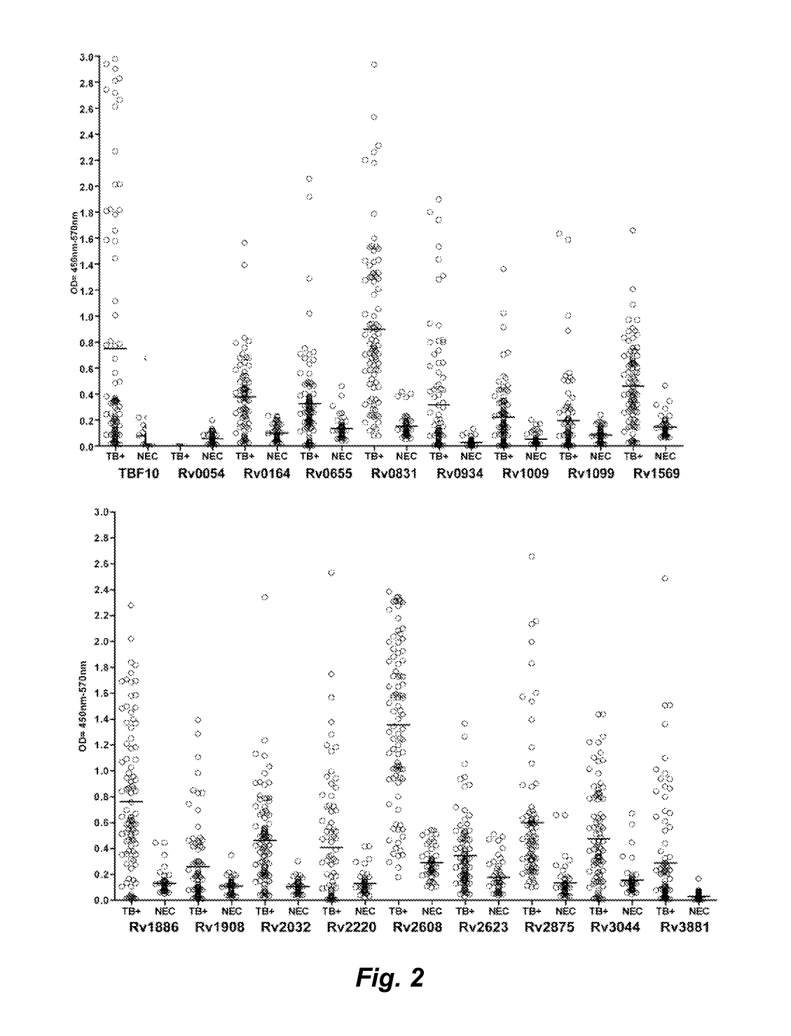

The antigens eliciting specific antibody responses on the initial screening by ELISA were further characterized on a larger panel of 92 serum samples of sputum-positive TB patients from Brazil and 46 control sera. The ELISA results are summarized in FIG. 2. TBF10, a previously characterized fusion consisting of three proteins (Rv0379, Rv0934, and Rv3874) was used as a reference antigen (Clinical and Diagnostic Laboratory Immunology 9:883-91, 2002). TBF10 detected antibody responses in 53 of the 92 TB sera (sensitivity 58%, specificity 89%). The recombinant antigens demonstrated variable sensitivities in ELISA ranging from 12% to 76%, with low or no reactivity with NEC sera (specificity 70-100%). Several antigens had individual sensitivities and specificities exceeding that of TBF10. These were Rv0831 (76%, 89%), Rv2875 (74%, 91%), Rv1886 (74%, 87%) and Rv2032 (70%, 96%). The Rv2608 antigen appeared to recognize a large proportion of the TB sera but had higher levels of background binding (specificity 70%). When antigen profiles to individual serum reactivity were analyzed, a combination of Rv2875, Rv2031, Rv2032, Rv0831, and TBF10 was able to detect antibody responses in 86 of the 92 TB samples (93% sensitivity), while 6 of 46 healthy control samples (87% specificity) reacted with one or more of these antigens. The 6 remaining TB samples failed to elicit antibody responses to any of the antigens or to a preparation of Mtb whole cell lysate (data not shown).

Designing Polyprotein Fusions.

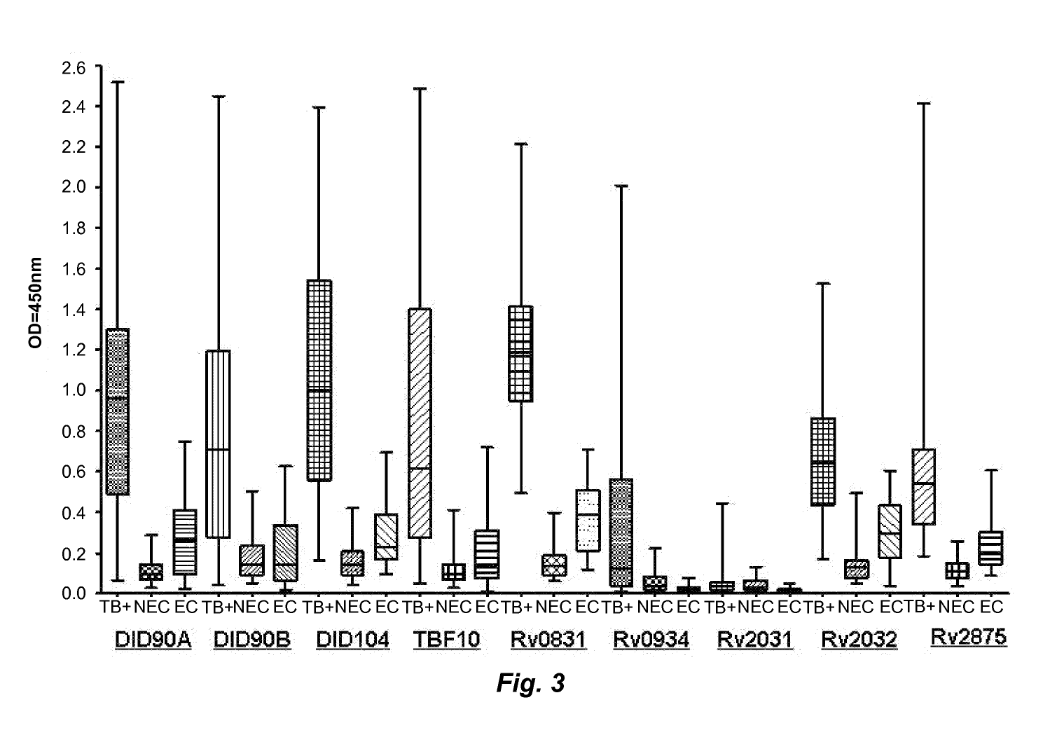

Due to the heterogeneity of the antibody response observed in TB patients, multiple antigens are necessary to increase the sensitivity of serodiagnostic tests. Based in the above ELISA antigen recognition patterns, we developed a series of fusion proteins designated DID90A (Rv2031-Rv0934-Rv2032), DID90B (Rv2875-Rv0934—Rv2032) and DID104 (Rv0831-Rv0934-Rv2032) to assess the ability of these antigens to complement each other when arranged in tandem. The antigen fusions and individual antigen components were assessed in ELISA using a panel of 36 TB sputum positive samples and 20 endemic controls (EC) from India, and compared to 20 NEC sera. As shown in FIG. 3, the DID90A and DID90B fusion proteins demonstrated reactivity profiles with the Indian TB and EC samples similar to that obtained for the TBF10 antigen (61% sensitivity, 85% specificity), with the DID104 fusion performing slightly better (69% sensitivity, 85% specificity). Some differences were observed among the recognition of the individual antigens within the Brazilian and Indian serum cohorts. Among the Indian Tb+ samples, Rv0831 had increased sensitivity (83%) but also cross-reacted with the endemic control sera (70% sensitivity). Rv2875 (55%, 90%) and Rv2032 (53%, 85%) had a slight decrease in sensitivity but with similar specificities. The Rv0934 antigen exhibited a similar reactivity profile in both Brazilian and Indian cohorts (41% sensitivity, 95% specificity) while Rv2031 was poorly recognized among these serum samples.

Characterization of Mtb Antigens by MAPIA.

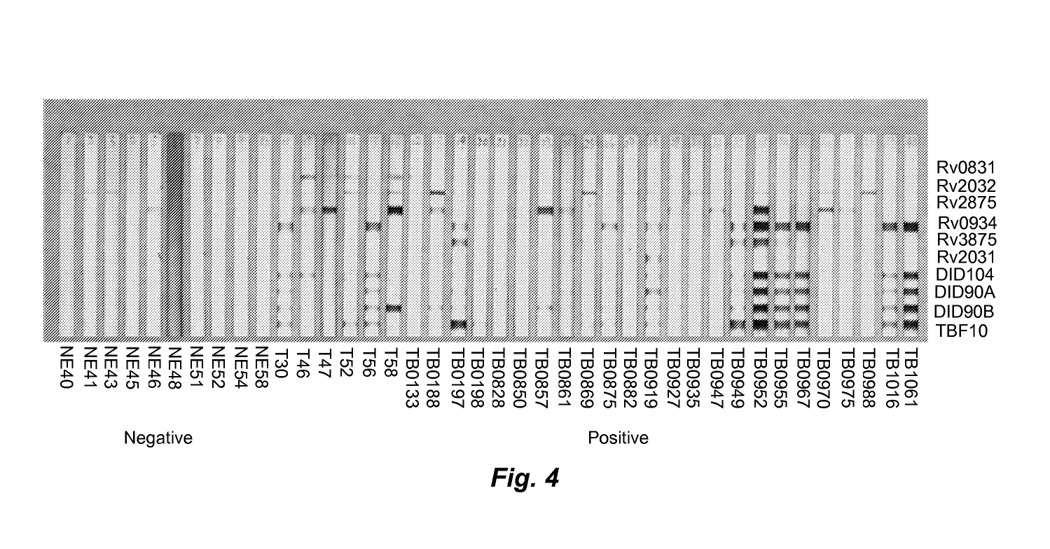

We used MAPIA to further validate the selected antigens and fusion molecules most suitable for developing rapid lateral-flow assays. MAPIA involves the immobilization of multiple antigens on nitrocellulose membranes and provides a valuable means to characterize individual recognition patterns. We have previously found that serological performance of antigens in MAPIA is a good predictor of their performance in other membrane-based assays (Journal of Immunological Methods 242:91-100, 2000). The four fusion proteins along with the single component antigens were evaluated by MAPIA. FIG. 4 demonstrates the presence of IgG antibodies in most TB sera against several single antigens and fusion proteins. Antibody responses to at least one antigen could be detected in 27/30 TB serum samples, while no or very weak bands were observed in the negative control group.

### C. Discussion

It has been suggested that implementation of rapid serological tests would be useful in combination with other methods for diagnosis of active TB in settings where bacterial culture is not routinely available (Am J Respir Crit. Care Med 162:1323-9, 2000). However, so far none of the rapid serodiagnostics has proven reproducibly accurate, preventing their widespread application. Antibody responses in TB are directed against a broad set of antigens, with remarkable patient-to-patient variation of antigen recognition (Scand J Immunol 66:176-91, 2007). Even with taking this variation into account, the sensitivities have generally been poor (Scand J Immunol 66:176-91, 2007; Lancet 356:1099-104, 2000; Infect Immun 66:3936-40, 1998). The low specificities in antibody-based tests evaluated to date may result from the presence of antibodies to any of the following circumstances: latent TB infection, inactive (treated) disease, prior vaccination with Mycobacterium bovis bacillus Calmette-Guerin (BCG), or exposure to non-TB mycobacteria. Since these conditions may influence performance of serological assays, reported results that were obtained in different clinical settings vary significantly (Arch Intern Med 163:1009-21, 2003), with test sensitivities ranging from 10-90% and specificities ranging from 47-100% (J Clin Microbiol 38:2227-31, 2000; PLoS Med 4:e202, 2007; Future Microbiol 2:355-9, 2007). The higher test sensitivities are typically associated with the lower specificities, and vice versa; no commercial serologic test is currently available that meets an acceptable level of sensitivity and specificity (Future Microbiol 2:355-9, 2007). Despite these limitations, the interest in developing simple formats for rapid TB diagnosis remains high for field implementation in resource limited settings.

We expressed and purified over 100 potential Mtb proteins selected from genome and database mining. The present study examined the serodiagnostic value of the candidate molecules by protein array, ELISA, and MAPIA. As expected, many of the proteins were nonreactive with TB patient sera, while others reacted with both TB patient and control sera. Such proteins were excluded from further analyses. From the initial protein array and ELISA screens, 42 antigens demonstrated various degrees of reactivity with TB patient sera. Among these, 17 antigens were previously reported, while the remaining 25 proteins appeared to be previously uncharacterized. These antigens included 16 presumptively secreted or membrane associated antigens, 8 antigens based on genes required for growth in macrophages, 6 antigens induced by hypoxia, 5 antigens associated with virulence from the PE/PPE and EsX classes, and 4 from other database searches. While there was generally good concordance between the assays with 21 of 28 proteins positive for specific TB seroreactivity, some differences were observed. Seven antigens positive by protein array (Rv0952, Rv1813, Rv1984, Rv2450, Rv3020, and Rv3407) showed very low or no responses by ELISA; conversely, 5 proteins positive by ELISA (Rv0455, Rv0831, Rv2220, Rv2608, and Rv3044) failed to demonstrate significant responses in protein arrays. These discrepancies may be due to variable coating efficiencies of antigens or to differences between assays in calculating cut-off values.

The seroreactive TB antigens were analyzed for responses on a larger panel of TB serum samples from sputum positive patients and NEC sera to further reduce the antigen complexity down to those most useful at diagnosing active TB. The antigens demonstrated variable individual sensitivities ranging from 12% to 78%, with generally low background binding (specificity ˜76-100%). Typically, antigens with low sensitivities had higher specificities (Rv1860 12%, 100%; Rv3874 16%, 100%), while increasing sensitivity resulted in decreased specificity (Rv2608, 78%, 76%; Rv1886, 74%, 87%). Based on additive responses among individual serum samples, Rv0934, Rv3874, Rv2875, Rv2031, Rv2032, and Rv0831 defined a minimal subset of illustrative antigens for providing the greatest overall sensitivity. When these seroreactive antigens were analyzed in combinations, 93% of antibody responders could be identified among the TB patients. A number of the antigens described (Rv0455, Rv3619, Rv3310, Rv1410, Rv1240) had redundant patterns of reactivity with other antigens and therefore they could not increase the overall sensitivity.

The generation of fusion proteins has been used as a means to reduce the cost and complexity of antigen cocktails in rapid lateral-flow formats and increase sensitivity and specificity (Clinical and Diagnostic Laboratory Immunology 9:883-91, 2002; Clin Vaccine Immunol 16:260-76, 2009). We generated a series of related fusion proteins and tested them in ELISA along with the individual antigen components. The three new fusions demonstrated similar sensitivities and specificities with a serum panel from India and were comparable to the reference antigen TBF10. MAPIA using the fusion antigens and selected individual components also demonstrated that the vast majority of the TB patients (90%) produced antibody responses to one or more antigens, with a combination of 6 proteins (Rv0831, Rv2031, Rv2032, Rv2875, Rv0934, and Rv3874) providing the greatest sensitivity.

The remarkable variation in the immune recognition patterns in TB requires multi-antigen cocktails to cover the heterogeneity of antibody responses and thus achieve the highest possible test sensitivity. Such antigen cocktails and/or the production of fusion molecules comprised of antigens described herein provide improved sensitivity and specificity for the development of a rapid, accurate, and inexpensive point-of-care diagnostic test.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A diagnostic composition comprising a combination of three or more Mycobacterium tuberculosis seroreactive antigens, or immunogenic fragments thereof, wherein the antigens are selected from the group consisting of Rv0054 (SEQ ID NO: 1), Rv0164 (SEQ ID NO: 3), Rv0410 (SEQ ID NO: 5), Rv0455c (SEQ ID NO: 7), Rv0632 (SEQ ID NO: 9) Rv0655 (SEQ ID NO: 11), Rv0831c (SEQ ID NO: 13), Rv0934 (SEQ ID NO: 15), Rv0952 (SEQ ID NO: 17), Rv1009 (SEQ ID NO: 19), Rv1099 (SEQ ID NO: 21), Rv1240 (SEQ ID NO: 23), Rv1288 (SEQ ID NO: 25), Rv1410c (SEQ ID NO: 27), ), Rv1411 (SEQ ID NO: 29) Rv1569 (SEQ ID NO: 31), Rv1789 (SEQ ID NO: 33), Rv1813c (SEQ ID NO: 35), Rv1827 (SEQ ID NO: 37), Rv1837 (SEQ ID NO: 39), Rv1860 (SEQ ID NO: 41), Rv1886c (SEQ ID NO: 43), Rv1908 (SEQ ID NO: 45), Rv1980 (SEQ ID NO:47), Rv1984c (SEQ ID NO: 49), Rv2031 (SEQ ID NO: 51), Rv2032 (SEQ ID NO: 53), Rv2220 (SEQ ID NO: 55), Rv2450 (SEQ ID NO: 57), Rv2608 (SEQ ID NO: 59), Rv2623 (SEQ ID NO: 61), Rv2866 (SEQ ID NO: 63), Rv2873 (SEQ ID NO: 65), Rv2875 (SEQ ID NO: 67), Rv3020 (SEQ ID NO: 69), Rv3044 (SEQ ID NO: 71), Rv3310 (SEQ ID NO: 73), Rv3407 (SEQ ID NO: 75), Rv3611 (SEQ ID NO: 77), Rv3614 (SEQ ID NO: 79), Rv3616 (SEQ ID NO: 81) Rv3619 (SEQ ID NO: 83), Rv3628 (SEQ ID NO: 85), Rv3804 (SEQ ID NO:87), Rv3841 (SEQ ID NO: 89), Rv3864 (SEQ ID NO:91), Rv3874 (SEQ ID NO: 93) and Rv3881 (SEQ ID NO: 95), and antigens having at least 90% identity to any of the foregoing sequences.

2. The diagnostic composition of claim 1, wherein the seroreactive antigens are selected from the group consisting of Rv0455 (SEQ ID NO: 7), Rv0632 (SEQ ID NO:9), Rv0831 (SEQ ID NO: 13), Rv0934 (SEQ ID NO: 15), ), Rv1240 (SEQ ID NO: 23), Rv1410 (SEQ ID NO: 27), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3310 (SEQ ID NO: 73), ), Rv3619 (SEQ ID NO: 83), Rv3864 (SEQ ID NO:91), Rv3874 (SEQ ID NO: 93), Rv3881 (SEQ ID NO: 95), and antigens having at least 90% identity to any of the foregoing sequences.

3. The diagnostic composition of claim 1, wherein the seroreactive antigens are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences.

4. The diagnostic composition of claim 1, wherein seroreactive antigens, or immunogenic fragments thereof, are covalently linked in the form of a fusion polypeptide.

5. The diagnostic composition of claim 4, wherein the fusion polypeptide comprises an amino acid sequence selected from the group consisting of DID90A (SEQ ID NO: 97), DID90B (SEQ ID NO: 98), DID104 (SEQ ID NO: 99), DID64 (SEQ ID NO: 100), DID65 (SEQ ID NO:101), DID82 (SEQ ID NO: 102), DID96 (SEQ ID NO:103) and DID94 (SEQ ID NO: 104) or a sequence having at least 90% identity thereto.

6. (canceled)

7. (canceled)

8. An isolated fusion polypeptide comprising a combination of three or more covalently linked Mycobacterium tuberculosis seroreactive antigens, or immunogenic fragments thereof, wherein the antigens are selected from the group consisting of Rv0054 (SEQ ID NO: 1), Rv0164 (SEQ ID NO: 3), Rv0410 (SEQ ID NO: 5), Rv0455c (SEQ ID NO: 7), Rv0632 (SEQ ID NO: 9) Rv0655 (SEQ ID NO: 11), Rv0831c (SEQ ID NO: 13), Rv0934 (SEQ ID NO: 15), Rv0952 (SEQ ID NO: 17), Rv1009 (SEQ ID NO: 19), Rv1099 (SEQ ID NO: 21), Rv1240 (SEQ ID NO: 23), Rv1288 (SEQ ID NO: 25), Rv1410c (SEQ ID NO: 27), ), Rv1411 (SEQ ID NO: 29) Rv1569 (SEQ ID NO: 31), Rv1789 (SEQ ID NO: 33), Rv1813c (SEQ ID NO: 35), Rv1827 (SEQ ID NO: 37), Rv1837 (SEQ ID NO: 39), Rv1860 (SEQ ID NO: 41), Rv1886c (SEQ ID NO: 43), Rv1908 (SEQ ID NO: 45), Rv1980 (SEQ ID NO:47), Rv1984c (SEQ ID NO: 49), Rv2031 (SEQ ID NO: 51), Rv2032 (SEQ ID NO: 53), Rv2220 (SEQ ID NO: 55), Rv2450 (SEQ ID NO: 57), Rv2608 (SEQ ID NO: 59), Rv2623 (SEQ ID NO: 61), Rv2866 (SEQ ID NO: 63), Rv2873 (SEQ ID NO: 65), Rv2875 (SEQ ID NO: 67), Rv3020 (SEQ ID NO: 69), Rv3044 (SEQ ID NO: 71), Rv3310 (SEQ ID NO: 73), Rv3407 (SEQ ID NO: 75), Rv3611 (SEQ ID NO: 77), Rv3614 (SEQ ID NO: 79), Rv3616 (SEQ ID NO: 81) Rv3619 (SEQ ID NO: 83), Rv3628 (SEQ ID NO: 85), Rv3804 (SEQ ID NO:87), Rv3841 (SEQ ID NO: 89), Rv3864 (SEQ ID NO:91), Rv3874 (SEQ ID NO: 93) and Rv3881 (SEQ ID NO: 95), and antigens having at least 90% identity to any of the foregoing sequences.

9. The isolated fusion polypeptide of claim 8, wherein the seroreactive antigens are selected from the group consisting of Rv0455 (SEQ ID NO: 7), Rv0632 (SEQ ID NO:9), Rv0831 (SEQ ID NO: 13), Rv0934 (SEQ ID NO: 15), ), Rv1240 (SEQ ID NO: 23), Rv1410 (SEQ ID NO: 27), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3310 (SEQ ID NO: 73), ), Rv3619 (SEQ ID NO: 83), Rv3864 (SEQ ID NO:91), Rv3874 (SEQ ID NO: 93), Rv3881 (SEQ ID NO: 95), and antigens having at least 90% identity to any of the foregoing sequences.

10. The isolated fusion polypeptide of claim 8, wherein the seroreactive antigens are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences.

11-26. (canceled)

27. An isolated polynucleotide encoding a fusion polypeptide of claim 8.

28. A method for detecting Mycobacterium tuberculosis in a biological sample, comprising
(a) contacting the biological sample with a combination of three or more Mycobacterium tuberculosis seroreactive antigens, or immunogenic fragments thereof, wherein the antigens are selected from the group consisting of Rv0054 (SEQ ID NO: 1), Rv0164 (SEQ ID NO: 3), Rv0410 (SEQ ID NO: 5), Rv0455c (SEQ ID NO: 7), Rv0632 (SEQ ID NO: 9) Rv0655 (SEQ ID NO: 11), Rv0831c (SEQ ID NO: 13), Rv0934 (SEQ ID NO: 15), Rv0952 (SEQ ID NO: 17), Rv1009 (SEQ ID NO: 19), Rv1099 (SEQ ID NO: 21), Rv1240 (SEQ ID NO: 23), Rv1288 (SEQ ID NO: 25), Rv1410c (SEQ ID NO: 27), ), Rv1411 (SEQ ID NO: 29) Rv1569 (SEQ ID NO: 31), Rv1789 (SEQ ID NO: 33), Rv1813c (SEQ ID NO: 35), Rv1827 (SEQ ID NO: 37), Rv1837 (SEQ ID NO: 39), Rv1860 (SEQ ID NO: 41), Rv1886c (SEQ ID NO: 43), Rv1908 (SEQ ID NO: 45), Rv1980 (SEQ ID NO:47), Rv1984c (SEQ ID NO: 49), Rv2031 (SEQ ID NO: 51), Rv2032 (SEQ ID NO: 53), Rv2220 (SEQ ID NO: 55), Rv2450 (SEQ ID NO: 57), Rv2608 (SEQ ID NO: 59), Rv2623 (SEQ ID NO: 61), Rv2866 (SEQ ID NO: 63), Rv2873 (SEQ ID NO: 65), Rv2875 (SEQ ID NO: 67), Rv3020 (SEQ ID NO: 69), Rv3044 (SEQ ID NO: 71), Rv3310 (SEQ ID NO: 73), Rv3407 (SEQ ID NO: 75), Rv3611 (SEQ ID NO: 77), Rv3614 (SEQ ID NO: 79), Rv3616 (SEQ ID NO: 81) Rv3619 (SEQ ID NO: 83), Rv3628 (SEQ ID NO: 85), Rv3804 (SEQ ID NO:87), Rv3841 (SEQ ID NO: 89), Rv3864 (SEQ ID NO:91), Rv3874 (SEQ ID NO: 93) and Rv3881 (SEQ ID NO: 95), and antigens having at least 90% identity to any of the foregoing sequences; and
(b) detecting in the biological sample the presence of antibodies that bind thereto.

29. (canceled)

30. (canceled)

31. The method of claim 28, wherein seroreactive antigens, or immunogenic fragments thereof, are covalently linked in the form of a fusion polypeptide.

32. The method of claim 31, wherein the fusion polypeptide comprises an amino acid sequence selected from the group consisting of DID90A (SEQ ID NO: 97), DID90B (SEQ ID NO: 98), DID104 (SEQ ID NO: 99), DID64 (SEQ ID NO: 100), DID65 (SEQ ID NO: 101), DID82 (SEQ ID NO: 102), DID96 (SEQ ID NO: 103), and DID94 (SEQ ID NO:104) or a sequence having at least 90% identity thereto.

33. (canceled)

34. (canceled)

35. The method of claim 33, wherein the method is carried out in an assay format selected from the group consisting of an ELISA assay, a lateral flow strip test assay and a dual path platform assay.

36. The method of claim 33, wherein the method is a test-of-cure method for monitoring the status of infection in an infected individual over time or in response to treatment.

37. A diagnostic kit for detecting Mycobacterium tuberculosis infection in a biological sample, comprising:
(a) a combination of three or more Mycobacterium tuberculosis seroreactive antigens, or immunogenic fragments thereof, wherein the antigens are selected from the group consisting of Rv0054 (SEQ ID NO: 1), Rv0164 (SEQ ID NO: 3), Rv0410 (SEQ ID NO: 5), Rv0455c (SEQ ID NO: 7), Rv0632 (SEQ ID NO: 9) Rv0655 (SEQ ID NO: 11), Rv0831c (SEQ ID NO: 13), Rv0934 (SEQ ID NO: 15), Rv0952 (SEQ ID NO: 17), Rv1009 (SEQ ID NO: 19), Rv1099 (SEQ ID NO: 21), Rv1240 (SEQ ID NO: 23), Rv1288 (SEQ ID NO: 25), Rv1410c (SEQ ID NO: 27), ), Rv1411 (SEQ ID NO: 29) Rv1569 (SEQ ID NO: 31), Rv1789 (SEQ ID NO: 33), Rv1813c (SEQ ID NO: 35), Rv1827 (SEQ ID NO: 37), Rv1837 (SEQ ID NO: 39), Rv1860 (SEQ ID NO: 41), Rv1886c (SEQ ID NO: 43), Rv1908 (SEQ ID NO: 45), Rv1980 (SEQ ID NO:47), Rv1984c (SEQ ID NO: 49), Rv2031 (SEQ ID NO: 51), Rv2032 (SEQ ID NO: 53), Rv2220 (SEQ ID NO: 55), Rv2450 (SEQ ID NO: 57), Rv2608 (SEQ ID NO: 59), Rv2623 (SEQ ID NO: 61), Rv2866 (SEQ ID NO: 63), Rv2873 (SEQ ID NO: 65), Rv2875 (SEQ ID NO: 67), Rv3020 (SEQ ID NO: 69), Rv3044 (SEQ ID NO: 71), Rv3310 (SEQ ID NO: 73), Rv3407 (SEQ ID NO: 75), Rv3611 (SEQ ID NO: 77), Rv3614 (SEQ ID NO: 79), Rv3616 (SEQ ID NO: 81) Rv3619 (SEQ ID NO: 83), Rv3628 (SEQ ID NO: 85), Rv3804 (SEQ ID NO:87), Rv3841 (SEQ ID NO: 89), Rv3864 (SEQ ID NO:91), Rv3874 (SEQ ID NO: 93) and Rv3881 (SEQ ID NO: 95), and antigens having at least 90% identity to any of the foregoing sequences; and
(b) a detection reagent.

38. The diagnostic kit of claim 37, wherein the seroreactive antigens are selected from the group consisting of Rv0455 (SEQ ID NO: 7), Rv0632 (SEQ ID NO:9), Rv0831 (SEQ ID NO: 13), Rv0934 (SEQ ID NO: 15), ), Rv1240 (SEQ ID NO: 23), Rv1410 (SEQ ID NO: 27), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3310 (SEQ ID NO: 73), ), Rv3619 (SEQ ID NO: 83), Rv3864 (SEQ ID NO:91), Rv3874 (SEQ ID NO: 93), Rv3881 (SEQ ID NO: 95), and antigens having at least 90% identity to any of the foregoing sequences.

39. (canceled)

40. The diagnostic kit of claim 37, wherein seroreactive antigens, or immunogenic fragments thereof, are covalently linked in the form of a fusion polypeptide.

41. The diagnostic kit of claim 40, wherein the fusion polypeptide comprises an amino acid sequence selected from the group consisting of DID90A (SEQ ID NO: 97), DID90B (SEQ ID NO: 98), DID104 (SEQ ID NO: 99), DID64 (SEQ ID NO: 100), DID65 (SEQ ID NO: 101), DID82 (SEQ ID NO: 102), DID96 (SEQ ID NO: 103), and DID94 (SEQ ID NO:104) or a sequence having at least 90% identity thereto.

42. (canceled)

43. (canceled)

44. A lateral flow or dual path platform diagnostic test device comprising at least three Mycobacterium tuberculosis seroreactive antigens, or immunogenic portions thereof, immobilized on a solid support, wherein the seroreactive antigens are selected from the group consisting of Rv0632 (SEQ ID NO:9), Rv0831 (SEQ ID NO: 13), Rv0934 (SEQ ID NO: 15), Rv1860 (SEQ ID NO: 41), Rv1980 (SEQ ID NO:47), Rv2031 (SEQ ID NO: 51), Rv2032 (SEQ ID NO: 53), Rv2875 (SEQ ID NO: 67), Rv3864 (SEQ ID NO:91), Rv3874 (SEQ ID NO: 93), Rv3881 (SEQ ID NO: 95), and antigens having at least 90% identity to any of the foregoing sequences.

45. A lateral flow or dual path platform diagnostic test device comprising a fusion polypeptide selected from the group consisting of DID90A (SEQ ID NO: 97), DID90B (SEQ ID NO: 98), DID104 (SEQ ID NO: 99), DID64 (SEQ ID NO: 100), DID65 (SEQ ID NO: 101), pa-1593124 DID82 (SEQ ID NO: 102), DID96 (SEQ ID NO: 103), and DID94 (SEQ ID NO:104) or a sequence having at least 90% identity thereto, immobilized on a solid support.


SEQUENCE LISTING (104 sequences)

SEQ ID NO: 1 - protein, 184 aa, Mycobacterium tuberculosis
MGSSHHHHHHSSGLVPRGSHMAGDTTITIVGNLTADPELRFTPSGAAVANFTVASTPRIYDRQTGEWKDGEALFLRCNIWREAAENVAESLTRGARVIVSGRLKQRSFETREGEKRTVIEVEVDEIGPSLRYATAKVNKASRSGGFGSGSRPAPAQTSSASGDDPWGSAPASGSFGGGDDEPPF

SEQ ID NO: 2 - DNA, 561 bp, Mycobacterium tuberculosis
atgggcagcagccatcatcatcatcatcacagcagcggcctggtgccgcgcggcagccatatggctggtgacaccaccatcaccatcgtcggaaatctgaccgctgaccccgagctgcggttcaccccgtccggtgcggccgtggcgaatttcaccgtggcgtcaacgccccggatctatgaccgtcagaccggcgaatggaaagacggcgaagcgctgttcctccggtgcaatatctggcgggaggcggccgagaacgtggccgagagcctcacccggggggcacgagtcatcgttagcgggcggcttaagcagcggtcgtttgaaacccgtgagggcgagaagcgcaccgtcatcgaggtcgaggtcgatgagattgggccttcgcttcggtacgccaccgccaaggtcaacaaggccagccgcagcggcgggtttggcagcggatcccgtccggcgccggcgcagaccagcagcgcctcgggagatgacccgtggggcagcgcaccggcgtcgggttcgttcggcggcggcgatgacgaaccgccattctgaaagctt

SEQ ID NO: 3 - protein, 181 aa, Mycobacterium tuberculosis
MGSSHHHHHHSSGLVPRGSHMTAISCSPRPRYASRMPVLSKTVEVTADAASIMAIVADIERYPEWNEGVKGAWVLARYDDGRPSQVRLDTAVQGIEGTYIHAVYYPGENQIQTVMQQGELFAKQEQLFSVVATGAASLLTVDMDVQVTMPVPEPMVKMLLNNVLEHLAENLKQRAEQLAAS

SEQ ID NO: 4 - DNA, 486 bp, Mycobacterium tuberculosis
atgacggcaatctcgtgctcaccgcgacccaggtatgcttcccgaatgccagttttgagcaagaccgtcgaggtcaccgccgacgccgcatcgatcatggccatcgttgccgatatcgagcgctacccagagtggaatgaaggggtcaagggcgcatgggtgctcgctcgctacgatgacgggcgtcccagccaggtgcggctcgacaccgctgttcaaggcatcgagggcacctatatccacgccgtgtactacccaggcgaaaaccagattcaaaccgtcatgcagcagggtgaactgtttgccaagcaggagcagctgttcagtgtggtggcaaccggcgccgcgagcttgctcacggtggacatggacgtccaggtcaccatgccggtgcccgagccgatggtgaagatgctgctcaacaacgtcctggagcatctcgccgaaaatctcaagcagcgcgccgagcagctggcggccagctaa

SEQ ID NO: 5 - protein, 757 aa, Mycobacterium tuberculosis
MHHHHHHMAKASETERSGPGTQPADAQTATSATVRPLSTQAVFRPDFGDEDNFPHPTLGPDTEPQDRMATTSRVRPPVRRLGGGLVEIPRAPDIDPLEALMTNPVVPESKRFCWNCGRPVGRSDSETKGASEGWCPYCGSPYSFLPQLNPGDIVAGQYEVKGCIAHGGLGWIYLALDRNVNGRPVVLKGLVHSGDAEAQAMAMAERQFLAEVVHPSIVQIFNFVEHTDRHGDPVGYIVMEYVGGQSLKRSKGQKLPVAEAIAYLLEILPALSYLHSIGLVYNDLKPENIMLTEEQLKLIDLGAVSRINSFGYLYGTPGFQAPEIVRTGPTVATDIYTVGRTLAALTLDLPTRNGRYVDGLPEDDPVLKTYDSYGRLLRRAIDPDPRQRFTTAEEMSAQLTGVLREVVAQDTGVPRPGLSTIFSPSRSTFGVDLLVAHTDVYLDGQVHAEKLTANEIVTALSVPLVDPTDVAASVLQATVLSQPVQTLDSLRAARHGALDADGVDFSESVELPLMEVRALLDLGDVAKATRKLDDLAERVGWRWRLVWYRAVAELLTGDYDSATKHFTEVLDTFPGELAPKLALAATAELAGNTDEHKFYQTVWSTNDGVISAAFGLARARSAEGDRVGAVRTLDEVPPTSRHFTTARLTSAVTLLSGRSTSEVTEEQIRDAARRVEALPPTEPRVLQIRALVLGGALDWLKDNKASTNHILGFPFTSHGLRLGVEASLRSLARVAPTQRHRYTLVDMANKVRPTSTF

SEQ ID NO: 6 - DNA, 2275 bp, Mycobacterium tuberculosis
atgcatcatacatcatcatcatatggccaaagcgtcagagaccgaacgttcgggccccggcacccaaccggcggacgcccagaccgcgacgtccgcgacggttcgacccctgagcacccaggcggtgttccgccccgatttcggcgatgaggacaacttcccccatccgacgctcggcccggacaccgagccgcaagaccggatggccaccaccagccgggtgcgcccgccggtcagacggctgggcggcggcctggtggaaatcccgcgggcgcccgatatcgatccgcttgaggccctgatgaccaacccggtggtgccggagtccaagcggttctgctggaactgtggacgtcccgtcggccggtccgactcggagaccaagggagcttcagagggctggtgtccctattgcggcagcccgtattcgttcctgccgcagctaaatcccggggacatcgtcgccggccagtacgaggtcaaaggctgcatcgcgcacggcggactgggctggatctacctcgctctcgaccgcaatgtcaacggccgtccggtggtgctcaagggcctggtgcattccggtgatgccgaagcgcaggcaatggcgatggccgaacgccagttcctggccgaggtggtgcacccgtcgatcgtgcagatcttcaactttgtcgagcacaccgacaggcacggggatccggtcggctacatcgtgatggaatacgtcggcgggcaatcgctcaaacgcagcaagggtcagaaactgcccgtcgcggaggccatcgcctacctgctggagatcctgccggcgctgagctacctgcattccatcggcttggtctacaacgacctgaagccggaaaacatcatgctgaccgaggaacagctcaagctgatcgacctgggcgcggtatcgcggatcaactcgttcggctacctctacgggaccccaggcttccaggcgcccgagatcgtgcggaccggtccgacggtggccaccgacatctacaccgtgggacgcacgctcgcggcgctcacgctggacctgcccacccgcaatggccgttatgtggatgggctacccgaagacgacccggtgctgaaaacctacgactcttacggccggttgctgcgcagggccatcgaccccgatccgcggcaacggttcaccaccgccgaagagatgtccgcgcaattgacgggcgtgttgcgggaggtggtcgcccaggacaccggggtgccgcggccagggctatcaacgatcttcagtcccagtcggtcgacatttggagtggacctgctggtggcgcacaccgacgtgtatctggacgggcaggtgcacgcggagaagctgaccgccaacgagatcgtgaccgcgctgtcggtgccgctggtcgatccgaccgacgtcgcagcttcggtcctgcaggccacggtgctctcccagccggtgcagaccctagactcgctgcgcgcggcccgccacggtgcgctggacgccgacggcgtcgacttctccgagtcagtggagctgccgctaatggaagtccgcgcgctgctggatctcggcgatgtggccaaggccacccgaaaactcgacgatctggccgaacgcgttggctggcgatggcgattggtctggtaccgggccgtcgccgagctgctcaccggcgactatgactcggccaccaaacatttcaccgaggtgctggatacctttcccggcgagctggcgcccaagctcgccctggccgccaccgccgaactagccggcaacaccgacgaacacaagttctatcagacggtgtggagcaccaacgacggcgtgatctcggcggctttcggactggccagagcccggtcggccgaaggtgatcgggtcggcgccgtgcgcacgctcgacgaggtaccgcccacttctcggcatttcaccacggcacggctgaccagcgcggtgactctgttgtccggccggtcaacgagtgaagtcaccgaggaacagatccgcgacgccgcccgaagagtggaggcgctgcccccgaccgaaccacgcgtgctgcagatccgcgccctggtgctgggtggcgcgctggactggctgaaggacaacaaggccagcaccaaccacatcctcggtttcccgttcaccagtcacgggctgcggctgggtgtcgaggcgtcactgcgcagcctggcccgggtagctcccactcaacggcatcgctacacgctggtggacatggccaacaaggtccggcccaccagcacgttctaa

SEQ ID NO: 7 - protein, 140 aa, Mycobacterium tuberculosis
MGSSHHHHHHSSGLVPRGSHMADSTEDFPIPRRMIATTCDAEQYLAAVRDTSPVYYQRYMIDFNNHANLQQATINKAHWFFSLSPAERRDYSEHFYNGDPLTFAWVNHMKIFFNNKGVVAKGTEVCNGYPAGDMSVWNWA

SEQ ID NO: 8 - DNA, 363 bp, Mycobacterium tuberculosis
atggccgactccacggaagactttccaatacctcgccggatgatcgcaaccacctgcgacgccgaacaatatctggcggcggtgcgggataccagtccggtgtactaccagcggtacatgatcgacttcaacaaccatgcaaaccttcagcaagcgacgatcaacaaggcgcactggttcttctcgctgtcaccggcggagcgccgagactactccgaacacttttacaatggcgatccgctgacgtttgcctgggtcaatcacatgaaaatcttcttcaacaacaagggcgtcgtcgctaaagggaccgaggtgtgcaatggatacccagccggcgacatgtcggtgtggaactgggcctaa

SEQ ID NO: 9 - protein, 231 aa, Mycobacterium tuberculosis
MSDPVSYTRKDSIAVISMDDGKVNALGPAMQQALNAAIDNADRDDVGALVITGNGRVFSGGFDLKILTSGEVQPAIDMLRGGFELAYRLLSYPKPVVMACTGHAIAMGAFLLSCGDHRVAAHAYNIQANEVAIGMTIPYAALEIMKLRLTRSAYQQATGLAKTFFGETALAAGFIDEIALPEVVVSRAEEAAREFAGLNQHAHAATKLRSRADALTAIRAGIDGIAAEFGL

SEQ ID NO: 10 - DNA, 696 bp, Mycobacterium tuberculosis
atgagcgacccggtcagctatacccgcaaggattccatcgccgtcatcagtatggacgacggcaaggtcaacgcactgggcccggcgatgcaacaagccctcaatgcagcgatcgacaacgcggaccgtgatgatgttggggcgctggtgatcaccggtaatggccgggtattcagcggaggcttcgacctgaagatcctcacctccggtgaagtgcagcccgcgatcgacatgctcaggggcggcttcgagctggcgtatcgcctcttgtcctaccccaaaccggtggtgatggcgtgcaccggtcacgccatcgccatgggcgcgtttctgttgtcctgcggcgatcatcgggtggcggcccacgcatacaacatccaggccaatgaggtcgcgatcggcatgaccattccgtacgcggcgttagagatcatgaagctgcgactgacccggtcggcataccagcaggcaaccgggctggccaagacgttcttcggggaaaccgcgctggccgccgggtttatcgacgagatcgccctgccggaggtggtggtcagccgcgccgaggaagccgcacgagagttcgccggtctcaaccaacacgcccatgccgcgaccaagttgcgctcccgcgccgacgcgctcactgcgattcgggccgggatcgacgggatagcagccgagttcgggctgtaa

SEQ ID NO: 11 - protein, 359 aa, Mycobacterium tuberculosis
MRYSDSYHTTGRWQPRASTEGFPMGVSIEVNGLTKSFGSSRIWEDVTLTIPAGEVSVLLGPSGTGKSVFLKSLIGLLRPERGSIIIDGTDIIECSAKELYEIRTLFGVLFQDGALFGSMNLYDNTAFPLREHTKKKESEIRDIVMEKLALVGLGGDEKKFPGEISGGMRKRAGLARALVLDPQIILCDEPDSGLDPVRTAYLSQLIMDINAQIDATILIVTHNINIARTVPDNMGMLFRKHLVMFGPREVLLTSDEPVVRQFLNGRRIGPIGMSEEKDEATMAEEQALLDAGHHAGGVEEIEGVPPQISATPGMPERKAVARRQARVREMLHTLPKKAQAAILDDLEGTHKYAVHEIGQ

SEQ ID NO: 12 - DNA, 1080 bp, Mycobacterium tuberculosis
atgcgatacagtgactcataccacacaacgggccggtggcagccacgagcgtcgacagaagggtttcccatgggcgtcagcatcgaggtcaacggactaacgaagtccttcgggtcctcgaggatctgggaagatgtcacgctaacgatccccgccggggaggtcagcgtgctgctgggcccatcgggtaccggcaaatcggtgtttctgaaatctctgatcggcctcctgcggccggagcgcggctcgatcatcatcgacggcaccgacatcatcgaatgctcggccaaggagctttacgagatccgcacattgttcggcgtgctgtttcaggacggtgccctgttcgggtcgatgaacctctacgacaacaccgcgttccccctgcgtgagcacaccaagaaaaaggaaagcgagatccgtgacatcgtcatggagaagctggccctagtcggcctgggtggggacgagaagaagttccccggcgagatctccggcgggatgcgtaagcgtgccggcctagcgcgtgccctggtccttgacccgcagatcattctctgcgacgagcccgactcgggtctggacccggttcgtaccgcctacctgagccagctgatcatggacatcaacgcccagatcgacgccaccatcctgatcgtgacgcacaacatcaacatcgcccgcaccgtgccggacaacatgggcatgttgttccgcaagcatttggtgatgttcgggccgcgggaggtgctactcaccagcgacgagccggtggtgcggcagttcctcaacggccggcgcatcggcccgatcggcatgtccgaggagaaggacgaggccaccatggccgaagagcaggccctgctcgatgccggccaccacgcgggcggtgtcgaggaaatcgagggcgtgccgccgcagatcagcgcgacaccgggcatgccggagcgcaaagcggtcgcccggcgtcaggctcgggttcgcgagatgttgcacacgctgcccaaaaaggcccaggcggcgatcctcgacgatctcgagggcacgcacaagtacgcggtgcacgaaatcggccagtaa

SEQ ID NO: 13 - protein, 271 aa, Mycobacterium tuberculosis
MLPETNQDEVQPNAPVALVTVEIRHPTTDSLTESANRELKHLLINDLPIERQAQDVSWGMTAPGGAPTPVADRFVRYVNRDNTTAASLKNQAIVVETTAYRSFEAFTDVVMRVVDARAQVSSIVGLERIGLRFVLEIRVPAGVDGRITWSNWIDEQLLGPQRFTPGGLVLTEWQGAAVYRELQPGKSLIVRYGPGMGQALDPNYHLRRITPAQTGPFFLLDIDSFWTPSGGSIPEYNRDALVSTFQDLYGPAQVVFQEMITSRLKDELLRQ

SEQ ID NO: 14 - DNA, 822 bp, Mycobacterium tuberculosis
atgctccccgagacaaatcaggatgaggtccagcccaacgcacccgttgccctggtgacggtggaaatccgtcacccgacaacggattcgctcaccgaatcagcgaaccgggagctcaaacacctgcttatcaatgatctaccgatcgaacgccaggcgcaggacgtcagctgggggatgacggcgcccggtggagcccccaccccggtcgcggatcgtttcgttcgttatgtcaatcgcgataacaccaccgccgcttcactgaagaaccaggcgatagtcgtggagaccaccgcctaccgcagctttgaggcctttaccgacgttgtgatgcgggtcgtggatgctcgcgcgcaggtctcgtcaatcgttgggttggagcgtatcggtcttcgctttgttctggagatccgcgtccccgcgggtgtcgacggccggatcacgtggagcaactggatcgacgagcagctgctcgggccgcagcgtttcactcccggcggcctggtcctgaccgagtggcagggtgccgcagtctaccgtgagctacaaccaggcaaatcgctcatcgtgcgctacggcccgggtatgggccaagcgcttgatcccaattaccatctgcgccgaataacacccgcccaaaccggaccattcttcctgctggacatcgatagcttttggactcccagtggcggctccattcccgagtacaacagggacgccttagtgtcgacattccaggacctgtacggtccggcccaggtcgtgtttcaggagatgatcaccagtcgcctgaaagatgagctgcttcgccagtaaaagctt

SEQ ID NO: 15 - protein, 373 aa, Mycobacterium tuberculosis
MGSSHHHHHHSSGLVPRGSHMGCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 16 - DNA, 1122 bp, Mycobacterium tuberculosis
atgggcagcagccatcatcatcatcatcacagcagcggcctggtgccgcgcggcagccatatgggctgtggctcgaaaccaccgagcggttcgcctgaaacgggcgccggcgccggtactgtcgcgactacccccgcgtcgtcgccggtgacgttggcggagaccggtagcacgctgctctacccgctgttcaacctgtggggtccggcctttcacgagaggtatccgaacgtcacgatcaccgctcagggcaccggttctggtgccgggatcgcgcaggccgccgccgggacggtcaacattggggcctccgacgcctatctgtcggaaggtgatatggccgcgcacaaggggctgatgaacatcgcgctagccatctccgctcagcaggtcaactacaacctgcccggagtgagcgagcacctcaagctgaacggaaaagtcctggcggccatgtaccagggcaccatcaaaacctgggacgacccgcagatcgctgcgctcaaccccggcgtgaacctgcccggcaccgcggtagttccgctgcaccgctccgacgggtccggtgacaccttcttgttcacccagtacctgtccaagcaagatcccgagggctggggcaagtcgcccggcttcggcaccaccgtcgacttcccggcggtgccgggtgcgctgggtgagaacggcaacggcggcatggtgaccggttgcgccgagacaccgggctgcgtggcctatatcggcatcagcttcctcgaccaggccagtcaacggggactcggcgaggcccaactaggcaatagctctggcaatttcttgttgcccgacgcgcaaagcattcaggccgcggcggctggcttcgcatcgaaaaccccggcgaaccaggcgatttcgatgatcgacgggcccgccccggacggctacccgatcatcaactacgagtacgccatcgtcaacaaccggcaaaaggacgccgccaccgcgcagaccttgcaggcatttctgcactgggcgatcaccgacggcaacaaggcctcgttcctcgaccaggttcatttccagccgctgccgcccgcggtggtgaagttgtctgacgcgttgatcgcgacgatttccagctag

SEQ ID NO: 17 - protein, 323 aa, Mycobacterium tuberculosis
MGSSHHHHHHSSGLVPRGSHMTHMSIFLSRDNKVIVQGITGSEATVHTARMLRAGTQIVGGVNARKAGTTVTHEDKGGRLIKLPVFGSVAEAMEKTGADVSIIFVPPTFAKDAIIEAIDAEIPLLVVITEGIPVQDTAYAWAYNLEAGHKTRIIGPNCPGIISPGQSLAGITPANITGPGPIGLVSKSGTLTYQMMFELRDLGFSTAIGIGGDPVIGTTHIDAIEAFERDPDTKLIVMIGEIGGDAEERAADFIKTNVSKPVVGYVAGFTAPEGKTMGHAGAIVSGSSGTAAAKQEALEAAGVKVGKTPSATAALAREILLSL

SEQ ID NO: 18 - DNA, 978 bp, Mycobacterium tuberculosis
atgggcagcagccatcatcatcatcatcacagcagcggcctggtgccgcgcggcagccatatgactcacatgtccatatttctgagcagggacaacaaggtcattgtgcagggcatcaccggcagtgaggccaccgtccataccgcgcgaatgctgcgggcgggcacgcaaatcgtcggcggtgtgaacgcacgcaaagcgggcaccaccgtcacgcatgaggataagggcggccggctgatcaagctgccggtgttcggcagtgtcgcggaggcgatggaaaagaccggcgccgatgtgtcgatcatcttcgtgccgccgacgttcgccaaggacgccatcatcgaggccatcgacgccgaaattccgctgttggttgtgatcaccgagggaattccggtgcaggacaccgcctatgcctgggcctacaacctcgaggctggccacaagacccgcatcattggccccaactgtcctggcattatcagtcccggtcagtcgctggccggtatcacgccggccaacatcaccggacccggtccaattggtctggtgtccaagtcggggacgttgacctaccagatgatgttcgaactgcgcgaccttggattctccacggcgatcggcatcggtggtgatccggtgattggcactacccacatcgacgccatcgaggccttcgagagggatccggacaccaagctcatcgtgatgatcggcgagatcggtggtgacgccgaggagcgggccgcagacttcatcaagaccaacgtgtccaagccggtcgtcggctatgtcgccggatttaccgcacccgaaggcaagacgatgggccacgccggcgccatcgtctccggctcgtctggcacagcggcggccaagcaagaggccctggaggccgccggtgtgaaggtcggcaagaccccatcggcgaccgcggcgctggcccgggagatcttgctcagtctctagaagctt

SEQ ID NO: 19 - protein, 347 aa, Mycobacterium tuberculosis
MHHHHHHACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 20 - DNA, 1050 bp, Mycobacterium tuberculosis
atgcatcaccatcaccatcacgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgctgagaattc

SEQ ID NO: 21 - protein, 404 aa, Mycobacterium tuberculosis
MGSSHHHHHHSSGLVPRGSHMELVRVTEAGAMAAGRWVGRGDKEGGDGAAVDAMRELVNSVSMRGVVVIGEGEKDHAPMLYNGEEVGNGDGPECDFAVDPIDGTTLMSKGMTNAISVLAVADRGTMFDPSAVFYMNKIAVGPDAAHVLDITAPISENIRAVAKVKDLSVRDMTVCILDRPRHAQLIHDVRATGARIRLITDGDVAGAISACRPHSGTDLLAGIGGTPEGIIAAAAIRCMGGAIQAQLAPRDDAERRKALEAGYDLNQVLTTEDLVSGENVFFCATGVTDGDLLKGVRYYPGGCTTHSIVMRSKSGTVRMIEAYHRLSKLNEYSAIDFTGDSSAVYPLPAAALEHHHHHHAANKARKEAELAAATAEQGASKRVLRGFLLKGGTISGLANGTRPV

SEQ ID NO: 22 - DNA, 1249 bp, Mycobacterium tuberculosis
atgggcagcagccatcatcatcatcatcacagcagcggcctggtgccgcgcggcagccatatggagctggtccgggtgaccgaggccggagccatggccgcgggccgctgggtaggccgcggcgacaaggagggcggcgacggcgcggcggtcgacgcgatgcgcgaactggtcaactcggtttccatgcgcggggtggtggtcatcggcgaaggcgaaaaggaccacgcaccaatgctctacaacggcgaagaagtgggmaacggcgacggaccggaatgcgactttgccgtcgaccccattgacggcaccacgctgatgagcaagggcatgaccaacgccatctcggtgctggcggtagccgatcgcggcaccatgttcgacccgtcggcggtgttctacatgaacaaaatcgccgtcggccccgatgccgcacacgtgctggatatcaccgcgccgatctcggaaaacatccgagcggtcgccaaggtcaaggacctgtcggtgcgagacatgacggtgtgcatcctggacaggccgcggcacgcgcaactcatccacgacgtccgcgccaccggggcccggatccggctgatcaccgatggcgacgtcgccggcgcgatctcggcgtgccgaccgcactccggcaccgacctgctagctgggatcggcggcaccccggagggaatcatcgccgccgcggcgatccgctgcatgggcggggcgatccaggcgcagctcgccccgcgcgacgacgcggaacgccgcaaggccctagaagccggttacgacctgaaccaggtcttgaccaccgaagatctggtgtccggggaaaacgtcttcttctgcgccactggggtcaccgacggcgacctgctcaagggagtgcgttactaccccggcggctgcaccacccattcgatcgtgatgcgctcgaagtccggcaccgtccggatgatcgaggcctaccaccggctttcaaagctcaacgaatactccgcgatcgacttcaccggcgacagcagcgccgtgtacccattgccctaaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtag

SEQ ID NO: 23 - protein, 349 aa, Mycobacterium tuberculosis
MGSSHHHHHHSSGLVPRGSHMSASPLKVAVTGAAGQIGYSLLFRLASGSLLGPDRPIELRLLEIEPALQALEGVVMELDDCAFPLLSGVEIGSDPQKIFDGVSLALLVGARPRGAGMERSDLLEANGAIFTAQGKALNAVAADDVRVGVTGNPANTNALIAMTNAPDIPRERFSALTRLDHNRAISQLAAKTGAAVTDIKKMTIWGNHSATQYPDLFHAEVAGKNAAEVVNDQAWIEDEFIPTVAKRGAAIIDARGASSAASAASATIDAARDWLLGTPADDWVSMAVVSDGSYGVPEGLISSFPVTTKGGNWTIVSGLEIDEFSRGRIDKSTAELADERSAVTELGLI

SEQ ID NO: 24 - DNA, 1056 bp, Mycobacterium tuberculosis
atgggcagcagccatcatcatcatcatcacagcagcggcctggtgccgcgcggcagccatatgagcgctagtcctctcaaggtcgccgttaccggcgccgccggccaaatcggctacagcctgttgttccgcctggccagcggctctttgctgggccctgaccgtccgatcgagctgcggctgctcgagatcgagccggcactgcaggcgctcgagggtgtggtgatggaactcgacgactgcgctttcccgctgttgtccggggtggagatcggttcagatccccagaagatcttcgatggcgtgagcctggccctgctggtcggagcccgcccccggggcgcgggcatggagcgaagtgacctgctggaggccaacggcgcgatcttcaccgctcagggcaaagccctcaacgctgtcgccgcggatgacgttcgcgtcggggtgaccggcaaccccgccaacaccaacgcgctgatcgcgatgaccaatgcgcccgacattccccgcgagcggttctcggcgctcacccggctggaccacaatcgggcgatctcgcagctggccgccaagaccggcgcggcggtcaccgacatcaagaagatgacgatctggggcaatcactcggccacccagtaccccgacctgttccacgcggaggtcgccggaaagaacgcggccgaagtggtcaacgaccaggcctggatcgaggatgaattcatcccgacggtcgccaagcgcggtgcggcgatcatcgatgcgcgcggcgcgtcgtcggccgcctcggccgcgtcggcaaccatcgacgctgcccgggactggttgctggggacgccggcggacgattgggtctcgatggccgtcgtctccgacgggtcctacggggtgccggagggcttgatctcctcgtttccggtcaccaccaagggcggcaactggacgatcgtgagcggcttggagatcgacgagttctcccgcggccggatcgacaagtcaaccgccgagttggctgacgagcgcagcgcggtcaccgagctcggcctgatctgaaagctt

SEQ ID NO: 25 - protein, 456 aa, Mycobacterium tuberculosis
MVSTHAVVAGETLSALALRFYGDAELYRLIAAASGIADPDVVNVGQRLIMPDFTRYTVVAGDTLSALALRFYGDAELNWLIAAASGIADPDVVNVGQRLIMPDFTRYTVVAGDTLSALAARFYGDASLYPLIAAVNGIADPGVIDVGQVLVIFIGRSDGFGLRIVDRNENDPRLWYYRFQTSAIGWNPGVNVLLPDDYRTSGRTYPVLYLFHGGGTDQDFRTFDFLGIRDLTAGKPIIIVMPDGGHAGWYSNPVSSFVGPRNWETFHIAQLLPWIEANFRTYAEYDGRAVAGFSMGGFGALKYAAKYYGHFASASSHSGPASLRRDFGLVVHWANLSSAVLDLGGGTVYGAPLWDQARVSADNPVERIDSYRNKRIFLVAGTSPDPANWFDSVNETQVLAGQREFRERLSNAGIPHESHEVPGGHVFRPDMFRLDLDGIVARLRPASIGAAAERAD

SEQ ID NO: 26 - DNA, 1371 bp, Mycobacterium tuberculosis
atggtcagcacacatgcggttgtcgcgggggagacgctgtcggcgttggcgttgcgcttctatggcgacgcggaactgtatcggctgatcgccgccgccagcgggatcgccgatcccgacgtcgtcaatgtggggcagcggctgattatgcctgacttcacgcgatacaccgttgttgccggggacacgctgtcggcgttggcgttgcgcttctatggcgacgcggaattgaattggctgatcgccgccgccagcgggatcgccgatcccgacgtcgtcaatgtggggcagcggctgattatgcctgacttcacgcgatacaccgttgttgccggggacacgctgtcggcattggctgcgcgcttctatggcgacgcctccctatatccgcttatcgccgccgtcaatggcatcgccgatcctggcgtcatcgacgtcgggcaggtactggtcatattcatcgggcgtagcgacgggttcggcctaaggatcgtggaccgcaacgagaacgatccccgcctgtggtactaccggttccagacctccgcgatcggctggaaccccggagtcaacgtcctgcttcccgatgactaccgcaccagcggacgcacctatcccgtcctctacctgttccacggcggcggcaccgaccaggatttccgcacgttcgactttctgggcatccgcgacctgaccgccggaaagccgatcatcatcgtgatgcccgacggcgggcacgcgggctggtattccaacccggtcagctcgttcgtcggcccacggaactgggagacattccacatcgcccagctgctcccctggatcgaggcgaacttccgaacctacgccgaatacgacggccgcgcggtcgccgggttttcgatgggtggcttcggcgcgctgaagtacgcagcaaagtactacggccacttcgcgtcggcgagcagccactccggaccggcaagtctgcgccgcgacttcggcctggtagtgcattgggcaaacctgtcctcggcggtgctggatctaggcggcggcacggtttacggcgcgccgctctgggaccaagctagggtcagcgccgacaacccggtcgagcgtatcgacagctaccgcaacaagcggatcttcctggtcgccggcaccagtccggacccggccaactggttcgacagcgtgaacgagacccaggtgctagccgggcagagggagttccgcgaacgcctcagcaacgccggcatcccgcatgaatcgcacgaggtgcctggcggtcacgtcttccggcccgacatgttccgtctcgacctcgacggcatcgtcgcccggctgcgccccgcgagcatcggggcggccgcagaacgcgccgattag

SEQ ID NO: 27 - protein, 329 aa, Mycobacterium tuberculosis
HMELVRVTEAGAMAAGRWVGRGDKEGGDGAAVDAMRELVNSVSMRGVVVIGEGEKDHAPMLYNGEEVGNGDGPECDFAVDPIDGTTLMSKGMTNAISVLAVADRGTMFDPSAVFYMNKIAVGPDAAHVLDITAPISENIRAVAKVKDLSVRDMTVCILDRPRHAQLIHDVRATGARIRLITDGDVAGAISACRPHSGTDLLAGIGGTPEGIIAAAAIRCMGGAIQAQLAPRDDAERRKALEAGYDLNQVLTTEDLVSGENVFFCATGVTDGDLLKGVRYYPGGCTTHSIVMRSKSGTVRMIEAYHRLSKLNEYSAIDFTGDSSAVYPLP

SEQ ID NO: 28 - DNA, 996 bp, Mycobacterium tuberculosis
catatggagctggtccgggtgaccgaggccggagccatggccgcgggccgctgggtaggccgcggcgacaaggagggcggcgacggcgcggcggtcgacgcgatgcgcgaactggtcaactcggtttccatgcgcggggtggtggtcatcggcgaaggcgaaaaggaccacgcaccaatgctctacaacggcgaagaagtgggcaacggcgacggaccggaatgcgactttgccgtcgaccccattgacggcaccacgctgatgagcaagggcatgaccaacgccatctcggtgctggcggtagccgatcgcggcaccatgttcgacccgtcggcggtgttctacatgaacaaaatcgccgtcggccccgatgccgcacacgtgctggatatcaccgcgccgatctcggaaaacatccgagcggtcgccaaggtcaaggacctgtcggtgcgagacatgacggtgtgcatcctggacaggccgcggcacgcgcaactcatccacgacgtccgcgccaccggggcccggatccggctgatcaccgatggcgacgtcgccggcgcgatctcggcgtgccgaccgcactccggcaccgacctgctagctgggatcggcggcaccccggagggaatcatcgccgccgcggcgatccgctgcatgggcggggcgatccaggcgcagctcgccccgcgcgacgacgcggaacgccgcaaggccctagaagccggttacgacctgaaccaggtcttgaccaccgaagatctggtgtccggggaaaacgtcttcttctgcgccactggggtcaccgacggcgacctgctcaagggagtgcgttactaccccggcggctgcaccacccattcgatcgtgatgcgctcgaagtccggcaccgtccggatgatcgaggcctaccaccggctttcaaagctcaacgaatactccgcgatcgacttcaccggcgacagcagcgccgtgtacccattgccctaaaagctt

SEQ ID NO: 29 - protein, 236 aa, Mycobacterium tuberculosis
MRTPRRHCRRIAVLAAVSIAATVVAGCSSGSKPSGGPLPDAKPLVEEATAQTKALKSAHMVLTVNGKIPGLSLKTLSGDLTTNPTAATGNVKLTLGGSDIDADFVVFDGILYATLTPNQWSDFGPAADIYDPAQVLNPDTGLANVLANFADAKAEGRDTINGQNTIRISGKVSAQAVNQIAPPFNATQPVPATVWIQETGDHQLAQAQLDRGSGNSVQMTLSKWGEKVQVTKPPVS

SEQ ID NO: 30 - DNA, 710 bp, Mycobacterium tuberculosis
atgcggacccccagacgccactgccgtcgcatcgccgtcctcgccgccgttagcatcgccgccactgtcgttgccggctgctcgtcgggctcgaagccaagcggcggaccacttccggacgcgaagccgctggtcgaggaggccaccgcgcagaccaaggctctcaagagcgcgcacatggtgctgacggtcaacggcaagatcccgggactgtctctgaagacgctgagcggcgatctcaccaccaaccccaccgccgcgacgggaaacgtcaagctcacgctgggtgggtctgatatcgatgccgacttcgtggtgttcgacgggatcctgtacgccaccctgacgcccaaccagtggagcgatttcggtcccgccgccgacatctacgaccccgcccaggtgctgaatccggataccggcctggccaacgtgctggcgaatttcgccgacgcaaaagccgaagggcgggataccatcaacggccagaacaccatccgcatcagcgggaaggtatcggcacaggcggtgaaccagatagcgccgccgttcaacgcgacgcagccggtgccggcgaccgtctggattcaggagaccggcgatcatcaactggcacaggcccagttggaccgcggctcgggcaattccgtccagatgaccttgtcgaaatggggcgagaaggtccaggtcacgaagcccccggtgagctg

SEQ ID NO: 31 - protein, 406 aa, Mycobacterium tuberculosis
MGSSHHHHHHSSGLVPRGSHMKAATQARIDDSPLAWLDAVQRQRHEAGLRRCLRPRPAVATELDLASNDYLGLSRHPAVIDGGVQALRIWGAGATGSRLVTGDTKLHQQFEAELAEFVGAAAGLLFSSGYTANLGAVVGLSGPGSLLVSDARSHASLVDACRLSRARVVVTPHRDVDAVDAALRSRDEQRAVVVTDSVFSADGSLAPVRELLEVCRRHGALLLVDEAHGLGVRGGGRGLLYELGLAGAPDVVMTTTLSKALGSQGGVVLGPTPVRAHLIDAARPFIFDTGLAPAAVGAARAALRVLQAEPWRPQAVLNHAGELARMCGVAAVPDSAMVSVILGEPESAVAAAAACLDAGVKVGCFRPPTVPAGTSRLRLTARASLNAGELELARRVLTDVLAVARR

SEQ ID NO: 32 - DNA, 1227 bp, Mycobacterium tuberculosis
atgggcagcagccatcatcatcatcatcacagcagcggcctggtgccgcgcggcagccatatgaaagccgccacgcaggcacggatcgacgattcaccgttggcctggttggacgcggtgcagcggcagcgccacgaggccggactgcggcgctgcctgcggccgcgtcccgcggtcgccaccgagctggacttggcctccaacgactatctcggtctgtcccgacatcccgccgtcatcgacggcggcgtccaggcgctgcggatctggggcgccggcgccaccgggtcgcgcctggttaccggcgacaccaagctgcaccagcaattcgaggccgagctcgccgagttygtcggcgctgccgcgggattgctgttctcctctggctacacggccaacctgggcgccgtggtcggcctgtccggcccgggttccctgctggtgtccgacgcccgttcgcatgcgtcgttggtggatgcctgtcggctgtcgcgggcgcgggttgtggtgacgccgcaccgcgacgtcgacgccgtggacgccgcgctgcgatcgcgcgacgagcagcgcgccgtcgtcgtcaccgactcggtgttcagcgccgacggctcgctggcgccggttcgggagttgcttgaggtctgccggcgtcatggtgcgctgcttctggtggacgaggcgcacggcctgggtgtgcgtggcggcggacgcgggctgctctacgagttaggtctagcgggtgcgcccgacgtggtgatgaccaccacgctgtccaaggcgctgggcagccagggtggtgtggtgctcgggccgacgccggtgcgggcccatctgatcgatgctgcccggccgttcatcttcgacaccggtctggcgccggcggcggtgggtgccgcacgggccgcgctgcgcgtcttgcaggccgagccgtggcgaccgcaggcggtgctcaaccacgctggtgaacttgcgcggatgtgcggtgtggctgcggtgccggactcggcgatggtgtcggtgatcctgggcgagccggagtcggcagtggccgccgcggcggcctgcctggacgccggggtcaaggtgggctgcttccggccgccgacggtgcccgcgggtacgtcgcggctgcggctgaccgcgcgcgcatcgctgaacgccggcgagctcgagctggcccggcgggtgctgacggatgttctcgccgtggcgcgccgttgaaagctt

SEQ ID NO: 33 - protein, 393 aa, Mycobacterium tuberculosis
MDFGALPPEVNSVRMYAGPGSAPMVAAASAWNGLAAELSSAATGYETVITQLSSEGWLGPASAAMAEAVAPYVAWMSAAAAQAEQAATQARAAAAAFEAAFAATVPPPLIAANRASLMQLISTNVFGQNTSAIAAAEAQYGEMWAQDSAAMYAYAGSSASASAVTPFSTPPQIANPTAQGTQAAAVATAAGTAQSTLTEMITGLPNALQSLTSPLLQSSNGPLSWLWQILFGTPNFPTSISALLTDLQPYASFFYNTEGLPYFSIGMGNNFIQSAKTLGLIGSAAPAAVAAAGDAAKGLPGLGGMLGGGPVAAGLGNAASVGKLSVPPVWSGPLPGSVTPGAAPLPVSTVSAAPEAAPGSLLGGLPLAGAGGAGAGPRYGFRPTVMARPPFAG

SEQ ID NO: 34 - DNA, 1188 bp, Mycobacterium tuberculosis
atggattttggggcgttgccgccggaggtcaattcggtgcggatgtatgccggtcctggctcggcaccaatggtcgctgcggcgtcggcctggaacgggttggccgcggagctgagttcggcggccaccggttatgagacggtgatcactcagctcagcagtgaggggtggctaggtccggcgtcagcggcgatggccgaggcagttgcgccgtatgtggcgtggatgagtgccgctgcggcgcaagccgagcaggcggccacacaggccagggccgccgcggccgcttttgaggcggcgtttgccgcgacggtgcctccgccgttgatcgcggccaaccgggcttcgttgatgcagctgatctcgacgaatgtctttggtcagaacacctcggcgatcgcggccgccgaagctcagtacggcgagatgtgggcccaagactccgcggcgatgtatgcctacgcgggcagttcggcgagcgcctcggcggtcacgccgtttagcacgccgccgcagattgccaacccgaccgctcagggtacgcaggccgcggccgtggccaccgccgccggtaccgcccagtcgacgctgacggagatgatcaccgggctacccaacgcgctgcaaagcctcacctcacctctgttgcagtcgtctaacggtccgctgtcgtggctgtggcagatcttgttcggcacgcccaatttccccacctcaatttcggcactgctgaccgacctgcagccctacgcgagcttcttctataacaccgagggcctgccgtacttcagcatcggcatgggcaacaacttcattcagtcggccaagaccctgggattgatcggctcggcggcaccggctgcggtcgcggctgctggggatgccgccaagggcttgcctggactgggcgggatgctcggtggcgggccggtggcggcgggtctgggcaatgcggcttcggttggcaagctgtcggtgccgccggtgtggagtggaccgttgcccgggtcggtgactccgggggctgctccgctaccggtgagtacggtcagtgccgccccggaggcggcgcccggaagcctgttgggcggcctgccgctagctggtgcgggcggggccggcgcgggtccacgctacggattccgtcccaccgtcatggctcgcccacccttcgccggatagaagctt

SEQ ID NO: 35 - protein, 143 aa, Mycobacterium tuberculosis
MITNLRRRTAMAAAGLGAALGLGILLVPTVDAHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACN

SEQ ID NO: 36 - DNA, 432 bp, Mycobacterium tuberculosis
atgatcacaaacctccgacgccgaaccgcgatggcagccgccggcctaggggctgctctcgggctgggcatcctgctggttccgacggtggacgcccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaactaa

SEQ ID NO: 37 - protein, 163 aa, Mycobacterium tuberculosis
MVTDMNPDIEKDQTSDEVTVETTSVFRADFLSELDAPAQAGTESAVSGVEGLPPGSALLVVKRGPNAGSRFLLDQAITSAGRHPDSDIFLDDVTVSRRHAEFRLENNEFNVVDVGSLNGTYVNREPVDSAVLANGDEVQIGKFRLVFLTGPKQGEDDGSTGGP

SEQ ID NO: 38 - DNA, 489 bp, Mycobacterium tuberculosis
gtgacggacatgaacccggatattgagaaggaccagacctccgatgaagtcacggtagagacgacctccgtcttccgcgcagacttcctcagcgagctggacgctcctgcgcaagcgggtacggagagcgcggtctccggggtggaagggctcccgccgggctcggcgttgctggtagtcaaacgaggccccaacgccgggtcccggttcctactcgaccaagccatcacgtcggctggtcggcatcccgacagcgacatatttctcgacgacgtgaccgtgagccgtcgccatgctgaattccggttggaaaacaacgaattcaatgtcgtcgatgtcgggagtctcaacggcacctacgtcaaccgcgagcccgtggattcggcggtgctggcgaacggcgacgaggtccagatcggcaagttccggttggtgttcttgaccggacccaagcaaggcgaggatgacgggagtaccgggggcccgtga

SEQ ID NO: 39 - protein, 741 aa, Mycobacterium tuberculosis
MTDRVSVGNLRIARVLYDFVNNEALPGTDIDPDSFWAGVDKVVADLTPQNQALLNARDELQAQIDKWHRRRVIEPIDMDAYRQFLTEIGYLLPEPDDFTITTSGVDAEITTTAGPQLVVPVLNARFALNAANARWGSLYDALYGTDVIPETDGAEKGPTYNKVRGDKVIAYARKFLDDSVPLSSGSFGDATGFTVQDGQLVVALPDKSTGLANPGQFAGYTGAAESPTSVLLINHGLHIEILIDPESQVGTTDRAGVKDVILESAITTIMDFEDSVAAVDAADKVLGYRNWLGLNKGDLAAAVDKDGTAFLRVLNRDRNYTAPGGGQFTLPGRSLMFVRNVGHLMTNDAIVDTDGSEVFEGIMDALFTGLIAIHGLKASDVNGPLINSRTGSIYIVKPKMHGPAEVAFTCELFSRVEDVLGLPQNTMKIGIMDEERRTTVNLKACIKAAADRVVFINTGFLDRTGDEIHTSMEAGPMVRKGTMKSQPWILAYEDHNVDAGLAAGFSGRAQVGKGMWTMTELMADMVETKIAQPRAGASTAWVPSPTAATLHALHYHQVDVAAVQQGLAGKRRATIEQLLTIPLAKELAWAPDEIREEVDNNCQSILGYVVRWVDQGVGCSKVPDIHDVALMEDRATLRISSQLLANWLRHGVITSADVRASLERMAPLVDRQNAGDVAYRPMAPNFDDSIAFLAAQELILSGAQQPNGYTEPILHRRRREFKARAAEKPAPSDRAGDDAAR

SEQ ID NO: 40 - DNA, 2226 bp, Mycobacterium tuberculosis
atgacagatcgcgtgtcggtgggcaacttgcgcatcgctcgggtgctctacgacttcgtgaacaatgaagccctgcctggcaccgatatcgacccggacagcttctgggcgggcgtcgacaaggtcgtcgccgacctgaccccgcagaaccaagctctgttgaacgcccgcgacgagctgcaggcgcagatcgacaagtggcaccggcgtcgggtgatcgagcccatcgacatggatgcctaccgccagttcctcaccgagatcggctacctgcttcccgaacctgatgacttcaccatcaccacgtccggtgtcgacgctgagatcaccacgaccgccggcccccagctggtggtgccggtgctcaacgcgcggtttgctctgaacgcggccaacgctcgctggggctccctctacgacgccttgtatggcaccgatgtcatccccgagaccgacggcgccgaaaaaggccccacgtacaacaaggttcgtggcgacaaggtgatcgcgtatgcccgcaagttcctcgacgacagtgttccgctgtcgtcgggttcctttggcgacgccaccggtttcacagtgcaggatggccagctcgtggttgccttgccggataagtccaccggcctggccaaccccggccagttcgccggctacaccggcgcagccgagtcgccgacatcggtgctgctaatcaatcacggtttgcacatcgagatcctgatcgatccggagtcgcaggtcggcaccaccgaccgggccggcgtcaaggacgtgatcctggaatccgcgatcaccacgatcatggacttcgaggactcggtggccgccgtggacgccgccgacaaggtgctgggttatcggaactggctcggcctgaacaagggcgacctggcagcagcggtagacaaggacggcaccgctttcctgcgggtgctcaatagggaccggaactacaccgcacccggcggtggccagttcacgctgcctggacgcagcctcatgttcgtccgcaacgtcggtcacttgatgacgaatgacgccatcgtcgacactgacggcagcgaggtgttcgaaggcatcatggatgccctattcaccggcctgatcgccatccacgggctaaaggccagcgacgtcaacgggccgctgatcaacagccgcaccggctccatctacatcgtcaagccgaagatgcacggtccggccgaggtggcgtttacctgcgaactgttcagccgggttgaagatgtgctggggttgccgcaaaacaccatgaagatcggcatcatggacgaggaacgccggaccacggtcaacctcaaggcgtgcatcaaagctgccgcggaccgcgtggtgttcatcaacaccgggttcctggaccgcaccggcgatgaaatccacacctcgatggaggccggcccgatggtgcgcaagggcaccatgaagagccagccgtggatcttggcctacgaggaccacaacgtcgatgccggcctggccgccgggttcagcggccgagcccaggtcggcaagggcatgtggacaatgaccgagctgatggccgacatggtcgagacaaaaatcgcccagccgcgcgccggggccagcaccgcctgggttccctctcccactgcggccaccctgcatgcgctgcactaccaccaggtcgacgtcgccgcggtgcaacaaggactggcggggaagcgtcgcgccaccatcgaacaattgctgaccattccgctggccaaggaattggcctgggctcccgacgagatccgcgaagaggtcgacaacaactgtcaatccatcctcggctacgtggttcgctgggttgatcaaggtgtcggctgctcgaaggtgcccgacatccacgacgtcgcgctcatggaggaccgggccacgctgcgaatctccagccaattgttggccaactggctgcgccacggtgtgatcaccagcgcggatgtgcgggccagcttggagcggatggcgccgttggtcgatcgacaaaacgcgggcgacgtggcataccgaccgatggcacccaacttcgacgacagtatcgccttcctggccgcgcaggagctgatcttgtccggggcccagcagcccaacggctacaccgagccgatcctgcaccgacgtcgtcgggagtttaaggcccgggccgctgagaagccggccccatcggacagggccggtgacgatgcggcccgctag

SEQ ID NO: 41 - protein, 325 aa, Mycobacterium tuberculosis
MHQVDPNLTRRKGRLAALAIAAMASASLVTVAVPATANADPEPAPPVPTTAASPPSTAAAPPAPATPVAPPPPAAANTPNAQPGDPNAAPPPADPNAPPPPVIAPNAPQPVRIDNPVGGFSFALPAGWVESDAAHFDYGSALLSKTTGDPPFPGQPPPVANDTRIVLGRLDQKLYASAEATDSKAAARLGSDMGEFYMPYPGTRINQETVSLDANGVSGSASYYEVKFSDPSKPNGQIWTGVIGSPAANAPDAGPPQRWFVVWLGTANNPVDKGAAKALAESIRPLVAPPPAPAPAPAEPAPAPAPAGEVAPTPTTPTPQRTLPA

SEQ ID NO: 42 - DNA, 978 bp, Mycobacterium tuberculosis
atgcatcaggtggaccccaacttgacacgtcgcaagggacgattggcggcactggctatcgcggcgatggccagcgccagcctggtgaccgttgcggtgcccgcgaccgccaacgccgatccggagccagcgcccccggtacccacaacggccgcctcgccgccgtcgaccgctgcagcgccacccgcaccggcgacacctgttgcccccccaccaccggccgccgccaacacgccgaatgcccagccgggcgatcccaacgcagcacctccgccggccgacccgaacgcaccgccgccacctgtcattgccccaaacgcaccccaacctgtccggatcgacaacccggttggaggattcagcttcgcgctgcctgctggctgggtggagtctgacgccgcccacttcgactacggttcagcactcctcagcaaaaccaccggggacccgccatttcccggacagccgccgccggtggccaatgacacccgtatcgtgctcggccggctagaccaaaagctttacgccagcgccgaagccaccgactccaaggccgcggcccggttgggctcggacatgggtgagttctatatgccctacccgggcacccggatcaaccaggaaaccgtctcgctcgacgccaacggggtgtctggaagcgcgtcgtattacgaagtcaagttcagcgatccgagtaagccgaacggccagatctggacgggcgtaatcggctcgcccgcggcgaacgcaccggacgccgggccccctcagcgctggtttgtggtatggctcgggaccgccaacaacccggtggacaagggcgcggccaaggcgctggccgaatcgatccggcctttggtcgccccgccgccggcgccggcaccggctcctgcagagcccgctccggcgccggcgccggccggggaagtcgctcctaccccgacgacaccgacaccgcagcggaccttaccggcctga

SEQ ID NO: 43 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 44 - DNA, 978 bp, Mycobacterium tuberculosis
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctga

SEQ ID NO: 45 - protein, 740 aa, Mycobacterium tuberculosis
VPEQHPPITETTTGAASNGCPVVGHMKYPVEGGGNQDWWPNRLNLKVLHQNPAVADPMGAAFDYAAEVATIDVDALTRDIEEVMTTSQPWWPADYGHYGPLFIRMAWHAAGTYRIHDGRGGAGGGMQRFAPLNSWPDNASLDKARRLLWPVKKKYGKKLSWADLIVFAGNCALESMGFKTFGFGFGRVDQWEPDEVYWGKEATWLGDERYSGKRDLENPLAAVQMGLIYVNPEGPNGNPDPMAAAVDIRETFRRMAMNDVETAALIVGGHTFGKTHGAGPADLVGPEPEAAPLEQMGLGWKSSYGTGTGKDAITSGIEVVWTNTPTKWDNSFLEILYGYEWELTKSPAGAWQYTAKDGAGAGTIPDPFGGPGRSPTMLATDLSLRVDPIYERITRRWLEHPEELADEFAKAWYKLIHRDMGPVARYLGPLVPKQTLLWQDPVPAVSHDLVGEAEIASLKSQIRASGLTVSQLVSTAWAAASSFRGSDKRGGANGGRIRLQPQVGWEVNDPDGDLRKVIRTLEEIQESFNSAAPGNIKVSFADLVVLGGCAAIEKAAKAAGHNITVPFTPGRTDASQEQTDVESFAVLEPKADGFRNYLGKGNPLPAEYMLLDKANLLTLSAPEMTVLVGGLRVLGANYKRLPLGVFTEASESLTNDFFVNLLDMGITWEPSPADDGTYQGKDGSGKVKWTGSRVDLVFGSNSELRALVEVYGADDAQPKFVQDFVAAWDKVMNLDRFDVR

SEQ ID NO: 46 - DNA, 2223 bp, Mycobacterium tuberculosis
gtgcccgagcaacacccacccattacagaaaccaccaccggagccgctagcaacggctgtcccgtcgtgggtcatatgaaataccccgtcgagggcggcggaaaccaggactggtggcccaaccggctcaatctgaaggtactgcaccaaaacccggccgtcgctgacccgatgggtgcggcgttcgactatgccgcggaggtcgcgaccatcgacgttgacgccctgacgcgggacatcgaggaagtgatgaccacctcgcagccgtggtggcccgccgactacggccactacgggccgctgtttatccggatggcgtggcacgctgccggcacctaccgcatccacgacggccgcggcggcgccgggggcggcatgcagcggttcgcgccgcttaacagctggcccgacaacgccagcttggacaaggcgcgccggctgctgtggccggtcaagaagaagtacggcaagaagctctcatgggcggacctgattgttttcgccggcaactgcgcgctggaatcgatgggcttcaagacgttcgggttcggcttcggccgggtcgaccagtgggagcccgatgaggtctattggggcaaggaagccacctggctcggcgatgagcgttacagcggtaagcgggatctggagaacccgctggccgcggtgcagatggggctgatctacgtgaacccggaggggccgaacggcaacccggaccccatggccgcggcggtcgacattcgcgagacgtttcggcgcatggccatgaacgacgtcgaaacagcggcgctgatcgtcggcggtcacactttcggtaagacccatggcgccggcccggccgatctggtcggccccgaacccgaggctgctccgctggagcagatgggcttgggctggaagagctcgtatggcaccggaaccggtaaggacgcgatcaccagcggcatcgaggtcgtatggacgaacaccccgacgaaatgggacaacagtttcctcgagatcctgtacggctacgagtgggagctgacgaagagccctgctggcgcttggcaatacaccgccaaggacggcgccggtgccggcaccatcccggacccgttcggcgggccagggcgctccccgacgatgctggccactgacctctcgctgcgggtggatccgatctatgagcggatcacgcgtcgctggctggaacaccccgaggaattggccgacgagttcgccaaggcctggtacaagctgatccaccgagacatgggtcccgttgcgagataccttgggccgctggtccccaagcagaccctgctgtggcaggatccggtccctgcggtcagccacgacctcgtcggcgaagccgagattgccagccttaagagccagatccgggcatcgggattgactgtctcacagctagtttcgaccgcatgggcggcggcgtcgtcgttccgtggtagcgacaagcgcggcggcgccaacggtggtcgcatccgcctgcagccacaagtcgggtgggaggtcaacgaccccgacggggatctgcgcaaggtcattcgcaccctggaagagatccaggagtcattcaactccgcggcgccggggaacatcaaagtgtccttcgccgacctcgtcgtgctcggtggctgtgccgccatagagaaagcagcaaaggcggctggccacaacatcacggtgcccttcaccccgggccgcacggatgcgtcgcaggaacaaaccgacgtggaatcctttgccgtgctggagcccaaggcagatggcttccgaaactacctcggaaagggcaacccgttgccggccgagtacatgctgctcgacaaggcgaacctgcttacgctcagtgcccctgagatgacggtgctggtaggtggcctgcgcgtcctcggcgcaaactacaagcgcttaccgctgggcgtgttcaccgaggcctccgagtcactgaccaacgacttcttcgtgaacctgctcgacatgggtatcacctgggagccctcgccagcagatgacgggacctaccagggcaaggatggcagtggcaaggtgaagtggaccggcagccgcgtggacctggtcttcgggtccaactcggagttgcgggcgcttgtcgaggtctatggcgccgatgacgcgcagccgaagttcgtgcaggacttcgtcgctgcctgggacaaggtgatgaacctcgacaggttcgacgtgcgctga

SEQ ID NO: 47 - protein, 206 aa, Mycobacterium tuberculosis
MAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 48 - DNA, 627 bp, Mycobacterium tuberculosis
atggcgcccaagacctactgcgaggagttgaaaggcaccgataccggccaggcgtgccagattcaaatgtccgacccggcctacaacatcaacatcagcctgcccagttactaccccgaccagaagtcgctggaaaattacatcgcccagacgcgcgacaagttcctcagcgcggccacatcgtccactccacgcgaagccccctacgaattgaatatcacctcggccacataccagtccgcgataccgccgcgtggtacgcaggccgtggtgctcaaggtctaccagaacgccggcggcacgcacccaacgaccacgtacaaggccttcgattgggaccaggcctatcgcaagccaatcacctatgacacgctgtggcaggctgacaccgatccgctgccagtcgtcttccccattgtgcaaggtgaactgagcaagcagaccggacaacaggtatcgatagcgccgaatgccggcttggacccggtgaattatcagaacttcgcagtcacgaacgacggggtgattttcttcttcaacccgggggagttgctgcccgaagcagccggcccaacccaggtattggtcccacgttccgcgatcgactcgatgctggcctagaagctt

SEQ ID NO: 49 - protein, 217 aa, Mycobacterium tuberculosis
MTPRSLVRIVGVVVATTLALVSAPAGGRAAHADPCSDIAVVFARGTHQASGLGDVGEAFVDSLTSQVGGRSIGVYAVNYPASDDYRASASNGSDDASAHIQRTVASCPNTRIVLGGYSQGATVIDLSTSAMPPAVADHVAAVALFGEPSSGFSSMLWGGGSLPTIGPLYSSKTINLCAPDDPICTGGGNIMAHVSYVQSGMTSQAATFAANRLDHAG

SEQ ID NO: 50 - DNA, 654 bp, Mycobacterium tuberculosis
atgactccacgcagccttgttcgcatcgttggtgtcgtggttgcgacgaccttggcgctggtgagcgcacccgccggcggtcgtgccgcgcatgcggatccgtgttcggacatcgcggtcgttttcgctcgcggcacgcatcaggcttctggtcttggcgacgtcggtgaggcgttcgtcgactcgcttacctcgcaagttggcgggcggtcgattggggtctacgcggtgaactacccagcaagcgacgactaccgcgcgagcgcgtcaaacggttccgatgatgcgagcgcccacatccagcgcaccgtcgccagctgcccgaacaccaggattgtgcttggtggctattcgcagggtgcgacggtcatcgatttgtccacctcggcgatgccgcccgcggtggcagatcatgtcgccgctgtcgcccttttcggcgagccatccagtggtttctccagcatgttgtggggcggcgggtcgttgccgacaatcggtccgctgtatagctctaagaccataaacttgtgtgctcccgacgatccaatatgcaccggaggcggcaatattatggcgcatgtttcgtatgttcagtcggggatgacaagccaggcggcgacattcgcggcgaacaggctcgatcacgccggatga

SEQ ID NO: 51 - protein, 144 aa, Mycobacterium tuberculosis
MATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTN

SEQ ID NO: 52 - DNA, 435 bp, Mycobacterium tuberculosis
atggccaccacccttcccgttcagcgccacccgcggtccctcttccccgagttttctgagctgttcgcggccttcccgtcattcgccggactccggcccaccttcgacacccggttgatgcggctggaagacgagatgaaagaggggcgctacgaggtacgcgcggagcttcccggggtcgaccccgacaaggacgtcgacattatggtccgcgatggtcagctgaccatcaaggccgagcgcaccgagcagaaggacttcgacggtcgctcggaattcgcgtacggttccttcgttcgcacggtgtcgctgccggtaggtgctgacgaggacgacattaaggccacctacgacaagggcattcttactgtgtcggtggcggtttcggaagggaagccaaccgaaaagcacattcagatccggtccaccaactga

SEQ ID NO: 53 - protein, 331 aa, Mycobacterium tuberculosis
MPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHR

SEQ ID NO: 54 - DNA, 1002 bp, Mycobacterium tuberculosis
atgccggacaccatggtgaccaccgatgtcatcaagagcgcggtgcagttggcctgccgcgcaccgtcgctccacaacagccagccctggcgctggatagccgaggaccacacggttgcgctgttcctcgacaaggatcgggtgctttacgcgaccgaccactccggccgggaagcgctgctggggtgcggcgccgtactcgaccactttcgggtggcgatggcggccgcgggtaccaccgccaatgtggaacggtttcccaaccccaacgatcctttgcatctggcgtcaattgacttcagcccggccgatttcgtcaccgagggccaccgtctaagggcggatgcgatcctactgcgccgtaccgaccggctgcctttcgccgagccgccggattgggacttggtggagtcgcagttgcgcacgaccgtcaccgccgacacggtgcgcatcgacgtcatcgccgacgatatgcgtcccgaactggcggcggcgtccaaactcaccgaatcgctgcggctctacgattcgtcgtatcatgccgaactcttttggtggacaggggcttttgagacttctgagggcataccgcacagttcattggtatcggcggccgaaagtgaccgggtcaccttcggacgcgacttcccggtcgtcgccaacaccgataggcgcccggagtttggccacgaccgctctaaggtcctggtgctctccacctacgacaacgaacgcgccagcctactgcgctgcggcgagatgctttccgccgtattgcttgacgccaccatggctgggcttgccacctgcacgctgacccacatcaccgaactgcacgccagccgagacctggtcgcagcgctgattgggcagcccgcaactccgcaagccttggttcgcgtcggtctggccccggagatggaagagccgccaccggcaacgcctcggcgaccaatcgatgaagtgtttcacgttcgggctaaggatcaccggtaggaattc

SEQ ID NO: 55 - protein, 478 aa, Mycobacterium tuberculosis
VTEKTPDDVFKLAKDEKVEYVDVRFCDLPGIMQHFTIPASAFDKSVFDDGLAFDGSSIRGFQSIHESDMLLLPDPETARIDPFRAAKTLNINFFVHDPFTLEPYSRDPRNIARKAENYLISTGIADTAYFGAEAEFYIFDSVSFDSRANGSFYEVDAISGWWNTGAATEADGSPNRGYKVRHKGGYFPVAPNDQYVDLRDKMLTNLINSGFILEKGHHEVGSGGQAEINYQFNSLLHAADDMQLYKYIIKNTAWQNGKTVTFMPKPLFGDNGSGMHCHQSLWKDGAPLMYDETGYAGLSDTARHYIGGLLHHAPSLLAFTNPTVNSYKRLVPGYEAPINLVYSQRNRSACVRIPITGSNPKAKRLEFRSPDSSGNPYLAFSAMLMAGLDGIKNKIEPQAPVDKDLYELPPEEAASIPQTPTQLSDVIDRLEADHEYLTEGGVFTNDLIETWISFKRENEIEPVNIRPHPYEFALYYDV

SEQ ID NO: 56 - DNA, 1437 bp, Mycobacterium tuberculosis
gtgacggaaaagacgcccgacgacgtcttcaaacttgccaaggacgagaaggtcgaatatgtcgacgtccggttctgtgacctgcctggcatcatgcagcacttcacgattccggcttcggcctttgacaagagcgtgtttgacgacggcttggcctttgacggctcgtcgattcgcgggttccagtcgatccacgaatccgacatgttgcttcttcccgatcccgagacggcgcgcatcgacccgttccgcgcggccaagacgctgaatatcaacttctttgtgcacgacccgttcaccctggagccgtactcccgcgacccgcgcaacatcgcccgcaaggccgagaactacctgatcagcactggcatcgccgacaccgcatacttcggcgccgaggccgagttctacattttcgattcggtgagcttcgactcgcgcgccaacggctccttctacgaggtggacgccatctcggggtggtggaacaccggcgcggcgaccgaggccgacggcagtcccaaccggggctacaaggtccgccacaagggcgggtatttcccagtggcccccaacgaccaatacgtcgacctgcgcgacaagatgctgaccaacctgatcaactccggcttcatcctggagaagggccaccacgaggtgggcagcggcggacaggccgagatcaactaccagttcaattcgctgctgcacgccgccgacgacatgcagttgtacaagtacatcatcaagaacaccgcctggcagaacggcaaaacggtcacgttcatgcccaagccgctgttcggcgacaacgggtccggcatgcactgtcatcagtcgctgtggaaggacggggccccgctgatgtacgacgagacgggttatgccggtctgtcggacacggcccgtcattacatcggcggcctgttacaccacgcgccgtcgctgctggccttcaccaacccgacggtgaactcctacaagcggctggttcccggttacgaggccccgatcaacctggtctatagccagcgcaaccggtcggcatgcgtgcgcatcccgatcaccggcagcaacccgaaggccaagcggctggagttccgaagccccgactcgtcgggcaacccgtatctggcgttctcggccatgctgatggcaggcctggacggtatcaagaacaagatcgagccgcaggcgcccgtcgacaaggatctctacgagctgccgccggaagaggccgcgagtatcccgcagactccgacccagctgtcagatgtgatcgaccgtctcgaggccgaccacgaatacctcaccgaaggaggggtgttcacaaacgacctgatcgagacgtggatcagtttcaagcgcgaaaacgagatcgagccggtcaacatccggccgcatccctacgaattcgcgctgtactacgacgtttaa

SEQ ID NO: 57 - protein, 145 aa, Mycobacterium tuberculosis
MDDAGLDPNAAAGPDAVGFDPNLPPAPDAAPVDTPPAPEDAGFDPNLPPPLAPDFLSPPAEEAPPVPVAYSVNWDAIAQCESGGNWSINTGNGYYGGLQFTAGTWRANGGSGSAANASREEQIRVAENVLRSQGIRAWPVCGRRG

SEQ ID NO: 58 - DNA, 444 bp, Mycobacterium tuberculosis
atggacgacgcgggcttggacccaaacgccgcagccggcccggatgccgtgggctttgacccgaacctgccgccggccccggacgctgcacccgtcgatactccgccggctccggaggacgcgggctttgatcccaacctccccccgccgctggccccggacttcctgtccccgcctgcggaggaagcgcctcccgtgcccgtggcctacagcgtgaactgggacgcgatcgcgcagtgcgagtccggtggaaactggtcgatcaacaccggtaacggttactacggcggcctgcagttcaccgccggcacctggcgtgccaacggtggctcggggtccgcggccaacgcgagccgggaggagcagatccgggtggctgagaacgtgctgcgttcgcagggtatccgcgcctggccggtctgcggccgccgcggctgagaattc

SEQ ID NO: 59 - protein, 580 aa, Mycobacterium tuberculosis
MNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLF

SEQ ID NO: 60 - DNA, 1743 bp, Mycobacterium tuberculosis
atgaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttctaa

SEQ ID NO: 61 - protein, 297 aa, Mycobacterium tuberculosis
MSSGNSSLGIIVGIDDSPAAQVAVRWAARDAELRKIPLTLVHAVSPEVATWLEVPLPPGVLRWQQDHGRHLIDDALKVVEQASLRAGPPTVHSEIVPAAAVPTLVDMSKDAVLMVVGCLGSGRWPGRLLGSVSSGLLRHAHCPVVIIHDEDSVMPHPQQAPVLVGVDGSSASELATAIAFDEASRRNVDLVALHAWSDVDVSEWPGIDWPATQSMAEQVLAERLAGWQERYPNVAITRVVVRDQPARQLVQRSEEAQLVVVGSRGRGGYAGMLVGSVGETVAQLARTPVIVARESLT

SEQ ID NO: 62 - DNA, 894 bp, Mycobacterium tuberculosis
atgtcatcgggcaattcatctctgggaattatcgtcgggatcgacgattcaccggccgcacaggttgcggtgcggtgggcagctcgggatgcggagttgcgaaaaatccctctgacgctcgtgcacgcggtgtcgccggaagtagccacctggctggaggtgccactgccgccgggcgtgctgcgatggcagcaggatcacgggcgccacctgatcgacgacgcactcaaggtggttgaacaggcttcgctgcgcgctggtccccccacggtccacagtgaaatcgttccggcggcagccgttcccacattggtcgacatgtccaaagacgcagtgctgatggtcgtgggttgtctcggaagtgggcggtggccgggccggctgctcggttcggtcagttccggcctgctccgccacgcgcactgtccggtcgtgatcatccacgacgaagattcggtgatgccgcatccccagcaagcgccggtgctagttggcgttgacggctcgtcggcctccgagctggcgaccgcaatcgcattcgacgaagcgtcgcggcgaaacgtggacctggtggcgctgcacgcatggagcgacgtcgatgtgtcggagtggcccggaatcgattggccggcaactcagtcgatggccgagcaggtgctggccgagcggttggcgggttggcaggagcggtatcccaacgtagccataacccgcgtggtggtgcgcgatcagccggcccgccagctcgtccaacgctccgaggaagcccagctggtcgtggtcggcagccggggccgcggcggctacgccggaatgctggtggggtcggtaggcgaaaccgttgctcagctggcgcggacgccggtcatcgtggcacgcgagtcgctgacttag

SEQ ID NO: 63 - protein, 96 aa, Mycobacterium tuberculosis
MGLVPRGSHMPYTVRFTTTARRDLHKLPPRILAAVVEFAFGDLSREPLRVGKPLRRELAGTFSARRGTYRLLYRIDDEHTTVVILRVDHRADIYRR

SEQ ID NO: 64 - DNA, 270 bp, Mycobacterium tuberculosis
atgccttacaccgtgcggttcaccacaaccgcgcgtcgagacctccacaagctgccaccgcgcatcctcgcggcagtggtcgaattcgcgttcggcgatctgtcgcgcgagcccctgcgggtgggcaagccccttcggcgcgagttggccggcacgttcagcgcgcgtcgcggaacgtaccgcctgctgtaccggattgacgacgagcacacaacggtagtgatcctgcgcgtcgatcaccgcgcggacatctaccgccgatagaagctt

SEQ ID NO: 65 - protein, 180 aa, Mycobacterium tuberculosis
MINVQAKPAAAASLAAIAIAFLAGCSSTKPVSQDTSPKPATSPAAPVTTAAMADPAADLIGRGCAQYAAQNPTGPGSVAGMAQDPVATAASNNPMLSTLTSALSGKLNPDVNLVDTLNGGEYTVFAPTNAAFDKLPAATIDQLKTDAKLLSSILTYHVIAGQASPSRIDGTHQTLQGADL

SEQ ID NO: 66 - DNA, 663 bp, Mycobacterium tuberculosis
atgatcaacgttcaggccaaaccggccgcagcagcgagcctcgcagccatcgcgattgcgttcttagcgggttgttcgagcaccaaacccgtgtcgcaagacaccagcccgaaaccggcgaccagcccggcggcgcccgttaccacggcggcaatggctgaccccgcagcggacctgattggtcgtgggtgcgcgcaatacgcggcgcaaaatcccaccggtcccggatcggtggccggaatggcgcaagacccggtcgctaccgcggcttccaacaacccgatgctcagtaccctgacctcggctctgtcgggcaagctgaacccggatgtgaatctggtcgacaccctcaacggcggcgagtacaccgttttcgcccccaccaacgccgcattcgacaagctgccggcggccactatcgatcaactcaagactgacgccaagctgctcagcagcatcctgacctaccacgtgatagccggccaggcgagtccgagcaggatcgacggcacccatcagaccctgcaaggtgccgacctgacggtgataggcgcccgcgacgacctcatggtcaacaacgccggtttggtatgtggcggagttcacaccgccaacgcgacggtgtacatgatcgatacggtgctgatgcccccggcacagtaa

SEQ ID NO: 67 - protein, 193 aa, Mycobacterium tuberculosis
MKVKNTIAATSFAAAGLAALAVAVSPPAAAGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 68 - DNA, 582 bp, Mycobacterium tuberculosis
atgaaggtaaagaacacaattgcggcaaccagtttcgcggcggccggcctggcggctctggcggtggctgtctcaccgccggcggccgcaggcgatctggtgggcccgggctgcgcggaatacgcggcagccaatcccactgggccggcctcggtgcagggaatgtcgcaggacccggtcgcggtggcggcctcgaacaatccggagttgacaacgctgacggctgcactgtcgggccagctcaatccgcaagtaaacctggtggacaccctcaacagcggtcagtacacggtgttcgcaccgaccaacgcggcatttagcaagctgccggcatccacgatcgacgagctcaagaccaattcgtcactgctgaccagcatcctgacctaccacgtagtggccggccaaaccagcccggccaacgtcgtcggcacccgtcagaccctccagggcgccagcgtgacggtgaccggtcagggtaacagcctcaaggtcggtaacgccgacgtcgtctgtggtggggtgtctaccgccaacgcgacggtgtacatgattgacagcgtgctaatgcctccggcgtaa

SEQ ID NO: 69 - protein, 97 aa, Mycobacterium tuberculosis
MSLLDAHIPQLIASHTAFAAKAGLMRHTIGQAEQQAMSAQAFHQGESAAAFQGAHARFVAAAAKVNTLLDIAQANLGEAAGTYVAADAAAASSYTGF

SEQ ID NO: 70 - DNA, 294 bp, Mycobacterium tuberculosis
atgagtttgttggatgcccatattccgcagttgatcgcttcgcatacggcgtttgccgctaaggcggggttgatgcggcatacgatcggtcaggccgagcagcaggcgatgtcggcgcaggcgtttcatcagggagagtccgcggcggcgtttcagggtgcgcatgcccggtttgtggccgcggccgccaaggtcaataccttgctggatatcgcgcaagccaatttgggtgaggccgcgggcacgtatgtggccgccgatgccgccgccgcgtccagctacaccgggttttaa

SEQ ID NO: 71 - protein, 334 aa, Mycobacterium tuberculosis
MHKASQSMITPTTQIAGAGVLGNDRKPDESCARAAAAADPGPPTRPAHNAAGVSPEMVQVPAEAQRIVVLSGDQLDALCALGLQSRIVAAALPNSSSSQPSYLGTTVHDLPGVGTRSAPDLRAIAAAHPDLILGSQGLTPQLYPQLAAIAPTVFTAAPGADWENNLRGVGAATARIAAVDALITGFAEHATQVGTKHDATHFQASIVQLTANTMRVYGANNFPASVLSAVGVDRPPSQRFTDKAYIEIGTTAADLAKSPDFSAADADIVYLSCASEAAAERAAVILDSDPWRKLSANRDNRVFVVNDQVWQTGEGMVAARGIVDDLRWVDAPIN

SEQ ID NO: 72 - DNA, 1005 bp, Mycobacterium tuberculosis
atgcataaggcgtcacaatcgatgatcacgcccaccacccagatcgccggcgccggggtgctgggaaacgacagaaagccggatgagtcgtgcgcgcgtgcggcggccgcggccgatccggggccaccgacccgaccagcgcacaatgcggcgggagtcagcccggagatggtgcaggtgccggcggaggcgcagcgcatcgtggtgctctccggtgaccagctcgacgcgctgtgcgcgctgggcctgcaatcgcggatcgtcgccgccgcgttgccgaacagctcctcaagtcaaccttcctatctgggcacgaccgtgcatgatctgcccggtgtcggtactcgcagcgcccccgacctgcgcgccattgcggcggctcacccggatctgatcctgggttcgcagggtttgacgccgcagttgtatccgcagctggcggcgatcgccccgacggtgtttaccgcggcaccgggcgcggactgggaaaataacctgcgtggtgtcggtgccgccacggcccgtatcgccgcggtggacgcgctgatcaccgggttcgccgaacacgccacccaggtcgggaccaagcatgacgcgacccacttccaagcgtcgatcgtgcagctgaccgccaacaccatgcgggtatacggcgccaacaacttcccggccagcgtgctgagcgcggtcggcgtcgaccgaccgccgtctcaacggttcaccgacaaggcctacatcgagatcggcaccacggccgccgacctggcgaaatcaccggacttctcggcggccgacgccgatatcgtctacctgtcgtgcgcgtcggaagcagccgcggaacgcgcggccgtcatcctggatagcgacccatggcgcaagctgtccgccaaccgtgacaaccgggtcttcgtcgtcaacgaccaggtatggcagaccggcgagggtatggtcgctgcccgcggcattgtcgatgatctgcgctgggtcgacgcgccgatcaactag

SEQ ID NO: 73 - protein, 299 aa, Mycobacterium tuberculosis
MLRGIQALSRPLTRVYRALAVIGVLAASLLASWVGAVPQVGLAASALPTFAHVVIVVEENRSQAAIIGNKSAPFINSLAANGAMMAQAFAETHPSEPNYLALFAGNTFGLTKNTCPVNGGALPNLGSELLSAGYTFMGFAEDLPAVGSTVCSAGKYARKHVPWVNFSNVPTTLSVPFSAFPKPQNYPGLPTVSFVIPNADNDMHDGSIAQGDAWLNRHLSAYANWAKTNNSLLVVTWDEDDGSSRNQIPTVFYGAHVRPGTYNETISHYNVLSTLEQIYGLPKTGYATNAPPITDIWGD

SEQ ID NO: 74 - DNA, 900 bp, Mycobacterium tuberculosis
atgctccgcggaatccaggctctcagccggcccctgaccagggtataccgtgccttggcggtgatcggtgtcctggcagcatcgttgctggcctcatgggtcggcgctgtcccacaagtgggtctggcagcgagtgccctgccgaccttcgcgcacgtggtcatcgtggtggaggagaaccgctcgcaggccgccatcatcggtaacaagtcggctcccttcatcaattcgctggccgccaacggcgcgatgatggcccaggcgttcgccgaaacacacccgagcgaaccgaactacctggcactgttcgctggcaacacattcgggttgacgaagaacacctgccccgtcaacggcggcgcgctgcccaacctgggttctgagttgctcagcgccggttacacattcatggggttcgccgaagacttgcctgcggtcggctccacggtgtgcagtgcgggcaaatacgcacgcaaacacgtgccgtgggtcaacttcagtaacgtgccgacgacactgtcggtgccgttttcggcatttccgaagccgcagaattaccccggcctgccgacggtgtcgtttgtcatccctaacgccgacaacgacatgcacgacggctcgatcgcccaaggcgacgcctggctgaaccgccacctgtcggcatatgccaactgggccaagacaaacaacagcctgctcgttgtgacctgggacgaagacgacggcagcagccgcaatcagatcccgacggtgttctacggcgcgcacgtgcggcccggaacttacaacgagaccatcagccactacaacgtgctgtccacattggagcagatctacggactgcccaagacgggttatgcgaccaatgctccgccaataaccgatatttggggcgactag

SEQ ID NO: 75 - protein, 99 aa, Mycobacterium tuberculosis
MRATVGLVEAIGIRELRQHASRYLARVEAGEELGVTNKGRLVARLIPVQAAERSREALIESGVLIPARRPQNLLDVTAEPARGRKRTLSDVLNEMRDEQ

SEQ ID NO: 76 - DNA, 300 bp, Mycobacterium tuberculosis
atgcgtgctaccgttgggcttgtggaggcaatcggaatccgagaactaagacagcacgcatcgcgatacctcgcccgggttgaagccggcgaggaacttggcgtcaccaacaaaggaagacttgtggcccgactcatcccggtgcaggccgcggagcgttctcgcgaagccctgattgaatcaggtgtcctgattccggctcgtcgtccacaaaaccttctcgacgtcaccgccgaaccggcgcgcggccgcaagcgcaccctgtccgatgttctcaacgaaatgcgcgacgagcagtga

SEQ ID NO: 77 - protein, 217 aa, Mycobacterium tuberculosis
VAIANPAEPGAAGRHHQPRGDRKPRAWRQCGPQNGPRRSQAITPEPGAAGRHHQPRGDRKPRAWRQCGPQNGPRRSQAITPEPGAAGRHHQPRGDRKPRAWRQCGPQNGPRRSQAITPEPGAAGRHHQPRGDRKPRAWRQCGPQNGPRRSQAITPEPGAAGRHHQPRGDRKPRAWRQCGPQNGPRRSQAITPEPGAAGRHWLDQRPVVPDGVGKSDS

SEQ ID NO: 78 - DNA, 654 bp, Mycobacterium tuberculosis
gtggcgatcgcaaacccggcggagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccactggctagaccaacgaccggtagttcccgacggcgtcggaaaatccgacagctga

SEQ ID NO: 79 - protein, 185 aa, Mycobacterium tuberculosis
HMDLPGNDFDSNDFDAVDLWGADGAEGWTADPIIGVGSAATPDTGPDLDNAHGQAETDTEQEIALFTVTNPPRTVSVSTLMDGRIDHVELSARVAWMSESQLASEILVIADLARQKAQSAQYAFILDRMSQQVDADEHRVALLRKTVGETWGLPSPEEAAAAEAEVFATRYSDDCPAPDDESDPW

SEQ ID NO: 80 - DNA, 555 bp, Mycobacterium tuberculosis
gtggacttgcccggaaatgactttgacagcaacgatttcgacgccgtggatctctggggtgccgacggcgcggagggctggactgcggatccgattattggcgtcgggtcggcggcgaccccggacaccggacccgacctggacaatgcccacggtcaggcggagacggacaccgaacaagagatcgcgctttttaccgtgacgaatcccccacgcacggtgtcggtatcgacgctgatggacggccggattgaccatgtcgagctgtcggccagggtggcctggatgagtgagtcgcagctcgcttctgagatcctggtgattgccgacctggcgcggcagaaggcgcagtcggcccagtacgccttcatccttgacaggatgagtcaacaggtcgatgcagatgaacaccgcgtcgcactgctacgtaagaccgtgggcgaaacctgggggttaccatcgccggaagaagccgcggcagcagaagctgaggtgttcgcgacgcgctacagcgacgattgtccagcaccagacgacgagagcgatccatggtga

SEQ ID NO: 81 - protein, 392 aa, Mycobacterium tuberculosis
MSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVV

SEQ ID NO: 82 - DNA, 1179 bp, Mycobacterium tuberculosis
atgagcagagcgttcatcatcgatccaacgatcagtgccattgacggcttgtacgaccttctggggattggaatacccaaccaagggggtatcctttactcctcactagagtacttcgaaaaagccctggaggagctggcagcagcgtttccgggtgatggctggttaggttcggccgcggacaaatacgccggcaaaaaccgcaaccacgtgaattttttccaggaactggcagacctcgatcgtcagctcatcagcctgatccacgaccaggccaacgcggtccagacgacccgcgacatcctggagggcgccaagaaaggtctcgagttcgtgcgcccggtggctgtggacctgacctacatcccggtcgtcgggcacgccctatcggccgccttccaggcgccgttttgcgcgggcgcgatggccgtagtgggcggcgcgcttgcctacttggtcgtgaaaacgctgatcaacgcgactcaactcctcaaattgcttgccaaattggcggagttggtcgcggccgccattgcggacatcatttcggatgtggcggacatcatcaagggcaccctcggagaagtgtgggagttcatcacaaacgcgctcaacggcctgaaagagctttgggacaagctcacggggtgggtgaccggactgttctctcgagggtggtcgaacctggagtccttctttgcgggcgtccccggcttgaccggcgcgaccagcggcttgtcgcaagtgactggcttgttcggtgcggccggtctgtccgcatcgtcgggcttggctcacgcggatagcctggcgagctcagccagcttgcccgccctggccggcattgggggcgggtccggttttgggggcttgccgagcctggctcaggtccatgccgcctcaactcggcaggcgctacggccccgagctgatggcccggtcggcgccgctgccgagcaggtcggcgggcagtcgcagctggtctccgcgcagggttcccaaggtatgggcggacccgtaggcatgggcggcatgcacccctcttcgggggcgtcgaaagggacgacgacgaagaagtactcggaaggcgcggcggcgggcactgaagacgccgagcgcgcgccagtcgaagctgacgcgggcggtgggcaaaaggtgctggtacgaaacgtcgtctaa

SEQ ID NO: 83 - protein, 94 aa, Mycobacterium tuberculosis
MTINYQFGDVDAHGAMIRAQAGSLEAEHQAIISDVLTASDFWGGAGSAACQGFITQLGRNFQVIYEQANAHGQKVQAAGNNMAQTDSAVGSSWA

SEQ ID NO: 84 - DNA, 285 bp, Mycobacterium tuberculosis
atgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggcctaa

SEQ ID NO: 85 - protein, 162 aa, Mycobacterium tuberculosis
VQFDVTIEIPKGQRNKYEVDHETGRVRLDRYLYTPMAYPTDYGFIEDTLGDDGDPLDALVLLPQPVFPGVLVAARPVGMFRMVDEHGGDDKVLCVPAGDPRWDHVQDIGDVPAFELDAIKHFFVHYKDLEPGKFVKAADWVDRAEAEAEVQRSVERFKAGTH

SEQ ID NO: 86 - DNA, 489 bp, Mycobacterium tuberculosis
gtgcaattcgacgtgaccatcgaaattcccaagggccagcgcaacaaatacgaggtcgaccatgagacggggcgggttcgtctggaccggtacctgtacaccccgatggcctacccgaccgactacggcttcatcgaggacaccctaggtgacgatggcgacccgctggacgcgctggtgctgctaccgcagccggtcttccccggggtgctggtggcggcgcggccggtggggatgttccggatggtcgacgagcacggcggcgacgacaaagtgctgtgcgtcccagccggtgacccccggtgggaccacgtccaagacatcggggacgttccggctttcgagctggatgcgatcaagcatttctttgtgcactacaaggacctggaaccaggtaagttcgtcaaggcggccgactgggtcgaccgcgccgaagccgaggcagaggtgcagcgttcagtggagcgcttcaaggccggtacacactga

SEQ ID NO: 87 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 88 - DNA, 1018 bp, Mycobacterium tuberculosis
atgcagcttgttgacagggttcgtggcgccgtcacgggtatgtcgcgtcgactcgtggtcggggccgtcggcgcggccctagtgtcgggtctggtcggcgccgtcggtggcacggcgaccgcgggggcattttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggcgcgcagctcaacgctatgaagcccgacctgcaacgggcactgggtgccacgcccaacaccgggcccgcgccccagggcgcctaga

SEQ ID NO: 89 - protein, 181 aa, Mycobacterium tuberculosis
MTEYEGPKTKFHALMQEQIHNEFTAAQQYVAIAVYFDSEDLPQLAKHFYSQAVEERNHAMMLVQHLLDRDLRVEIPGVDTVRNQFDRPREALALALDQERTVTDQVGRLTAVARDEGDFLGEQFMQWFLQEQIEEVALMATLVRVADRAGANLFELENFVAREVDVAPAASGAPHAAGGRL

SEQ ID NO: 90 - DNA, 546 bp, Mycobacterium tuberculosis
atgacagaatacgaagggcctaagacaaaattccacgcgttaatgcaggaacagattcataacgaattcacagcggcacaacaatatgtcgcgatcgcggtttatttcgacagcgaagacctgccgcagttggcgaagcatttttacagccaagcggtcgaggaacgaaaccatgcaatgatgctcgtgcaacacctgctcgaccgcgaccttcgtgtcgaaattcccggcgtagacacggtgcgaaaccagttcgacagaccccgcgaggcactggcgctggcgctcgatcaggaacgcacagtcaccgaccaggtcggtcggctgacagcggtggcccgcgacgagggcgatttcctcggcgagcagttcatgcagtggttcttgcaggaacagatcgaagaggtggccttgatggcaaccctggtgcgggttgccgatcgggccggggccaacctgttcgagctagagaacttcgtcgcacgtgaagtggatgtggcgccggccgcatcaggcgccccgcacgctgccgggggccgcctctag

SEQ ID NO: 91 - protein, 402 aa, Mycobacterium tuberculosis
MASGSGLCKTTSNFIWGQLLLLGEGIPDPGDIFNTGSSLFKQISDKMGLAIPGTNWIGQAAEAYLNQNIAQQLRAQVMGDLDKLTGNMISNQAKYVSDTRDVLRAMKKMIDGVYKVCKGLEKIPLLGHLWSWELAIPMSGIAMAVVGGALLYLTIMTLMNATNLRGILGRLIEMLTTLPKFPGLPGLPSLPDIIDGLWPPKLPDIPIPGLPDIPGLPDFKWPPTPGSPLFPDLPSFPGFPGFPEFPAIPGFPALPGLPSIPNLFPGLPGLGDLLPGVGDLGKLPTWTELAALPDFLGGFAGLPSLGFGNLLSFASLPTVGQVTATMGQLQQLVAAGGGPSQLASMGSQQAQLISSQAQQGGQQHATLVSDKKEDEEGVAEAERAPIDAGTAASQRGQEGTVL

SEQ ID NO: 92 - DNA, 1209 bp, Mycobacterium tuberculosis
atggcatcgggtagcggtctttgcaagacgacgagtaactttatttggggccagttactcttgcttggagagggaatccccgacccaggcgacattttcaacaccggttcgtcgctgttcaaacaaatcagcgacaaaatgggactcgccattccgggcaccaactggatcggccaagcggcggaagcttacctaaaccagaacatcgcgcaacaacttcgcgcacaggtgatgggcgatctcgacaaattaaccggcaacatgatctcgaatcaggccaaatacgtctccgatacgcgcgacgtcctgcgggccatgaagaagatgattgacggtgtctacaaggtttgtaagggcctcgaaaagattccgctgctcggccacttgtggtcgtgggagctcgcaatccctatgtccggcatcgcgatggccgttgtcggcggcgcattgctctatctaacgattatgacgctgatgaatgcgaccaacctgaggggaattctcggcaggctgatcgagatgttgacgaccttgccaaagttccccggcctgcccgggttgcccagcctgcccgacatcatcgacggcctctggccgccgaagttgcccgacattccgatccccggcctgcccgacatcccgggcctacccgacttcaaatggccgcccacccccggcagcccgttgttccccgacctcccgtcgttcccagggttccccgggttcccggagttccccgccatccccgggttccccgcactgcccgggttgcccagcattcccaacttgttccccggcttgccgggtctgggcgacctgctgcccggcgtaggcgatttgggcaagttacccacctggactgagctggccgctttgcctgacttcttgggcggcttcgccggcctgcccagcttgggttttggcaatctgctcagctttgccagtttgcccaccgtgggtcaggtgaccgccaccatgggtcagctgcaacagctcgtggcggccggcggtggccccagccaactggccagcatgggcagccaacaagcgcaactgatctcgtcgcaggcccagcaaggaggccagcagcacgccaccctcgtgagcgacaagaaggaagacgaggaaggcgtggccgaggcggagcgtgcacccatcgacgctggcaccgcggccagccaacgggggcaggaggggaccgtcctttga

SEQ ID NO: 93 - protein, 100 aa, Mycobacterium tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 94 - DNA, 303 bp, Mycobacterium tuberculosis
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttctga

SEQ ID NO: 95 - protein, 461 aa, Mycobacterium tuberculosis
HMTQSQTVTVDQQEILNRANEVEAPMADPPTDVPITPCELTAAKNAAQQLVLSADNMREYLAAGAKERQRLATSLRNAAKAYGEVDEEAATALDNDGEGTVQAESAGAVGGDSSAELTDTPRVATAGEPNFMDLKEAARKLETGDQGASLAHFADGWNTFNLTLQGDVKRFRGFDNWEGDAATACEASLDQQRQWILHMAKLSAAMAKQAQYVAQLHVWARREHPTYEDIVGLERLYAENPSARDQILPVYAEYQQRSEKVLTEYNNKAALEPVNPPKPPPAIKIDPPPPPQEQGLIPGFLMPPSDGSGVTPGTGMPAAPMVPPTGSPGGGLPADTAAQLTSAGREAAALSGDVAVKAASLGGGGGGGVPSAPLGSAIGGAESVRPAGAGDIAGLGQGRAGGGAALGGGGMGMPMGAAHQGQGGAKSKGSQQEDEALYTEDRAWTEAVIGNRRRQDSKESK

SEQ ID NO: 96 - DNA, 1383 bp, Mycobacterium tuberculosis
atgacgcagtcgcagaccgtgacggtggatcagcaagagattttgaacagggccaacgaggtggaggccccgatggcggacccaccgactgatgtccccatcacaccgtgcgaactcacggcggctaaaaacgccgcccaacagctggtattgtccgccgacaacatgcgggaatacctggcggccggtgccaaagagcggcagcgtctggcgacctcgctgcgcaacgcggccaaggcgtatggcgaggttgatgaggaggctgcgaccgcgctggacaacgacggcgaaggaactgtgcaggcagaatcggccggggccgtcggaggggacagttcggccgaactaaccgatacgccgagggtggccacggccggtgaacccaacttcatggatctcaaagaagcggcaaggaagctcgaaacgggcgaccaaggcgcatcgctcgcgcactttgcggatgggtggaacactttcaacctgacgctgcaaggcgacgtcaagcggttccgggggtttgacaactgggaaggcgatgcggctaccgcttgcgaggcttcgctcgatcaacaacggcaatggatactccacatggccaaattgagcgctgcgatggccaagcaggctcaatatgtcgcgcagctgcacgtgtgggctaggcgggaacatccgacttatgaagacatagtcgggctcgaacggctttacgcggaaaacccttcggcccgcgaccaaattctcccggtgtacgcggagtatcagcagaggtcggagaaggtgctgaccgaatacaacaacaaggcagccctggaaccggtaaacccgccgaagcctccccccgccatcaagatcgacccgcccccgcctccgcaagagcagggattgatccctggcttcctgatgccgccgtctgacggctccggtgtgactcccggtaccgggatgccagccgcaccgatggttccgcctaccggatcgccgggtggtggcctcccggctgacacggcggcacagctgacgtcggctgggcgggaagccgcagcgctgtcgggcgacgtggcggtcaaagcggcatcgctcggtggcggtggaggcggcggggtgccgtcggcgccgttgggatccgcgatcgggggcgccgaatcggtgcggcccgctggcgctggtgacattgccggcttaggccagggaagggccggcggcggcgccgcgctgggcggcggtggcatgggaatgccgatgggtgccgcgcatcagggacaagggggcgccaagtccaagggttctcagcaggaagacgaggcgctctacaccgaggatcgggcatggaccgaggccgtcattggtaaccgtcggcgccaggacagtaaggagtcgaagtga

SEQ ID NO: 97 - protein, 838 aa, Artificial Sequence
  fusion polypeptide referred to as DID90A, which contains sequences from Rv2031, Rv0934 and Rv2032
HMHHHHHHMATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTNKLCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISSELMPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHR

SEQ ID NO: 98 - protein, 857 aa, Artificial Sequence
  fusion polypeptide referred to as DID90B, which contains sequences from Rv2875, Rv0934 and Rv2032
HMHHHHHHGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPAKLCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISSELMPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHR

SEQ ID NO: 99 - protein, 965 aa, Artificial Sequence
  fusion polypeptide referred to as DID104, which contains sequences from Rv0831, Rv0934 and Rv2032
HMHHHHHHMLPETNQDEVQPNAPVALVTVEIRHPTTDSLTESANRELKHLLINDLPIERQAQDVSWGMTAPGGAPTPVADRFVRYVNRDNTTAASLKNQAIVVETTAYRSFEAFTDVVMRVVDARAQVSSIVGLERIGLRFVLEIRVPAGVDGRITWSNWIDEQLLGPQRFTPGGLVLTEWQGAAVYRELQPGKSLIVRYGPGMGQALDPNYHLRRITPAQTGPFFLLDIDSFWTPSGGSIPEYNRDALVSTFQDLYGPAQVVFQEMITSRLKDELLRQKLCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISSELMPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHR

SEQ ID NO: 100 - protein, 607 aa, Artificial Sequence
  fusion polypeptide referred to as DID64, which contains sequences from Rv2031, Rv0934 and Rv3874
HMHHHHHHMATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTNKLCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISSELMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 101 - protein, 623 aa, Artificial Sequence
  fusion polypeptide referred to as DID65, which contains sequences from Rv2875, Rv0934 and Rv3874
HMHHHHHHGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPAKLCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISSELMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 102 - protein, 790 aa, Artificial Sequence
  fusion polypeptide referred to as DID8265, which contains sequences from Rv2875, Rv1860 and Rv2032
HMHHHHHHGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPADPEPAPPVPTTAASPPSTAAAPPAPATPVAPPPPAAANTPNAQPGDPNAAPPPADPNAPPPPVIAPNAPQPVRIDNPVGGFSFALPAGWVESDAAHFDYGSALLSKTTGDPPFPGQPPPVANDTRIVLGRLDQKLYASAEATDSKAAARLGSDMGEFYMPYPGTRINQETVSLDANGVSGSASYYEVKFSDPSKPNGQIWTGVIGSPAANAPDAGPPQRWFVVWLGTANNPVDKGAAKALAESIRPLVAPPPAPAPAPAEPAPAPAPAGEVAPTPTTPTPQRTLPAELMPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHR

SEQ ID NO: 103 - protein, 910 aa, Artificial Sequence
  fusion polypeptide referred to as DID96, which contains sequences from Rv0632, Rv1980 and Rv3881
HMHHHHHHMSDPVSYTRKDSIAVISMDDGKVNALGPAMQQALNAAIDNADRDDVGALVITGNGRVFSGGFDLKILTSGEVQPAIDMLRGGFELAYRLLSYPKPVVMACTGHAIAMGAFLLSCGDHRVAAHAYNIQANEVAIGMTIPYAALEIMKLRLTRSAYQQATGLAKTFFGETALAAGFIDEIALPEVVVSRAEEAAREFAGLNQHAHAATKLRSRADALTAIRAGIDGIAAEFGLELMAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAELHMTQSQTVTVDQQEILNRANEVEAPMADPPTDVPITPCELTAAKNAAQQLVLSADNMREYLAAGAKERQRLATSLRNAAKAYGEVDEEAATALDNDGEGTVQAESAGAVGGDSSAELTDTPRVATAGEPNFMDLKEAARKLETGDQGASLAHFADGWNTFNLTLQGDVKRFRGFDNWEGDAATACEASLDQQRQWILHMAKLSAAMAKQAQYVAQLHVWARREHPTYEDIVGLERLYAENPSARDQILPVYAEYQQRSEKVLTEYNNKAALEPVNPPKPPPAIKIDPPPPPQEQGLIPGFLMPPSDGSGVTPGTGMPAAPMVPPTGSPGGGLPADTAAQLTSAGREAAALSGDVAVKAASLGGGGGGGVPSAPLGSAIGGAESVRPAGAGDIAGLGQGRAGGGAALGGGGMGMPMGAAHQGQGGAKSKGSQQEDEALYTEDRAWTEAVIGNRRRQDSKESK

SEQ ID NO: 104 - protein, 905 aa, Artificial Sequence
  fusion polypeptide referred to as DID94, which contains sequences from Rv1860, Rv1980 and Rv3864
MHHHHHHMDPEPAPPVPTTAASPPSTAAAPPAPATPVAPPPPAAANTPNAQPGDPNAAPPPADPNAPPPPVIAPNAPQPVRIDNPVGGFSFALPAGWVESDAAHFDYGSALLSKTTGDPPFPGQPPPVANDTRIVLGRLDQKLYASAEATDSKAAARLGSDMGEFYMPYPGTRINQETVSLDANGVSGSASYYEVKFSDPSKPNGQIWTGVIGSPAANAPDAGPPQRWFVVWLGTANNPVDKGAAKALAESIRPLVAPPPAPAPAPAEPAPAPAPAGEVAPTPTTPTPQRTLPAELAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAGSMASGSGLCKTTSNFIWGQLLLLGEGIPDPGDIFNTGSSLFKQISDKMGLAIPGTNWIGQAAEAYLNQNIAQQLRAQVMGDLDKLTGNMISNQAKYVSDTRDVLRAMKKMIDGVYKVCKGLEKIPLLGHLWSWELAIPMSGIAMAVVGGALLYLTIMTLMNATNLRGILGRLIEMLTTLPKFPGLPGLPSLPDIIDGLWPPKLPDIPIPGLPDIPGLPDFKWPPTPGSPLFPDLPSFPGFPGFPEFPAIPGFPALPGLPSIPNLFPGLPGLGDLLPGVGDLGKLPTWTELAALPDFLGGFAGLPSLGFGNLLSFASLPTVGQVTATMGQLQQLVAAGGGPSQLASMGSQQAQLISSQAQQGGQQHATLVSDKKEDEEGVAEAERAPIDAGTAASQRGQEGTVL